(19) United States
(12) Patent Application Publication
(10) Pub. No.: US 2026/0108545 A1
(43) Pub. Date: Apr. 23, 2026

(54) Methods of treating fibromyalgia with compositions comprising psilocybin

(71) Applicant: Tryp Therapeutics Inc., Kelowna (CA)

(72) Inventors: James GILLIGAN, Basking Ridge, NJ (US); Peter GUZZO, Niskayuna, NY (US)

(73) Assignee: Tryp Therapeutics Inc., Kelowna, BC (CA)

(21) Appl. No.: 19/110,772

(22) PCT Filed: Sep. 11, 2023
(86) PCT No.: PCT/IB2023/059010

§ 371 Date: Mar. 11, 2025

Related U.S. Application Data
(60) Provisional application No. 63/405,786, filed on Sep. 12, 2022.

(51) Int. Cl. A61K 31/675; A61B 5/00; A61B 5/0245; A61B 5/055; A61B 5/369; A61K 9/00; A61P 25/04; G01R 33/48

(52) U.S. Cl. CPC A61K 31/675; A61B 5/0245; A61B 5/055; A61B 5/369; A61B 5/4809; A61B 5/4815; A61B 5/4827; A61B 5/4848; A61K 9/0053; A61P 25/04; G01R 33/4806

(57) ABSTRACT

Provided are methods for treating fibromyalgia or one or more symptoms thereof, that involve the administration of psychedelics, such as psilocybin or a metabolite thereof, and related uses of the psychedelics. The disclosure in some aspects also relates to methods for identifying subjects for treatment of fibromyalgia with administration of psychedelics. In some aspects, the treatment also involves psychotherapy. In some aspects, the subject receiving the treatment is assessed for various indicators, such as observer-rated and subject-reported outcomes, biological and clinical indicators, and combinations thereof.

### CROSS-REFERENCE TO RELATED APPLICATIONS

This application claims priority from U.S. Provisional Application No. 63/405,786, filed Sep. 12, 2022, entitled “COMPOSITIONS AND METHODS FOR TREATING FIBROMYALGIA,” the contents of which are incorporated by reference in their entirety.

### FIELD

The present disclosure relates in some aspects to methods for treating fibromyalgia or one or more symptoms thereof, that involve the administration of psychedelics, such as psilocybin or a metabolite thereof, and related uses of the psychedelics. The disclosure in some aspects also relates to methods for identifying subjects for treatment of fibromyalgia with administration of psychedelics. In some aspects, the treatment also involves psychotherapy. In some aspects, the subject receiving the treatment is assessed for various indicators, such as observer-rated and subject-reported outcomes, biological and clinical indicators, and combinations thereof.

### BACKGROUND

Fibromyalgia is a chronic syndrome of widespread musculoskeletal pain that can include manifestation of multiple co-occurring symptoms, including sleep disturbances, fatigue, cognitive dysfunction, and mood problems including anxiety and depression. While treatments and other approaches are available for managing fibromyalgia, existing treatments only provide a modest benefit and are accompanied by substantial side effect burden. Improved therapeutic approaches are needed. Provided are embodiments that meet such needs.

### SUMMARY

Provided herein are methods of treating fibromyalgia (FM) in a subject. In some of the provided embodiments, the methods involve administering to a subject suffering from one or more symptoms of FM, one or more doses of psilocybin or an active metabolite thereof effective to induce a dissociative state. Also provided are related uses, such as therapeutic uses or uses in the manufacture of a medicament, of one or more doses of psilocybin or an active metabolite thereof, or a composition comprising psilocybin or an active metabolite, for example, in accordance with any of the provided embodiments.

Provided herein are methods of treating FM that involve orally administering to a subject suffering from one or more symptoms of FM, one or more doses of psilocybin or an active metabolite thereof effective to induce a dissociative state.

In some of any embodiments, the methods also involve providing one or more integration sessions to the subject after emergence from the dissociative state.

In some of any embodiments, the methods also involve assessing in the subject one or more indicators associated with the one or more symptoms of FM, dissociative state, treatment outcome, safety and/or is related to the subject, prior to and/or after administration of the one or more doses of psilocybin.

Provided herein are methods of treating FM that involve administering to a subject suffering from one or more symptoms of FM, one or more doses of psilocybin or an active metabolite thereof effective to induce a dissociative state; providing one or more integration sessions to the subject after emergence from the dissociative state; and assessing in the subject one or more indicators associated with the one or more symptoms of FM, dissociative state, treatment outcome, safety and/or is related to the subject, prior to and/or after administration of the one or more doses of psilocybin or an active metabolite thereof.

Provided herein are methods of treating FM that involve assessing in a subject suffering from one or more symptoms of FM, one or more indicators associated with the one or more symptoms of FM, dissociative state, treatment outcome, safety and/or is related to the subject, prior to and/or after administration of one or more doses of psilocybin or an active metabolite thereof effective to induce a dissociative state and one or more integration sessions after emergence from a dissociative state induced by the one or more doses of psilocybin or an active metabolite thereof.

Provided herein are methods of treating FM that involve administering to a subject suffering from one or more symptoms of FM, one or more doses of psilocybin or an active metabolite thereof effective to induce a dissociative state; providing one or more integration sessions to the subject after emergence from the dissociative state; and assessing in the subject one or more indicators prior to and/or after administration of the one or more doses of psilocybin or an active metabolite thereof; wherein the one or more indicators comprises one or more of: a fibromyalgia survey score, a pain interference assessment, a sleep assessment, a chronic pain acceptance assessment, or a clinical indicator assessment.

In some of any embodiments, the methods also involve, prior to administering the one or more doses of psilocybin, assessing one or more of the following exclusion parameters in the subject: (1) use of prior or concomitant medications; (2) hypertension and/or tachycardia; (3) incidence of epilepsy; (4) incidence of schizophrenia spectrum or other psychotic disorders; (5) a moderate or severe alcohol or drug use disorder; and (6) prior adverse effects from psilocybin.

In some of any embodiments, the methods also involve identifying the subject for treatment with psilocybin if the subject does not meet any of the exclusion parameters.

Provided herein are methods of identifying a subject for treating FM with psilocybin or an active metabolite thereof that involve (a) assessing in a subject suffering from one or more symptoms of FM, one or more of the following exclusion parameters: (1) use of prior or concomitant medications; (2) hypertension and/or tachycardia; (3) incidence of epilepsy; (4) incidence of schizophrenia spectrum or other psychotic disorders; (5) a moderate or severe alcohol or drug use disorder; and (6) prior adverse effects from psilocybin or an active metabolite thereof. (b) identifying the subject for treatment with one or more doses of psilocybin or an active metabolite thereof if the subject does not meet any of the exclusion parameters.

Provided herein are methods of identifying a subject for treating FM with one or more doses of psilocybin or an active metabolite thereof that involve identifying a subject suffering from one or more symptoms of FM for treatment if the subject does not meet any of the following exclusion parameters that are assessed in the subject: (1) use of prior or concomitant medications; (2) hypertension and/or tachycardia; (3) incidence of epilepsy; (4) incidence of schizophrenia spectrum or other psychotic disorders; (5) a moderate or severe alcohol or drug use disorder; and (6) prior adverse effects from psilocybin or an active metabolite thereof.

In some of any embodiments, the methods also involve administering to the identified subject, one or more doses of psilocybin or an active metabolite thereof effective to induce a dissociative state; and providing one or more integration sessions to the subject after emergence from the dissociative state.

In some of any embodiments, the methods also involve assessing in the subject one or more indicators associated with the one or more symptoms of FM, dissociative state, treatment outcome, safety and/or is related to the subject, prior to and/or after administration of the one or more doses of psilocybin or an active metabolite thereof.

In some of any embodiments, the administering is by oral administration or intravenous administration. In some of any embodiments, the administering is by oral administration.

In some of any embodiments, the psilocybin or an active metabolite thereof is psilocybin. In some of any embodiments, the psilocybin or an active metabolite thereof is psilocin.

In some of any embodiments, the one or more indicators comprise observer-rated parameters, subject-reported parameters and/or clinical indicators. In some of any embodiments, the one or more indicators are assessed prior to the administration as a baseline measurement, and after the administration as an outcome measurement, and the changes between the baseline measurement and the outcome measurement are determined. In some of any embodiments, the one or more indicators comprises one or more of: a fibromyalgia survey score, a pain interference assessment, a sleep assessment, a chronic pain acceptance assessment, or a clinical indicator assessment.

In some of any embodiments, the one or more indicators comprises one or more of: a fibromyalgia survey score, optionally a 2016 fibromyalgia survey questionnaire (FSQ) assessment; a pain intensity numerical rating scale (PI-NRS) assessment, optionally an 11-item PI-NRS scale; a PROMIS pain interference assessment; a PainDETECT Questionnaire (PD-Q) assessment; a chronic pain acceptance assessment, optionally measured by chronic pain acceptance questionnaire (CPAQ-8) assessment; a pain interference assessment, optionally measured by 4-item PROMIS pain interference scale; and a Complex Medical Symptom Inventory (CMSI). In some of any embodiments, the one or more indicators comprises a fibromyalgia survey score. In some of any embodiments, the fibromyalgia survey score comprises a 2016 fibromyalgia survey questionnaire (FSQ) assessment.

In some of any embodiments, the one or more indicators comprises a pain intensity numeric rating scale (PI-NRS) 11-item pain scale.

In some of any embodiments, the one or more indicators comprises one or more of: a sleep impairment assessment, optionally measured by a PROMIS sleep-related impairment 4a assessment; a PROMIS sleep disturbance 8b assessment; and a STOP-Bang questionnaire. In some of any embodiments, the one or more indicators comprises one or more of: a PROMIS-29+2 Profile, optionally a v2.1 multi-domain assessment; a positive and negative affect score (PANAS) assessment; a patient global impression of change (PGI-C) scale assessment; and a patient acceptable symptom state (PASS) questionnaire. In some of any embodiments, the one or more indicators comprises a PROMIS-29+2 Profile v2.1 multi-domain assessment.

In some of any embodiments, the one or more indicators comprises one or more of: a Patient Health Questionnaire depression scale-8 (PHQ-8); a Columbia Suicide Severity Rating Scale (C-SSRS); a Clinician Global Impression—Improvement (CGI-I) scale; an Emotional Breakthrough Inventory (EBI); a Acceptance and Action Questionnaire-II (AAQ-II); a Patient Global Impression-Improvement (PGI-I); a Hospital Anxiety and Depression Scale (HADS); and a Life Events Checklist (LEC). In some of any embodiments, the one or more indicators comprises a Patient Health Questionnaire depression scale-8 (PHQ-8).

In some of any embodiments, the one or more indicators comprises one or more of: subject-reported mystical experiences assessment, optionally measured by a Mystical Experience Questionnaire (MEQ30); a subject-reported challenging experiences assessment, optionally measured by a Challenging Experience Questionnaire (CEQ27); a subject-reported psychological insight assessment, optionally measured by the Psychological Insight Questionnaire (PIQ); and a subject-reported qualitative written assessment. In some of any embodiments, the one or more indicators comprises therapist-reported monitor rating scale, optionally measured using a Monitor Rating Scale (MRS) form.

In some of any embodiments, the one or more indicators comprises a Quantitative Sensory Testing (QST). In some of any embodiments, a QST is measured using Cuff Pressure Pain assessment, optionally cuff-PPT, cuff-Pain40, cuff-Pain50, and/or cuff-tolerance.

In some of any embodiments, the one or more indicators comprises a Visual Hypersensitivity Assessment. In some of any embodiments, the one or more indicators comprises an electroencephalogram (EEG).

In some of any embodiments, the one or more indicators comprises QST and EEG, and the QST and the EEG are performed concurrently.

In some of any embodiments, the one or more indicators comprises functional magnetic resonance imaging (fMRI). In some of any embodiments, the fMRI comprises one or more of: structural MRI; resting fMRI; proton magnetic resonance spectroscopy (1H-MRS) in the right anterior insula; fMRI with tonic cuff pressure evoked experimental pain; fMRI with block design cuff pressure pain; and diffusion tensor imaging (DTI).

In some of any embodiments, the one or more indicators comprises one or more of: wrist actigraphy, optionally wherein wrist actigraphy is used to measure sleep onset latency; wake after sleep onset; total sleep time; and sleep efficiency (%).

In some of any embodiments, the one or more indicators comprises an electrocardiograms (ECG).

In some of any embodiments, the one or more indicators are measured during one or more of the integration sessions.

In some of any embodiments, the exclusion parameter is the use of prior or concomitant medications, and the subject is identified for treatment if the subject is not using any of the following prior or concomitant medications: regular use of psychoactive prescription medication, opioids, tramadol, or benzodiazepines; concomitant use of antidepressants; regular use of medications having a primary centrally-acting serotonergic effect or monoamine oxidase inhibitors (MAOIs); use of hallucinogens in the past 6 months; concomitant use of sildenafil or tadalafil; concomitant use of alcohol with greater than 0.02% blood alcohol content; antihypertensive medications; UGT1A9 inhibitors, 1A10 inhibitors, regorafenib, rimampicin, phenytoin, eltrombopag, mefenamic acid, diflunisal, niflumic acid, sorafenib, isavuconazole, deferasiroxor, ginseng; aldehyde dehydrogenase inhibitors, alcohol dehydrogenase inhibitors, or disulfiram; amphetamines, buprenorphine, benzodiazepines, cocaine, methamphetamines, Ecstasy (MDMA), morphine, methadone, oxycodone, marijuana, ethyl glucuronide, fentanyl, tramadol, synthetic cannabinoids (K2) regular use of psychoactive prescription medication, opioids, tramadol, or benzodiazepines; concomitant use of antidepressants; regular use of medications having a primary centrally-acting serotonergic effect, monoamine oxidase inhibitors (MAOIs), or selective serotonin reuptake inhibitors (SSRIs), or use of serotonin-acting dietary supplements, 5-hydroxy-tryptophan, or St. John's wort,

In some of any embodiments, the exclusion parameter is hypertension, and the subject is identified for treatment if the subject does not exhibit a systolic blood pressure of greater than at or about 139 mm Hg, a diastolic blood pressure of greater than at or about 89 mm Hg; and/or wherein the exclusion parameter is tachycardia and the subject is identified for treatment if the subject does not exhibit a heart rate of greater than at or about 90 beats per minute (bpm); optionally when measured as an average of three readings.

In some of any embodiments, the exclusion parameter is incidence of epilepsy and the subject is identified for treatment if the subject does not have epilepsy. In some of any embodiments, the exclusion parameter is incidence of psychotic disorders and the subject is identified for treatment if the subject does not have a schizophrenia spectrum or other psychotic disorder that meets the DSM-5 criteria, major depressive disorder with psychotic features, or Bipolar I or Bipolar II Disorder. In some of any embodiments, the exclusion parameter is a moderate or severe alcohol or drug use disorder, and the subject is identified for treatment if the subject does not have a moderate or severe alcohol or drug use disorder that meets the DSM-5 criteria.

In some of any embodiments, the exclusion parameter is prior adverse effects from psilocybin or an active metabolite thereof, and the subject is identified for treatment if the subject has not had a prior adverse effects from psilocybin or an active metabolite thereof.

In some of any embodiments, the subject exhibits amelioration of the one or more indicators between the baseline measurement and the outcome measurement.

In some of any embodiments, the subject receiving treatment shows improvement in one or more symptoms of FM or one or more symptoms of FM are ameliorated. In some of any embodiments, the subject receiving treatment shows improvements selected from among one or more of: a fibromyalgia survey score, a pain interference assessment, a sleep assessment, a chronic pain acceptance assessment, or a clinical indicator assessment. In some of any embodiments, the subject receiving treatment shows improvements selected from among one or more of: a fibromyalgia survey score, optionally a 2016 fibromyalgia survey questionnaire (FSQ) assessment; a pain intensity numerical rating scale (PI-NRS) assessment, optionally an 11-item PI-NRS scale; a PROMIS pain interference assessment; a PainDETECT Questionnaire (PD-Q) assessment; a chronic pain acceptance assessment, optionally measured by chronic pain acceptance questionnaire (CPAQ-8) assessment; and a pain interference assessment, optionally measured by 4-item PROMIS pain interference scale.

In some of any embodiments, the one or more doses of psilocybin or an active metabolite thereof comprises a first dose of psilocybin. In some of any embodiments, the first dose is between at or about 10 mg to at or about 50 mg of psilocybin. In some of any embodiments, the first dose is at or about 15 mg of psilocybin.

In some of any embodiments, the one or more doses of psilocybin comprises a two or more doses of psilocybin.

In some of any embodiments, the one or more doses of psilocybin comprises a second dose of psilocybin. In some of any embodiments, the second dose is between at or about 20 mg to at or about 50 mg of psilocybin. In some of any embodiments, the second dose is at or about 25 mg of psilocybin. In some of any embodiments, the second dose is between at or about 10 mg to at or about 20 mg of psilocybin In some of any embodiments, the second dose is at or about 15 mg of psilocybin.

In some of any embodiments, the second dose is administered between at or about 12 days and at or about 16 days after the first dose. In some of any embodiments, the second dose is administered at or about 14 days after the first dose.

In some of any embodiments, the methods also involve providing one or more integration sessions to the subject after the one or more doses of psilocybin. In some of any embodiments, the one or more integration sessions is provided at or about 3, 4, and/or 5 weeks after administering the first dose of psilocybin. In some of any embodiments, the one or more integration sessions is provided at or about 1, 2, and/or 3 weeks after administering the second dose of psilocybin.

### BRIEF DESCRIPTION OF THE DRAWINGS

FIGS. 1A-1B show Von Frey assay (VFA) withdrawal threshold in a formalin-induced rat pain model for contralateral paw (FIG. 1A) and ipsilateral paw (FIG. 1B) after formalin administration. Baseline, BL; formalin baseline, FBL (post formalin administration).

FIGS. 2A-2B show Von Frey assay (VFA) withdrawal threshold in a formalin-induced rat pain model after formalin injection and either psilocybin or saline intravenous infusion (on Day 1). Weekly withdrawal threshold is shown for contralateral paw (FIG. 2A) and ipsilateral paw (FIG. 2B) during a 28-day analysis. Baseline, BL; formalin baseline, FBL (post formalin administration).

FIGS. 3A-3F show select treatment comparison data from the Von Frey assay (VFA) described for FIGS. 1A-B and 2A-B, including FBL and saline (FIGS. 3A and 3D), FBL and PSIL (FIGS. 3B and 3E), and saline and PSIL (FIGS. 3C and 3F) for contralateral paw (FIGS. 3A-3C) and ipsilateral paw (FIGS. 3D-3F) at day 14. Baseline, BL; formalin baseline, FBL (post formalin administration); psilocybin, PSIL.

FIGS. 4A-4D shows Hot Plate assay (HPA) withdrawal latency from a 52.5° C. hot plate in a formalin-induced rat pain model after formalin injection and either psilocybin or saline intravenous infusion (on Day 1). Weekly withdrawal latency is shown during a 28-day analysis (FIG. 4A). FIGS. 4B-4D show select treatment comparison data, including FBL and saline (FIG. 4B), FBL and PSIL (FIG. 4C), and saline and PSIL (FIG. 4D) at day 28. Baseline, BL; formalin baseline, FBL (post formalin administration); psilocybin, PSIL.

FIG. 5 shows an exemplary clinical treatment and assessment schedule in accordance with the provided embodiments.

### DETAILED DESCRIPTION

Provided herein are methods for treating or ameliorating fibromyalgia (FM) or one or more symptoms of FM, that involve administering psychedelics, such as psilocybin or a metabolite thereof, such as psilocin. In some aspects, provided herein are methods of administering and uses, such as therapeutic and prophylactic uses, of psychedelics, such as psilocybin or psilocin, for treating or ameliorating FM or one or more symptoms of FM. In some aspects, the provided methods and uses also involve psychotherapy, for example, in one or more clinical sessions, such as integration sessions.

In some aspects, the provided methods and uses also involve measuring one or more indicators before, during and/or after the treatment, including before, during and/or after the administration of the psychedelic, e.g., psilocybin or psilocin. In some aspects, the subject receiving the treatment is assessed for various indicators, such as observer-rated and subject-reported outcomes, biological and clinical indicators, and combinations thereof, before and/or after performing one or more steps of the methods or uses. In some aspects, exemplary indicators that are measured and analyzed include a fibromyalgia survey score, a pain interference assessment, a sleep assessment, a chronic pain acceptance assessment, or a clinical indicator assessment. In some aspects, the provided methods and uses leads to improvement, such as clinical improvement, or amelioration in one or more symptoms of FM. In some aspects, the improvement or amelioration can be observed based on one or more of the indicators as assessed in the subject.

In some aspects, also provided are methods for identifying or selecting subjects for treating or ameliorating FM or one or more symptoms thereof, with psychedelics, for example, in accordance with the provided therapeutic methods and uses.

Also provided herein are uses of psychedelics, such as psilocybin or psilocin, in any of the provided methods and treatments, and in the preparation of a medicament in order to carry out such methods. In some embodiments, the methods and uses thereby improve, ameliorate and/or treat FM, or one or more symptoms thereof, in the subject. In some embodiments, the provided therapeutic methods and uses, for example, involving administration of psychedelics, such as psilocybin or psilocin, in some cases in combination with psychotherapy, to a subject having, exhibiting or suffering from one or more symptoms of FM. In some aspects, the psychedelic, such as psilocybin or psilocin, is administered in an effective amount to effect improvement or amelioration in one or more symptoms of FM.

In some aspects, the treatment also involves psychotherapy. In some aspects, the provided methods and uses also involve psychotherapy, for example, in one or more clinical sessions. In some embodiments, the provided therapeutic methods and uses, for example, involving administration of psychedelics, in some cases in combination with psychotherapy, to a subject.

Fibromyalgia (FM; also known as FM syndrome) is a complex and chronic condition characterized by widespread joint and muscle pain and a variety of different symptoms. Currently available treatments and other approaches for managing fibromyalgia or one or more symptoms thereof are limited in their benefit and can have substantial burden or risks for side effects, and the duration of the effect of treatment can be limited. Accordingly, there is a need for a treatment of FM that can address one or more of the symptoms associated with FM with reduced burden or risk of side effects, and that has an extended duration. Provided are embodiments that meet such needs.

In some aspects, the provided embodiments are also based on an observation that administration of a psychedelic, such as psilocybin or psilocin, exhibited beneficial effects on pain perception in an animal model of pain that is relevant for diseases and indications associated with pain, including fibromyalgia (FM). In some aspects, as demonstrated herein, the administration of psilocybin was shown to decrease tactile pain for up to 28 days in an animal model of pain, such as a rat model of pain. In some aspects, as demonstrated herein, the administration of psilocybin was shown to decrease tactile pain for up to 28 days in an animal model of pain, such as a rat model of pain. In some aspects, the demonstrated effect of psychedelic administration on pain perception in the non-injected paw that is prolonged for weeks after administration, supports the utility of the provided embodiments in the treatment of pain related disorders, such as nociplastic pain disorders, for example, fibromyalgia (FM). In some aspects, the observations support the advantages of the provided embodiments. As psilocybin catabolism is similar in rats and humans (see, e.g., Kalberer et al., Biochem Pharmacol 1962; 11:261-9; Chen et al., J Chromatogr B Analyt Technol Biomed Life Sci, 2011; 879(25):2669-72), the results described herein of the rat pain model also provides support for administration of psilocybin or an active metabolite thereof, such as psilocin, in the treatment of pain related disorders, such as FM, in human subjects.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Methods of Treatment and Uses of Psychedelics for Fibromyalgia (FM)

Provided herein are methods of treatment, related uses, and methods for identifying or selecting an individual, such as a human subject, for treatment of fibromyalgia (FM) or one or more symptoms of FM, with by administering a psychedelic, such as psilocybin, or a metabolite thereof. In some embodiments, the individual also receives psychotherapy, for example, in one or more clinical session, in combination or in conjunction with administration of the psychedelic.

### A. Fibromyalgia

Fibromyalgia (FM; also known as FM syndrome) is a chronic condition or syndrome characterized by widespread joint and muscle pain that can include manifestation of multiple co-occurring symptoms. FM is reported to affect between 0.5 and 5% of the world population, mainly in middle-aged women (Arnold et al., Patient Educ Couns 2008; 73(1):114-20; White and Harth, Curr Pain Headache Rep 2001; 5(4):320-9). The etiology of chronic pain in FM is difficult to discern and often lacks a defined pathophysiological explanation, making diagnosis and treatment challenging. The etiology and pathophysiology of FM is not entirely understood, although inflammation in the musculoskeletal system of FM patients shows that the immune system may be involved. Genetic and environmental factors also have been reported to play a role in the etiology and pathophysiology of FM. Symptoms of FM vary widely, but include fatigue, muscle and joint stiffness, numbness, tingling, sleep disturbance, insomnia, sleep disorders, allodynia (a heightened and painful response to pressure), mood disorders, cognitive dysfunction, anxiety, depression, general sensitivity and an inability to perform routine daily tasks (Qureshi et al., Cureus 2021; 13(10):e18692). Overall, individuals with FM suffer a dramatic decrease in quality of life and these individuals have limited therapeutic options (Alciati et al., Clin Exp Rheumatol 2021; 39 Suppl 130(3):3-12).

Effective drug intervention for the hallmark conditions of FM (e.g., hyperalgesia, allodynia, sleep disorders) are limited. In some cases, individuals with FM have been treated with four main drug classes: serotonin-norephinephrine reuptake inhibitors (SNRIs), selective serotonin reuptake inhibitors (SSRIs), tricyclic anti-depressants (TCAs), and anti-epileptic drugs (AEDs) (Qureshi et al., Cureus 2021; 13(10):e18692). Three medications have been approved by the FDA for the treatment of fibromyalgia: pregabalin (Lyrica®), duloxetine (Cymbalta®), and milnacipran (Savella®). However, reports indicate that currently available medications do not provide substantial clinical benefit (Wolfe et al., Eur J Pain. 2013 April; 17(4):581-6). Individuals sometime resort to opioids, despite the lack of effect against FM, potential side effects, and many co-occurring symptoms (Fitzcharles et al., Am J Med 2011; 124(10):955-60). Currently, a large unmet need exists for treating FM and the therapeutic demand from individuals seeking relief of their debilitating symptoms.

The provided embodiments involve administration of psychedelics, such as psilocybin, for the treatment of one or more symptoms of FM. In some aspects, the one or more symptoms of FM as manifested or exhibited in a subject, are measured or assessed before beginning administration of the psychedelic, such as psilocybin. In some aspects, one or more indicators, including indicators that are associated with one or more symptoms of FM (e.g., a fibromyalgia survey score), dissociative state, treatment outcome, safety and/or one or more indicators related to the subject, are measured. In some aspects, the one or more symptoms of FM are measured or assessed, and the subject is identified or selected for treatment in accordance with the methods and uses described herein, if the subject meets one or more criteria for having one or more symptoms of FM. In some aspects, the one or more indicators and/or the one or more symptoms of FM are measured or assessed during or after administration of the psychedelic or psychotherapy. In some aspects, the one or more indicators and/or the one or more symptoms of FM measured during or after administration of the psychedelic and/or psychotherapy is assessed to measure an outcome during or after the treatment, for example, to determine the effectiveness, improvement or amelioration from the treatment. In some aspects, the one or more indicators and/or the one or more symptoms of FM are measured using any methods for measuring indicators or parameters associated with the one or more indicators and/or the one or more symptoms of FM, for example, as described in Section II.

In some aspects, provided herein is a method of treatment comprising administering psilocybin, or a metabolite thereof, and in some aspects in combination with psychotherapy to treat an individual having FM. In some embodiments, provided herein is a method of treatment comprising administering psilocybin and in some aspects in combination with psychotherapy to treat an individual diagnosed with FM. In some embodiments, provided herein is a method of treatment comprising administering psilocybin and in some aspects in combination with psychotherapy to treat an individual who displays or experiences one or more symptom of FM. In some aspects, provided herein is a method of treatment comprising administering psilocybin and in some aspects in combination with psychotherapy, wherein the severity of one or more symptom is reduced in the individual receiving treatment.

In some embodiments, provided herein is a method of treatment comprising administering psilocybin and in some aspects in combination with psychotherapy, wherein the severity of one or more symptom including but not limited to fatigue, muscle and joint stiffness, numbness, tingling, sleep disturbance, insomnia, sleep disorders, allodynia (a heightened and painful response to pressure), mood disorders, cognitive dysfunction, anxiety, depression, general sensitivity and an inability to perform routine daily tasks, is reduced in the individual receiving treatment. In some embodiments, provided herein is a method of treatment comprising administering psilocybin and in some aspects in combination with psychotherapy, wherein an improvement of one or more symptom lasts for a period of at least 3 months after treatment. In some embodiments, provided herein is a method of treatment comprising administering psilocybin and in some aspects in combination with psychotherapy, wherein an improvement of one or more symptom lasts for a period of at least 6 months after treatment. In some embodiments, provided herein is a method of treatment comprising administering psilocybin and in some aspects in combination with psychotherapy, wherein an improvement of one or more symptom lasts for a period of at least 12 months after treatment.

### B. Psychedelics and Psilocybin

In some aspects, the provided therapeutic methods and uses involve the administration of a psychedelic compound, such as psilocybin or a metabolite thereof. In some aspects, the administration of psychedelics is in conjunction with one or more clinical sessions, such as one or more preparation sessions or integration sessions, for psychotherapy. In some embodiments, the methods and uses involve orally administering one or more doses of psilocybin or psilocin to a subject suffering from one or more symptoms of FM. In some aspects, if the subject is selected or identified for treatment with psilocybin or psilocin, the subject is orally administered the one or more doses of psilocybin or psilocin.

Psychedelics (“mind manifesting” drugs) are a class of compounds that have been used in the treatment of certain mental disorders (Nutt et al., Cell 2020; 181(1):24-8), including depression and addiction. Psychedelics have a remarkable safety profile (REF) and are being considered in the context of other clinical applications. However, clinical evidence for psychedelics in treating chronic pain is lacking.

Psilocybin (3-[2-(dimethylamino) ethyl]-1H-indol-4-yl]dihydrogen phosphate; 4-phosphoroyloxy-N,N-dimethyltryptamine) is a natural product produced by numerous species of Psilocybe mushrooms, which is manufactured for clinical use to control potency and purity. It is a tryptamine derivative, and in humans the phosphate group is rapidly enzymatically cleaved in the body to produce psilocin, an agonist at a variety of serotonin receptors, the most important of which in this setting is the 5-HT2A receptor (Carhart-Harris et al., 2014, Frontiers in Human Neuroscience, 8; Nichols, 2004, Pharmacol Ther, 101(2), 131-181). Psilocybin is a prodrug, and after administration, the phosphate group is rapidly cleaved in the body by alkaline phosphatase or other non-specific esterases to produce psilocin, the active metabolite (Dinis-Oliveira R, Drug Metab Rev 2017; 49(1):84-91). Psilocin is an agonist for multiple serotonin receptors, including the 5-hydroxytryptamine (5-HT)_{2A} receptor (Carhart-Harris et al., Front Hum Neurosci 2014; 8:20; Nichols D, Pharmacol Ther 2004; 101(2):131-81). Psilocybin can induce profound changes in sensory perception, emotion, thought, and sense of self, characterized by marked alterations in all mental functions, including perception, mood, volition, cognition and self-experience (Geyer & Vollenweider, Trends Pharmacol Sci. 2008; 29(9):445-53; Studerus et al., J Psychopharmacol. 2011; 25(11):1434-52). These profound changes are often referred to as mystical-type experiences. Measures of mystical-type experience occurring during psilocybin treatment have been repeatedly observed to predict later effects on behavior and emotions, including reductions in depressive and anxious symptoms (Griffiths et al., J Psychopharmacol. 2016; 30(12):1181-1197; Maclean et al., J Psychopharmacol. 2011; 25(11):1453-1461; Ross et al., J Psychopharmacol. 2016; 30(12):1165-1180). Psilocybin has similar effects to dimethyltryptamine (DMT), lysergic acid diethylamide (LSD) and mescaline. These agents produce psychoactive effects that have been characterized as forming an intense dream-like state with colorful visual illusions, changes in auditory, tactile, olfactory, gustatory, and kinesthetic perceptions, altered perceptions of time and space, changes in body image, and sensations including ego dissolution, and intense mood changes ranging from feelings of wonder and bliss to sadness and grief.

In some aspects, psilocybin induces profound changes in sensory perception, emotion, thought, and sense of self, characterized by marked alterations in all mental functions, including perception, mood, volition, cognition and self-experience (Geyer & Vollenweider, Trends Pharmacol Sci. 2008; 29(9):445-53; Studerus et al., J Psychopharmacol. 2011; 25(11):1434-52). These profound changes are often referred to as mystical-type experiences which have been repeatedly observed to predict later effects on behavior and emotions, including reductions in depressive and anxious symptoms (Griffiths et al., J Psychopharmacol. 2016; 30(12):1181-1197; Maclean et al., J Psychopharmacol. 2011; 25(11):1453-1461; Ross et al., J Psychopharmacol. 2016; 30(12):1165-1180). In some aspects, the action of psilocybin has been primarily associated with activation of the 5-HT2A receptor.

Oral psilocybin has about a 50% bioavailability and psilocin is detectable in plasma within 20 minutes of administration of the parent compound (Brown et al. Clin Pharmacokinet. 2017; 56(12):1543-1554; Pharm Acta Helv. 1997; 72(3):175-84). The half-life of psilocin in blood is 2-3 hours. In general, onset of noticeable psychoactive effects occurs within one hour and peaks at about two hours after a dose. Loss of noticeable effects typically occurs around six hours after administration. Based on this time course, observation in the clinical trial setting until 8 hours after dosing. Furthermore, exposure following a 25 mg oral dose is associated with both near-maximal occupancy of neocortical serotonin 5-HT2A receptors and subjective intensity ratings of elements of the psychedelic experience that have been repeatedly associated with longer-term therapeutic benefits (Madsen et al., Neuropsychopharmacology. 2019; 44(7): 1328-1334).

Non-clinical studies have reported that, similar to humans, when psilocybin is administered orally to rats it is rapidly dephosphorylated to psilocin in the intestinal mucosa by alkaline phosphatase and a nonspecific esterase, with approximately 50% of the total volume of psilocin absorbed from the digestive tract (Kalberer et al., Biochem Pharmacol. 1962; 11:261-9). Maximum plasma levels are achieved after approximately 90 minutes (Chen et al., J Chromatogr B Analyt Technol Biomed Life Sci, 2011; 879(25):2669-72). When administered systemically (i.e., bypassing the gut), initial psilocybin metabolism is performed by tissue phosphatases, with in vitro studies indicating the kidneys as being among the most active metabolic organs (Horita & Weber, Biochem. Pharmacol. 1961; 7(1): 47-54). Across species tested, the highest levels of psilocin were found in the neocortex, hippocampus, and thalamus (Hopf & Eckert, Act Nerv Super (Praha) 1974; 16(1):64-6). In certain studies, psilocybin showed a decrease in symptomatic response in indications including obsessive compulsive disorder (OCD), substance use disorder, depression, and anxiety. Overall, psilocybin has been well tolerated at the doses examined in clinical studies.

Studies in rats demonstrated that psilocybin catabolism is similar to humans and approximately 50% of the total psilocin is absorbed from the digestive tract (Kalberer et al., Biochem Pharmacol 1962; 11:261-9), with maximum plasma levels achieved approximately 90 minutes post-administration (Chen et al., J Chromatogr B Analyt Technol Biomed Life Sci, 2011; 879(25):2669-72). In some aspects, when administered systemically (i.e., bypassing the gut), initial psilocybin metabolism is performed by tissue phosphatases, with in vitro studies indicating the kidneys as being among the most active metabolic organs (Horita & Weber, Biochem Pharmacol 1961; 7(1):47-54). Further, the highest levels of psilocin were consistently found in the neocortex, hippocampus, and thalamus (Hopf & Eckert, Act Nerv Super (Praha) 1974; 16(1):64-6).

In the clinical environment, psilocybin administration has been reported to improve certain symptoms of cancer-related psychiatric stress, depression and anxiety, nicotine and alcohol addiction, obsessive compulsive disorder (OCD) (Nutt et al., Cell 2020; 181(1):24-8), and anorexia nervosa, a condition where intrusive thoughts drive maladaptive and life-threatening behavior and in which treatment resistance is common (Foldi et al., 2020, Frontiers in Neuroscience, 14). Psilocybin was well tolerated at the doses examined in clinical studies. Further, a single dose of psilocybin was shown to improve patient-reported depression scores after 1 week, with these improvements persisting for up to 6 months (Carhart-Harris et al., Psychopharmacol 2018; 235(2):399-408). Psilocybin has since been given a fast-track designation by the FDA for depression.

### 1. Dosing

In some aspects, provided herein is a method of treatment comprising psilocybin or psilocin dosing regimens to treat an individual, such as a human subject, having fibromyalgia (FM) or suffering from or exhibiting one or more symptoms of FM. In some aspects, provided herein are psilocybin or psilocin dosing regimens to treat an individual who has experienced one or more symptoms of FM. In some aspects, provided herein are psilocybin or psilocin dosing regimens to treat an individual diagnosed with FM. In some embodiments, a method for treating an individual that exhibits or is suffering from one or more symptoms of FM comprises administering to the subject one or more therapeutically effective dose(s) of psilocybin or psilocin.

In some aspects, one or more doses of psilocybin is administered to the individual (such as a subject having FM or suffering from or exhibiting one or more symptoms of FM, or a subject identified or selected for treatment). In some aspects, a first dose and a second dose of psilocybin are administered. In some aspects, the psilocybin is administered orally. In some aspects, the psilocybin is dosed intravenously.

In some aspects, one or more doses of psilocin is administered to the individual (such as a subject having FM or suffering from or exhibiting one or more symptoms of FM, or a subject identified or selected for treatment). In some aspects, a first dose and a second dose of psilocin are administered. In some aspects, the psilocin is administered orally. In some aspects, the psilocin is dosed intravenously.

In some cases, the first dose of psilocybin may not produce a full psychedelic effect. In some aspects, the first dose can help familiarize the subject with the administration of the psychedelic and its effects. In some aspects, the second dose, which, in some cases is higher than the first dose, is a therapeutically effective dose or produces a therapeutic effect. In some aspects, the two-dose treatment regimen also allows the assessment of the suitability of the subject for receiving a treatment involving administration of psychedelics for FM, and can be used to identify and/or select a subject for treatment in accordance with the provided methods and uses. In some embodiments, the first dose is used to assess the suitability of the subject for receiving a treatment in accordance with the provided embodiments, for example, for receiving a second, higher dose. In some aspects, the first dose can be used to assess adverse effects or side effects that may be associated with the administration of the psychedelic. In some aspects, the two-dose treatment regimen also allows assessment to reduce the likelihood of unexpected adverse events in subjects who may have higher bioavailability and/or higher rates of conversion to psilocin.

Oral psilocybin has about a 50% bioavailability and psilocin is detectable in plasma within 20 minutes of administration of the parent compound (Brown et al. Clin Pharmacokinet. 2017; 56(12):1543-1554; Pharm Acta Helv. 1997; 72(3):175-84). The half-life of psilocin in blood is 2-3 hours. Onset of noticeable psychoactive effects occurs within one hour and peaks at about two hours after a dose. Loss of noticeable effects typically occurs around six hours after administration. In one report, peak plasma levels of psilocin after a single oral administration of psilocybin at 0.225 mg/kg occurred after 105+/−37 min showing an average concentration of 8.2+/−2.8 ng psilocin/ml plasma and estimates for the absolute bioavailability of psilocin after oral administration of psilocybin are 52.7+/−20% (Hasler et al, Pharm Acta Helv. 1997 June; 72(3):175-84). In certain reports, following a 25 mg oral dose was associated with both near-maximal occupancy of neocortical serotonin 5-HT_{2A} receptors and subjective intensity ratings of elements of the psychedelic experience that have been repeatedly associated with longer-term therapeutic benefits (Madsen et al., Neuropsychopharmacology 2019; 44(7):1328-34).

In some embodiments, one or more doses of the psychedelic, such as psilocybin or an active metabolite thereof, is administered to the subject. In some embodiments, one, two, three, four or five doses is administered to the subject. In some embodiments, one dose is administered. In some embodiments, two or more doses is administered to the subject. In some aspects, two doses are administered.

In some embodiments, the non-initial, subsequent dose can be any dose of the psychedelic, such as psilocybin or an active metabolite thereof, that is administered after the initial or first dose, wherein at least two doses are administered to a subject. In some embodiments, the non-initial, subsequent dose can be any dose that is administered after the initial or first dose, wherein two or more doses are administered to a subject. In some embodiments, the non-initial subsequent dose can be a second, a third, or a fourth dose of psilocybin. In some embodiments, the subject receives a subsequent dose as needed. In some embodiments, the subject receives subsequent intermittent doses as needed.

In some embodiments, when two or more doses of the psychedelic, such as psilocybin or a metabolite thereof, is administered, the dosing interval between a previous dose and a subsequent dose, such as a first dose and a second dose, is at or about 1 week to 4 weeks. In some embodiments, a second dose is administered at or about 1 week, about 2 weeks, about 3 weeks, about 4 weeks, after the first dose. In some aspects, the dosing interval, for example the time between the first dose and the second dose, is at or about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, about 21 days, 22 days, 23 days, 24 days, 25 days, 26 days, 27 days, or 28 days. In some embodiments, a second dose is administered at or about at or about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, about 21 days, 22 days, 23 days, 24 days, 25 days, 26 days, 27 days, or 28 days after the first dose.

In some embodiments, a dose of the psychedelic, such as psilocybin or a metabolite thereof, is administered to a subject, such as a subject having FM. In some aspects, the dose of the psilocybin is a therapeutically effective dose. In some aspects, the dose of psilocybin is effective to and/or sufficient to induce a dissociative state. In some aspects, the therapeutically effective dose of psilocybin is based on the route of administration. In some aspects, the dose of the psilocin is a therapeutically effective dose. In some aspects, the dose of psilocin is effective to and/or sufficient to induce a dissociative state. In some aspects, the therapeutically effective dose of psilocin is based on the route of administration.

In some embodiments, the dose is a flat or a fixed dose. In some embodiments, the dose to be administered is between at or about 5 mg and at or about 50 mg. In some embodiments, the dose to be administered is at or about 5 mg, 10 mg, 15 mg, 20 mg, 25 mg, 30 mg, 35 mg, 40 mg, 45 mg, or 50 mg. In some aspects, the dose is administered orally. In some aspects, for oral administration, the dose to be administered is between at or about 5 mg and at or about 50 mg, such as at or about 5 mg, 10 mg, 15 mg, 20 mg, 25 mg, 30 mg, 35 mg, 40 mg, 45 mg, or 50 mg. In some embodiments, the dose to be administered, such as by an oral administration, is at or about 25 mg. In some embodiments, the dose to be administered, such as by an oral administration, is at or about 50 mg.

In some aspects, for oral administration, the first dose to be administered is between at or about 5 mg and at or about 50 mg, such as at or about 5 mg, 10 mg, 15 mg, 20 mg, 25 mg, 30 mg, 35 mg, 40 mg, 45 mg, or 50 mg. In some embodiments, the first dose to be administered, such as by an oral administration, is at or about 25 mg. In some embodiments, the first dose to be administered, such as by an oral administration, is at or about 50 mg. In some aspects, for oral administration, the first dose of psilocybin to be administered is between at or about 5 mg and at or about 50 mg, such as at or about 5 mg, 10 mg, 15 mg, 20 mg, 25 mg, 30 mg, 35 mg, 40 mg, 45 mg, or 50 mg. In some embodiments, the first dose of psilocybin to be administered, such as by an oral administration, is at or about 25 mg. In some embodiments, the first dose of psilocybin to be administered, such as by an oral administration, is at or about 50 mg.

In some embodiments, the dose determined according to body weight. In some embodiments, the first dose is determined according to body weight. In some aspects, exemplary dose based on body weight include between 0.1 mg/kg, at or about 0.15 mg/kg, at or about 0.25 mg/kg, at or about 0.35 mg/kg, at or about 0.45 mg/kg, at or about 0.55 mg/kg, at or about 0.65 mg/kg, at or about 0.75 mg/kg, at or about 0.85 mg/kg, at or about 0.95 mg/kg, or at or about 1.0 mg/kg. In some embodiments, a dose of at or about 25 mg is administered to a subject having a body weight of less than 100 kg (≤100 kg). In some embodiments, a dose of at or about 30 mg is administered to a subject having a body weight of greater than 100 kg and less than or equal to 117 kg (>100 kg and ≤117 kg). In some embodiments, a dose of at or about 35 mg is administered to a subject having a body weight of greater than 117 kg (>117 kg).

In some embodiments, a dose subsequent to the first dose is determined according to body weight. In some embodiments, the subsequent dose is increased by between at or about 0.1 mg/kg to at or about 1 mg/kg, such as at or about 0.1 mg/kg, at or about 0.15 mg/kg, at or about 0.25 mg/kg, at or about 0.35 mg/kg, at or about 0.45 mg/kg, at or about 0.55 mg/kg, at or about 0.65 mg/kg, at or about 0.75 mg/kg, at or about 0.85 mg/kg, at or about 0.95 mg/kg, or at or about 1.0 mg/kg, relative to the first, initial dose. In some embodiments, the subsequent dose is increased by at or about 0.15 mg/kg, at or about 0.25 mg/kg, at or about 0.35 mg/kg, at or about 0.45 mg/kg, at or about 0.55 mg/kg, at or about 0.65 mg/kg, at or about 0.75 mg/kg, at or about 0.85 mg/kg, and at or about 0.95 mg/kg relative to the first, initial dose.

In some embodiments, a subsequent dose of at or about 30 mg is administered to a subject having a body weight of less than 80 kg (≤80 kg). In some embodiments, a subsequent dose of at or about 35 mg is administered to a subject having a body weight of greater than 80 kg and less than or equal to 90 kg (>80 kg and ≤90 kg). In some embodiments, a subsequent dose of at or about 40 mg is administered to a subject having a body weight of greater than 90 kg and less than or equal to 100 kg (>90 kg and ≤100 kg). In some embodiments, a subsequent dose of at or about 45 mg is administered to a subject having a body weight of greater than 100 kg and less than or equal to 112 kg (>100 kg and ≤112 kg). In some embodiments, a subsequent dose of at or about 50 mg is administered to a subject having a body weight of greater than 112 kg (>112 kg).

In some aspects, the first dose is between at or about 10 mg to at or about 20 mg, such as oral psilocybin. In some aspects, the first dose is at or about 10 mg, 11 mg, 12 mg, 13 mg, 14 mg, 15 mg, 16 mg, 17 mg, 18 mg, 19 mg, or 20 mg, such as oral psilocybin. In some embodiments, the first dose is at or about 15 mg, such as oral psilocybin.

In some embodiments, the second dose is higher than the first dose. In some aspects, the second dose is between at or about 20 mg to at or about 30 mg, such as oral psilocybin. In some aspects, the second dose is at or about 20 mg, 21 mg, 22 mg, 23 mg, 24 mg, 25 mg, 26 mg, 27 mg, 28 mg, 29 mg, or 30 mg, such as oral psilocybin. In some embodiments, the second dose is at or about 25 mg, such as oral psilocybin.

In some embodiments, the second dose is identical to the first dose In some aspects, the second dose is between at or about 10 mg to at or about 20 mg, such as oral psilocybin. In some aspects, the second dose is at or about 10 mg, 11 mg, 12 mg, 13 mg, 14 mg, 15 mg, 16 mg, 17 mg, 18 mg, 19 mg, or 20 mg, such as oral psilocybin. In some embodiments, the second dose is at or about 15 mg, such as oral psilocybin. In some aspects, the second dose is identical to the first dose if it is determined that a higher dose would not be advantageous or may not provide further benefit.

In some aspects, the second dose is administered about one, two, three or four weeks after the first dose. In some aspects, the second dose is administered between at or about 12 days and at or about 16 days after the first dose. In some aspects, the second dose is administered at or about 12 days, 13 days, 14 days, 15 days, or 16 days, after the first dose. In some embodiments, the second dose is administered at or about 14 days after the first dose.

In some aspects, psilocybin is administered orally. In some embodiments, psilocybin is administered in tablet form. In some embodiments, psilocybin is administered in capsule form. In some embodiments, one or more psilocybin 5 mg capsule(s) is administered to meet the desired dosing level in a subject. In some embodiments, one or more psilocybin 25 mg capsule(s) is administered to meet the desired dosing level in a subject. In some embodiments, psilocybin is administered capsules comprising one or more of gelatin, hydroxypropyl methyl cellulose (HPMC) or hypromellose, and pullulan. In some embodiments, psilocybin is administered capsules comprising hydroxypropyl methyl cellulose (HPMC) or hypromellose.

In some embodiments, wherein the psychedelic, such as psilocybin or a metabolite thereof, is administered to a subject more than once, or repeatedly dosed, the amount of the initial dose and the amount of the one more subsequent dose(s) are the same. In some embodiments, wherein the psychedelic, such as psilocybin or a metabolite thereof, is administered to a subject more than once, or repeatedly dosed, the amount of the initial dose and the amount of the one more subsequent dose(s) are different. In some embodiments, wherein the psychedelic, such as psilocybin or a metabolite thereof, is administered to a subject more than once, or repeatedly dosed, the amount of the initial dose is less than the amount of the one more subsequent dose(s). In some embodiments, wherein the psychedelic, such as psilocybin or a metabolite thereof, is administered to a subject more than once, or repeatedly dosed, the amount of the initial dose is greater than the amount of the one more subsequent dose(s).

### C. Psychological Support

In some embodiments, the provided methods and uses involve providing psychological support, e.g., psychotherapy or mental health support, in addition to or in conjunction with administration of the psychedelic, such as psilocybin or psilocin. In some aspects, the methods or uses involve administration of the psychedelic and psychological support or mental health support, e.g., psychotherapy, to an individual, such as a human subject that exhibits or is suffering from one or more symptoms of FM. In some aspects, the methods or uses involve administration of the psychedelic and psychotherapy to an individual that exhibits or is suffering from one or more symptoms of FM. In some aspects, the methods or uses involve administration of the psychedelic and talk therapy to an individual that exhibits or is suffering from one or more symptoms of FM. In some embodiments, psychological support or mental health support occurs in-person and/or remotely, such as by phone or video conference. In some embodiments, psychotherapy occurs in-person and/or remotely, such as by phone or video conference. In some embodiments, talk therapy occurs in-person and/or remotely, such as by phone or video conference.

In some embodiments, psychological support comprises psychotherapy and/or talk therapy. In some embodiments, psychological support is therapist-led or led by a person or persons other than the individual. In some embodiments, psychological support is self-administered. In some embodiments, psychological support is therapist-led and self-administered. In some embodiments, psychological support precedes administration of the psychedelic, such as during a preparation session. In some embodiments, psychological support does not precede administration of the psychedelic. In some embodiments, psychological support accompanies administration of the psychedelic. In some embodiments, psychological support follows or occurs after administration of the psychedelic, such as during an integration session. In some aspects, the initial session of psychological support involves a therapist, and additional sessions does not involve a therapist.

In some aspects, the provided embodiments involve providing psychological support and/or psychotherapy in one or more sessions, such as one or more preparation sessions or integration sessions. In some embodiments, psychological support is facilitated in one or more sessions. In some embodiments, psychological support and/or psychotherapy is provided prior to administration of the psychedelic, such as during a preparation session. In some embodiments, psychological support accompanies administration of the psychedelic. In some embodiments, psychological support and/or psychotherapy is provided subsequent to administration of the psychedelic, such as during an integration session. In some embodiments, psychological support and/or psychotherapy is provided prior to, during administration, and subsequent to administration of the psychedelic, wherein the psychedelic is administered at least once, at least twice, at least three times, or at least four times.

In some embodiments, psychological support and/or psychotherapy is provided conducted in single-subject sessions, wherein one subject meets with one or more therapist(s). In some embodiments, psychological support and/or psychotherapy is provided conducted in single-subject sessions, wherein one subject meets with two or more therapist(s) In some embodiments, psychological support and/or psychotherapy is provided conducted in group sessions, wherein more than one subject meets with one or more therapist(s). In some embodiments, one or more of the subject's family members or friends may be present at the pre-administration psychological support session(s). In some embodiments, the one or more psychological support sessions conducted prior to administration of the psychedelic is conducted in person or remotely, such as by phone or video conference.

In some embodiments, psychological support conducted prior to the psychedelic administration comprises the pursuit of one or more goals and/or objectives. In some embodiments, the one or more goal(s) and/or objective(s) of the pre-administration session comprise(s) establishing a therapeutic alliance between individual and therapist, answering the individual's questions and addressing any concerns, demonstrating and practicing self-directed inquiry and experiential processing. In some embodiments, the pre-administration psychological support sessions, such as the one or more preparation sessions, include discussion of the individual's life history, current situation in life, as well as the individual's understanding, expectations, and intentions for the administration sessions. In some embodiments, the pre-administration psychological support sessions comprises discussion of the reliable and/or the potential effects of the psychedelic. In some embodiments, the pre-administration psychological support session comprises developing rapport with the therapist, learning about fibromyalgia, and building comfort and understanding regarding the physical space and the administration setting and sessions. In some embodiments, the pre-administration psychological support session comprises preparing the individual for the psychedelic administration by practicing relevant therapeutic techniques to reduce avoidance and anxiety, eliciting relevant therapeutic goals, building rapport, and/or establishing therapeutic alliance. In some embodiments, the pre-administration psychological support session comprises listening to the individual's narrative of fibromyalgia, comprising aspects of chronic pain, sleep disturbances, emotional or cognitive impairment, and treatment history. In some embodiments, the pre-administration psychological support session comprises psychoeducation regarding the psychedelic experience and Acceptance and Commitment Therapy (ACT).

In some embodiments, breathing exercises meant to promote calm and/or ease anxiety may be demonstrated and/or practiced. In some embodiments, breathing exercise comprise instructing the individual to focus on their breath and/or sensations associated with the breath throughout the body. In one example, the individual may be instructed to breathe in for a count of four, to hold their breath for a moment, and then to breathe out for a count of about eight. In some embodiments, the therapist and individual may discuss the most helpful ways to support in case of emotional distress during the psychedelic session. In some embodiments, the therapist may assist the individual in setting a motivation for the psychedelic session. In some embodiments, the individual is given access (e.g., online access) to materials concerning the safety and mechanism of action of the psychedelic.

In some embodiments, psychological support is carried out in one or more sessions. In some embodiments, the methods or uses involve psychological support prior to administration of the psychedelic, such as during a preparation session. In some embodiments, psychological support accompanies administration of the psychedelic. In some embodiments, the methods or uses involve psychological support subsequent to administration of the psychedelic, such as during an integration session. In some embodiments, the methods or uses involve providing psychological support prior to, during administration, and subsequent to administration of the psychedelic, wherein the psychedelic is administered at least once, at least twice, at least three times, or at least four times.

In some embodiments, the methods or uses involve providing one or more sessions of psychological support, such as one or more sessions of psychotherapy, prior to a first or an initial dose of the psychedelic, such as one or more preparation session(s). In some embodiments, the methods or uses involve two or more preparation sessions, such as three, four or five sessions, of psychological support prior to a first or an initial dose of the psychedelic. In some embodiments, the methods or uses involve two preparation sessions of psychological support prior to a first or an initial dose of the psychedelic.

In some embodiments, the individual receives at least one psychological support session before administration (e.g., pre-administration) of the psychedelic. In some embodiments, the methods or uses involve a pre-administration psychological support session held at or about 1 month, 4 weeks, 3 weeks, 2 weeks, or 1 week prior to administration of the psychedelic. In some embodiments, the methods or uses involve a pre-administration psychological support session held about two weeks prior to administration of the psychedelic, such as a first or an initial dose of the psychedelic. In some embodiments, the methods or uses involve a pre-administration psychological support session held about one week prior to administration of the psychedelic, such as a first or an initial dose of the psychedelic. In some embodiments, the methods or uses involve a pre-administration psychological support session held at or about 14, 13, 12, 11, 10, 9, 8, 7, 6, 5, 4, 3, 2, or 1 day prior to administration of the psychedelic, such as a first or an initial dose of the psychedelic. In some embodiments, the methods or uses involve a pre-administration psychological support session held about 11 days prior to administration of the psychedelic, such as a first or an initial dose of the psychedelic. In some embodiments, the methods or uses involve a pre-administration psychological support session held about 4 days prior to administration of the psychedelic, such as a first or an initial dose of the psychedelic. In some embodiments, the methods or uses involve a pre-administration psychological support session held one day prior to administration of the psychedelic. In some embodiments, one or more psychological support sessions is conducted prior to the initiation administration of the psychedelic, such as prior to the first or initial dose of the psychedelic.

In some embodiments, the methods or uses involve two psychological support sessions prior to a first or an initial dose of the psychedelic, such as two preparation sessions. In some embodiments, the preparation sessions are held within about one or two weeks prior to administration of the psychedelic. In some embodiments, the first preparation session is held about 14, 13, 12, 11, 10, 9, 8, 7, 6, 5, 4, 3, 2, or 1 day prior to administration of the psychedelic, such as a first or an initial dose of the psychedelic. In some embodiments, the first preparation session is held at or about two weeks or one week prior to administration of the psychedelic, such as a first or an initial dose of the psychedelic. In some embodiments, the second preparation session is held about 4, 3, 2, or 1 days prior to administration of the psychedelic, such as a first or an initial dose of the psychedelic. In some embodiments, the second preparation session is held about 4 days prior to administration of the psychedelic, such as a first or an initial dose of the psychedelic.

In some embodiments, the methods or uses involve providing psychological support during or concurrently with the psychedelic administration session. In some embodiments, the methods or uses involve administering the psychedelic to an individual in a controlled environment, wherein the individual is provided with psychological support. In some embodiments, the methods or uses involve administering one or more dose(s) of the psychedelic (e.g., an initial and one or more subsequent doses, or a first and a second dose) to the individual in the controlled environment, wherein the individual is provided with psychological support.

In some embodiments, the methods or uses involve administering a dose of the psychedelic to the individual in the controlled environment, wherein the individual is provided with psychological support. In some embodiments, the methods or uses involve administering one or more dose(s) of the psychedelic (e.g., an initial and one or more subsequent doses, or a first and a second dose) of the psychedelic to the individual in the controlled environment, for example, wherein the individual is provided with psychological support. In some embodiments, the psychedelic is administered in a setting under the guidance of a psychological support provider, such as a therapist. In some aspects, the psychological support provider, such as a therapist, is present during the administration session and provide support for the individual's physical and emotional needs during the administration session. In some aspects, a physician is on call and available nearby during the administration session, such as in the first 1, 2, 3, or 4 hours of and/or until the peak effects of the psychedelic have abated. In some aspects, a physician is on call and available nearby for consultation, throughout the administration session.

In some aspects, the psychedelic is administered in a comfortable setting. In some embodiments, the individual receives the psychedelic administration lying down on comfortable couch or bed, wearing eyeshades, and listening to music through headphones. In some embodiments, the individual is encouraged to direct their attention inward. In some aspects, during the administration session, the psychological support provider, such as a therapist, measures or assesses one or more observer-reported parameters and/or indicators, such as any described herein, for example, in Section II. In some aspects, during the administration session, the psychological support provider, such as a therapist, measures or assesses the presence/intensity of behaviors, signs, and reported symptoms, such as peacefulness, yawning, nausea/vomiting, quantity of speech, anxiety, sleepiness, crying, restlessness, visual changes, euphoria, and feelings of unreality.

In some embodiments, the methods or uses involve one or more sessions of psychological support post-administration of the psychedelic. In some embodiments, the methods or uses involve one or more sessions of psychological support after a first or an initial dose of the psychedelic. In some embodiments, the methods or uses involve 2, 3, 4, 5, 6, 7, 8, 9, 10 or more sessions of psychological support after a first or an initial dose of the psychedelic.

In some embodiments, the methods or uses involve one or more sessions of psychological support after a non-initial, subsequent dose of the psychedelic. In some embodiments, the methods or uses involve one or more sessions of psychological support after a non-initial, subsequent dose of the psychedelic. In some embodiments, the methods or uses involve 2, 3, 4, 5, 6, 7, 8, 9, 10 or more sessions of psychological support after a non-initial, subsequent dose of the psychedelic. In some embodiments, the methods or uses involve 2, 3, 4, 5, 6, 7, 8, 9, 10 or more sessions of psychological support after a non-initial, subsequent dose of the psychedelic. In some embodiments, the methods or uses involve at least 3 sessions of psychological support after a non-initial, subsequent dose of the psychedelic, such as a second dose of the psychedelic.

In some embodiments, the non-initial, subsequent dose can be any dose of the psychedelic that is administered after the initial or first dose, wherein at least two doses are administered to an individual that exhibits or is suffering from one or more symptoms of FM. In some embodiments, the non-initial, subsequent dose can be any dose of the psychedelic that is administered after the initial or first dose, wherein two or more doses of the psychedelic are administered to an individual that exhibits or is suffering from one or more symptoms of FM. In some embodiments, the non-initial subsequent dose is a second, a third, or a fourth dose of the psychedelic. In some embodiments, the non-initial subsequent dose is a second dose of the psychedelic.

In some embodiments, 3 psychological support sessions, such as 3 integration sessions are provided subsequent to administration of a subsequent dose, such as a second dose, of psychedelic. In some embodiments, the 3 integration sessions are held within about 3 weeks after administration of psychedelic, such as a second dose of psychedelic. In some embodiments, the 3 integration sessions are held within 3 weeks after administration of psychedelic, approximately once weekly. In some embodiments, the first of 3 integration sessions is held about 1 week after the psychedelic administration session. In some embodiments, the first of 3 integration sessions is held about 5, 6, 7, 8, or 9 days after the psychedelic administration session. In some embodiments the second of 3 integration sessions is held about 2 weeks after the psychedelic administration session. In some embodiments the second of 3 integration sessions is held about 11, 12, 13, 14, 15, or 16 days after the psychedelic administration session. In some embodiments the third of 3 integration sessions is held about 3 weeks after the psychedelic administration session. In some embodiments the third of 3 integration sessions is held about 18, 19, 20, 21, 22, 23 or 24 days after the psychedelic administration session.

In some embodiments, the one or more post-administration session or integration session comprises eliciting a complete narrative of the individual's experience during the psychedelic session. In some embodiments, the one or more post-administration session or integration session comprises focused ACT-based clinical activity related to contacting the present moment, defusion, or acceptance based on clinical formulation. In some embodiments, the one or more post-administration session or integration session comprises reviewing and reflecting upon the individual's the psychedelic experience, including any emotional, mental, or lifestyle changes that followed the dosing session.

In some embodiments, the one or more post-administration session or integration session is held under guidance of a psychological support provider, such as a therapist. In some aspects, in the one or more post-administration session or integration session involves discussion on integration, helping subjects gain insight and understanding from the experience from administration of the psychedelic.

### 1. Psychological Support Prior to Administration of the Psychedelic

In some embodiments, one or more sessions of psychological support and/or psychotherapy is provided prior to a first or an initial dose of the psychedelic (such as psilocybin or a metabolite thereof), such as one or more preparation session(s). In some embodiments, two or more sessions of psychological support, such as three, four or five or more sessions of psychological support and/or psychotherapy, is provided prior to a first or an initial dose of the psychedelic.

In some embodiments, the subject participates in at least one psychological support session before administration, e.g., pre-administration, of the psychedelic. In some embodiments, a pre-administration psychological support session is provided at or about 1 month or less, such as at or about four weeks, three weeks, two weeks or one week prior to administration of the psychedelic. In some embodiments, the subject participates in at least one psychological support session before administration, e.g., pre-administration, of the psychedelic. In some embodiments, a pre-administration psychological support session is provided at or about 1 week or less, such as at or about seven days, six days, five days, four days, three days, two days, or one day prior to administration of the psychedelic.

In some embodiments, one or more psychological support sessions is conducted prior to administration of the psychedelic. In some aspects, two psychological support sessions are provided prior to administration of the psychedelic. In some embodiments, two psychological support sessions are provided prior to a first the psychedelic administration session, such as two preparation sessions. In some aspects, two psychological support sessions are provided prior to administration of the psychedelic, with the first session at or about 1 week prior to administration of the psychedelic and at or about 1 to 3 days prior to the administration of the psychedelic, such as at or about 2 days prior to the administration of the psychedelic.

In some embodiments, the one or more psychological support sessions conducted prior to administration of the psychedelic is conducted in person or remotely, such as by phone or video conference.

In some embodiments, breathing exercises meant to promote calm and/or ease anxiety may be provided, demonstrated and/or practiced. In some embodiments, breathing exercise comprise instructing the subject to focus on their breath and/or sensations associated with the breath throughout the body. In one example the subject may be instructed to breathe in for a count of four, to hold their breath for a moment, and then to breathe out for a count of eight. In some embodiments, the therapist and subject may discuss the most helpful ways to support in case of emotional distress during the psychedelic session. In some embodiments, the therapist may assist the subject in setting a motivation for the psychedelic session. In some embodiments, the subject is given access (e.g., online access) to materials concerning the safety and mechanism of action of the psychedelic. In some embodiments, the subject may listen to programmed music, or the therapist provides programmed music during the one or more psychological support sessions.

In some embodiments, psychological support and/or psychotherapy conducted prior to the psychedelic administration comprises the pursuit of one or more goals and/or objectives. In some embodiments, the one or more goal(s) and/or objective(s) of the pre-administration session comprise(s) establishing a therapeutic alliance between subject and therapist, answering the subject's questions and addressing any concerns, demonstrating and practicing self-directed inquiry and experiential processing. In some embodiments, the pre-administration psychological support sessions comprise discussion of the reliable and/or the potential effects of the psychedelic.

In some embodiments, the pre-administration psychological support session comprises preparing the subject for the psychedelic administration by practicing relevant therapeutic techniques to reduce avoidance and anxiety, eliciting relevant therapeutic goals, building rapport, and/or establishing therapeutic alliance. In some embodiments, the pre-administration psychological support session comprises preparing the subject for the psychedelic administration by practicing relevant therapeutic techniques to build a rapport and trust between the individual and the therapist, for example, such that the therapist can guide the individual through the experience. In some embodiments, the pre-administration psychological support session includes listening to the subject's narrative of pain and others, such as to understand patterns of psychological inflexibility that are most prominent. In some embodiments, the pre-administration psychological support session comprises psychoeducation regarding the psychedelic experience and Acceptance and Commitment Therapy (ACT). In some embodiments, at one or more, two or more, or three or more psychological support sessions are conducted prior to administration of the psychedelic.

### 2. Psychological Support Accompanying Administration of the Psychedelic

In some embodiments, psychological support is provided during the administration session. In some aspects, the provided embodiments involve administering the psychedelic, such as psilocybin, to a subject in a controlled environment, wherein the subject is provided with psychological support. In some aspects, the provided embodiments involve administering one or more dose(s) of psilocybin, e.g., an initial and one or more subsequent doses, or a first and a second dose, to the subject in the controlled environment, wherein the subject is provided with psychological support. In some embodiments, the subject is administered an initial psychological support sessions led by a therapist in person at the site of administration, and additional or subsequent support sessions are provided remotely or are self-administered.

In some embodiments, a therapeutically effective dose of the psychedelic is administered to the subject in the controlled environment, wherein the subject is provided with psychological support. In some embodiments, one or more dose(s) of the psychedelic, e.g., an initial and one or more subsequent doses, or a first and a second dose, of the psychedelic is administered to the subject in the controlled environment, wherein the subject is provided with psychological support. In some embodiments, a psychological support provider does not provide significant Acceptance and Commitment Therapy (ACT) interventions or feedback. In some embodiments, ACT-based clinical formulation continues as the psychological support provider listens to emergent narratives and notes instances of psychological flexibility and inflexibility, especially present moment awareness, avoidance, and values. In some embodiments, a psychological support provider listens to the individual during the dosing.

### 3. Psychological Support Subsequent to Administration of the Psychedelic

In some embodiments, one or more sessions of psychological support and/or psychotherapy is provided post-administration of one or more doses the psychedelic (such as psilocybin or a metabolite thereof), such as the first dose of the psychedelic. In some embodiments, the one or more sessions of psychological support and/or psychotherapy is provided as one or more integration sessions. In some aspects, the one or more integration sessions to the subject after emergence from the dissociative state from the one or more doses of the psychedelic.

In some embodiments, one or more integration sessions, such as two, three or four or more integration sessions is provided after each dose. In some embodiments, two integration sessions is provided after each dose. In some aspects, two integration sessions are provided after the first dose. In some aspects, two integration sessions are provided after a subsequent dose, such as a second dose. In some aspects, the integration sessions include psychological support, psychotherapy and/or talk therapy. In some embodiments, psychological support is therapist-led or led by a person or persons other than the subject. In some embodiments, psychological support is self-administered. In some embodiments, aspects of psychological support are therapist-led and self-administered.

In some embodiments, two psychological support sessions, such as two integration sessions are provided subsequent to administration of a first dose and/or a subsequent dose. In some embodiments, the two integration sessions are provided within two weeks after administration. In some embodiments, the two integration sessions are provided within one week after administration. In some embodiments, the first of two integration sessions is provided at or about one day after the psilocybin administration session. In some embodiments the second of two integration sessions is provided about a week after the administration. In some embodiments the second of two integration sessions is provided between at or about 7 days and at or about 14 days after the administration. In some embodiments the second of two integration sessions is provided about 7 days after the administration. In some embodiments the second of two integration sessions is provided 14 days after the administration. In some embodiments the second of two integration sessions is provided about two weeks after the administration.

In some embodiments, the one or more post-administration sessions, or one or more integration sessions, comprises eliciting a complete narrative of the subject's experience during the administration session. In some embodiments, the post-administration sessions, or integration sessions, comprises focused Acceptance and Commitment Therapy (ACT)-based clinical activity related to contacting the present moment, defusion, or acceptance based on clinical formulation. In some embodiments, the post-administration sessions, or integration sessions, comprises reviewing and reflecting upon the subject's psychedelic experience, including any emotional, mental, or lifestyle changes that followed the dosing session.

### 4. Set and Setting

In some aspects, the safety of subjects that are administered a dose of psilocybin is enhanced by testing psilocybin within a “set and setting” protocol, in view of the psychoactive nature of psilocybin (Lyons & Carhart-Harris, J Psychopharmacology 2018; 32(7):811-819), in some cases due to the psychoactive nature of psilocybin. In some aspects, “set” relates to one or more of the emotional, cognitive, behavioral state, mindset, and expectations of subjects prior to, such as immediately prior, to administration of the psychedelic, such as psilocybin. In some aspects, “set” is addressed by the therapist in the pre-dosing psychotherapy sessions. In some aspects, “setting” relates to the physical environment in which administration of psilocybin occurs. In some aspects, by addressing the set and setting of the experience, the risk of a subject reporting a distressing event or injuring themselves can be reduced. In some embodiments, this approach comprises three components: 1) psychological support prior to administration of the psychedelic, such as psilocybin, e.g., preparation, 2) administration, and 3) post-administration psychological support to integrate the classic hallucinogen experience, e.g., integration.

In some embodiments, prior to administration, subjects undergo pre-exposure preparation sessions comprising rapport building with the therapists who would be present during administration. In some embodiments, prior to administration, subjects undergo pre-exposure preparation sessions comprising identifying personal themes and struggles that might be especially likely to impact the session experience. In some embodiments, the administration itself is conducted by one therapist. In some embodiments, the administration itself is conducted by two therapists, such as a male and a female therapist, who are present throughout the session. In some embodiments, sessions are typically conducted in a room designed to be quiet, comfortable, and aesthetically pleasing. In some embodiments, subjects are encouraged to wear eyeshades and listen to a program of music through headphones during the administration to aid them in focusing their attention inward.

In some embodiments, a subject is supervised until an observer judges that the effects of the psychedelic, such as psilocybin, have completely subsided. In some embodiments, the subject can be discharged when meeting the discharge criteria. In some embodiments, one criterion must be met for a subject to be discharged. In some embodiments, more than one criterion must be met for a subject to be discharged. In some embodiments, all criteria must be met for a subject to be discharged. In some embodiments, discharge criteria comprise one or more of whether a responsible friend or family member is available to accompany the subject home, whether the subject's blood pressure and heart rate have returned to pre-drug levels or normal levels, whether the subject is deemed by supervisory personnel to be free of any acute drug effects, whether the subject believes they have returned to their psychological baseline, whether the observer(s) judge that it is safe to discharge the subject, and whether the subject expresses a readiness to go home.

### D. Identification and Exclusion of Subjects for Treatment

Also provided herein are methods for identifying or selecting an individual or a subject, such as a human subject, for treatment with a psychedelic, such as a psilocybin or a metabolite thereof. In some aspects, the psychedelic, such as a psilocybin or a metabolite thereof, is administered to the identified or selected subject, in accordance with the provided embodiments. In some aspects, the subject for treatment has or is suffering from one or more symptoms of nociplastic pain such as fibromyalgia. In some embodiments, the subject is identified or selected as a candidate or as suitable for treatment with a psychedelic if the subject meets one or more criteria for the one or more assessed parameters as described herein. In some embodiments, the subject is identified or selected as a candidate or as suitable for treatment with a psychedelic if the subject does not have any of the exclusion parameters as described herein. In some embodiments, the subject is excluded from treatment or not treated if the subject has or meets the criteria for one or more of the exclusion parameters as described herein.

In some aspects, the one or more parameters, such as exclusion parameters, are assessed prior to administering the one or more doses of a psychedelic and/or psychotherapy. In some embodiments, the one or more parameters, such as exclusion parameters, are assessed in a screening session, for example, prior to administration of the psychedelic and/or psychotherapy. In some aspects, the subject is identified or selected for treatment with a psychedelic and/or psychotherapy, for example, in accordance with the provided embodiments, if the subject meets one or more criteria for the one or more assessed parameters, such as exclusion parameters, In some aspects, the subject is identified or selected for treatment with a psychedelic and/or psychotherapy, for example, in accordance with the provided embodiments, if the subject does not have any of the exclusion parameters, such as exclusion parameters, as described herein.

In some embodiments, exemplary exclusion parameters assessed, for example to identify subjects for treatment, include one or more of: use of prior or concomitant medications, hypertension and/or tachycardia, incidence of epilepsy, incidence of schizophrenia spectrum or other psychotic disorders, a moderate or severe alcohol or drug use disorder, and prior adverse effects from the psychedelic. In some embodiments, the subject is identified for treatment with the psychedelic if the subject meets the criteria for all of the exclusion parameters of: use of prior or concomitant medications, hypertension and/or tachycardia, incidence of epilepsy, incidence of schizophrenia spectrum or other psychotic disorders, a moderate or severe alcohol or drug use disorder, and prior adverse effects from the psychedelic.

In some embodiments, exemplary assessed parameters include one or more of: demographic and baseline characteristics; medical history; fibromyalgia survey score (e.g., 2016 FM Survey Criteria); prior/concomitant medications; Structured Clinical Interview for Diagnostic and Statistical Manual for Mental Disorders 5th Edition (SCID-5) and SCID-5 for Personality Disorders (SCID-5-PD); 12-lead electrocardiogram (ECG); pregnancy test for women of childbearing potential (WOCBP); urine drug and breath alcohol tests (breathalyzer); blood chemistry and hematology tests; hypertension and/or tachycardia; Patient Health Questionnaire depression scale-8 (PHQ-8); and Columbia Suicide Severity Rating Scale (C-SSRS). In some embodiments, exemplary assessed parameters include any indicators that are described herein, for example, for monitoring of treatment, monitoring of adverse effects and/or measuring the effect, such as those indicators described in Sections II and III herein. In some embodiments, one or more of the following parameters are assessed: a fibromyalgia survey score; blood chemistry and hematology tests; hypertension and/or tachycardia; and use of prior or concomitant medications.

In some aspects, the subject is identified or selected for treatment, if the subject has FM, has been diagnosed with FM, is suffering from or has suffered from one or more symptoms of FM, has or has had one or more FM symptoms for a prolonged period, such as at least a year or longer, and/or meets a fibromyalgia survey score criteria, such as a 2016 fibromyalgia (FM) survey criteria (see, e.g., Wolfe et al., Semin Arthritis Rheum 2016; 46(3):319-29). In some aspects, the subject is identified or selected for treatment, if the subject has had a diagnosis of FM for 3 months or longer, has had one or more FM symptoms for at least a year. In some aspects, the subject is identified or selected for treatment, if the subject meets the 2016 FM survey criteria. In some embodiments, the subject meets the criteria if the subject exhibits a fibromyalgia survey score of 13 or more, for example, from the 2016 FM survey criteria.

In some aspects, the subject is identified or selected for treatment, if the subject is considered medically stable, for example as determined by screening for medical problems via a personal interview, a medical questionnaire, and/or an electrocardiogram (ECG), for example, within about 1 month of beginning one or more steps of the treatment regimen. In some embodiments, exemplary methods of assessing medical stability and/or ECG are described. In some embodiments, the methods or uses involve an electrocardiogram (ECG). In some embodiments, the ECG is completed at various time points during the treatment regimen, for example, at screening.

In some aspects, the subject is identified or selected for treatment, if the subject refrains from using any psychoactive drugs, including alcoholic beverages and nicotine, within about 24 hours before and after each the administration of the one or more doses of the psychedelic.

In some aspects, the subject is identified or selected for treatment, if the subject is not undergoing concurrent psychotherapy. In some aspects, the subject is identified or selected for treatment, even with concurrent psychotherapy, if the type and frequency of the therapy has been stable for at least about 2 months prior to the administration or assessment and is expected to remain stable during the treatment regimen.

In some aspects, the subject is identified or selected for treatment, if the subject is a non-smoker (tobacco). In some aspects, the subject is identified or selected for treatment, if the subject does not have a prior adverse effects from the psychedelic.

In some aspects, the subject is identified or selected for treatment, if the subject does not take sildenafil (Viagra®), tadalafil, or similar medications within about 72 hours before and after each the administration of the one or more doses of the psychedelic.

In some aspects, the subject is excluded for treatment or is not identified for treatment, or is not administered the one or more doses of the psychedelic, if the subject has certain co-morbid psychiatric disorders, for example, psychotic disorders, bipolar I or II disorder, for example, has a current or past history of meeting Diagnostic and Statistical Manual of Mental Disorders, 5th edition (DSM-5) criteria for schizophrenia spectrum or other psychotic disorders (except substance/medication-induced or due to another medical condition), or bipolar I or II disorder measured via SCID-5 and SCID-5-PD, or substance use disorder, for example, a current or past history (for example, within 1 year of the time of assessment) of meeting DSM-5 criteria for a moderate or severe alcohol, tobacco, or other drug use disorder (excluding caffeine) measured via relevant questions from the SCID-5, or a history of a medically significant suicide attempt, or has a psychiatric condition judged to be incompatible with establishment of rapport or safe exposure to the psychedelic. In some aspects, suicide risk can be defined by suicidal ideation as endorsed on items 4 or 5 on the C-SSRS within the year prior to the assessment, suicidal behaviors within the year prior to the assessment, or clinical assessment of significant suicidal risk during subject interviews. In some aspects, the subject excluded for treatment, or is not identified or selected for treatment, if the subject has severe depression as measured by PHQ-8. In some embodiments, exemplary methods of assessing such psychiatric disorders or conditions are described.

In some embodiments, the parameter assessed include a structured clinical interview for diagnostic and statistical manual for mental disorders 5^{th} edition (SCID-5) and/or a SCID-5 for personality disorders (SCID-5-PD). In some embodiments, the SCID-5 and/or SCID-5-PD is completed at various time points before and/or during the treatment regimen, for example, at screening.

In some embodiments, the parameter assessed include a patient health questionnaire depression scale-8 (PHQ-8) evaluation. In some embodiments, the PHQ-8 is completed at various time points before and/or during the treatment regimen, for example, at screening.

In some embodiments, the parameter assessed include a Columbia suicide severity rating scale (C-SSRS) evaluation. In some embodiments, the clinical C-SSRS evaluation is completed at various time points during an exemplary treatment regimen, for example, at one or more of: all or nearly all visits and/or sessions.

In some aspects, the subject is excluded for treatment, or is not identified or selected for treatment, if the subject has a confirmed first- or second-degree relative with schizophrenia spectrum or other psychotic disorders (except substance/medication-induced or due to another medical condition), or bipolar I or II disorder.

In some aspects, the parameter is a moderate or severe alcohol or drug use disorder, and the subject is excluded from treatment t if the subject has a moderate or severe alcohol or drug use disorder that meets the DSM-5 criteria.

In some aspects, the subject is excluded for treatment or is not identified for treatment, or is not administered the one or more doses of the psychedelic, if the subject has had, for example, within the year prior to the time of assessment, a cardiovascular condition such as coronary artery disease, stroke, angina, uncontrolled hypertension, a clinically significant ECG abnormality (e.g., atrial fibrillation), prolonged QTc interval (i.e., QTc>450 msec), artificial heart valve, or transient ischemic attack. In some embodiments, exemplary methods of assessing such cardiovascular conditions or abnormalities are described.

In some aspects, the subject is excluded for treatment or is not identified for treatment, or is not administered the one or more doses of the psychedelic, if the subject has epilepsy, such as with a history of seizures. In some aspects, the subject is excluded for treatment or is not identified for treatment, or is not administered the one or more doses of the psychedelic, if the subject has epilepsy. In some embodiments, the subject is identified for treatment if the subject does not have epilepsy.

In some aspects, the subject is excluded for treatment or is not identified for treatment, or is not administered the one or more doses of the psychedelic, if the subject has insulin-dependent diabetes. In some aspects, the subject is excluded for treatment or is not identified for treatment, or is not administered the one or more doses of the psychedelic, if the subject is taking an oral hypoglycemic agent and has a history of hypoglycemia. In some aspects, the subject is excluded for treatment or is not identified for treatment, or is not administered the one or more doses of the psychedelic, if the subject has active auto-immune diseases, such as lupus or rheumatoid arthritis.

In some aspects, the subject is excluded for treatment or is not identified for treatment, or is not administered the one or more doses of the psychedelic, if the subject has uncontrolled hypertension, for example, a systolic blood pressure of greater than at or about 139 mm Hg, a diastolic blood pressure of greater than at or about 89 mm Hg, for example, when measured as an average of three readings. In some aspects, if the subject exhibits a systolic blood pressure of greater than at or about 200 mm Hg, a diastolic blood pressure of greater than at or about 110 mm Hg for more than 15 minutes and/or in an average of 4 consecutive readings, the treatment regimen is immediately discontinued, for example, when measured as an average of three readings.

In some aspects, the subject is excluded for treatment or is not identified for treatment, or is not administered the one or more doses of the psychedelic, if the subject has tachycardia, for example, a heart rate of greater than at or about 90 beats per minute (bpm), for example, with repeated measures.

In some aspects, the subject is excluded for treatment or is not identified for treatment, or is not administered the one or more doses of the psychedelic, if the subject has one or more clinically significant lab abnormalities per clinical laboratory tests, such as a complete blood count, and metabolic panel. In some aspects, abnormalities in metabolic panel can include elevated liver enzymes. In some aspects, the metabolic panel can include measurements of one or more of Na⁺, K⁺, Cl⁻, HCO₃⁻, Ca⁺⁺, Mg⁺⁺, P, BUN, creatinine, glucose, total bilirubin, albumin, ALT, AST, GGT, CK, LDH, and alkaline phosphatase. In some embodiments, hematology laboratory tests include one or more of complete blood count (CBC), CBC with white cell differential and platelet count, white blood cell count (WBC), red blood cell count (RBC), platelet count, and hematocrit red blood cell volume (HCT).

In some aspects, the subject is excluded for treatment or is not identified for treatment, or is not administered the one or more doses of the psychedelic, if the subject is concomitantly taking or using particular medications or therapies, or has taken or used particular medications (for example, one of a list of prohibited medications) or therapies. In some aspects, the subject is excluded for treatment or is not identified for treatment, or is not administered the one or more doses of the psychedelic, if the subject is taking serotonin-acting dietary supplements (e.g., 5-hydroxy-tryptophan or St. John's wort), antihypertensive medications, UDP glucuronosyltransferases (UGT)1A9 or 1A10 inhibitors (e.g., regorafenib, rimampicin, phenytoin, eltrombopag, mefenamic acid, diflunisal, niflumic acid, sorafenib, isavuconazole, deferasiroxor, ginseng), aldehyde or alcohol dehydrogenase inhibitor (e.g., disulfiram), drugs of abuse, psychoactive prescription medications (e.g., opioids, tramadol, benzodiazepines) on a regular basis (i.e., more than twice a week), concurrently taking an antidepressant, has taken any antidepressant medication for at least about 2 prior weeks (or at least 4 prior weeks for fluoxetine), concurrently taking medications having a primary centrally-acting serotonergic effect, including monoamine oxidase inhibitors (MAOIs), on a regular basis (e.g., daily), use of psychoactive drugs, including prescription and recreational drugs other than as part of the treatment regimen, any use of hallucinogens in the prior 6 months or has had a total lifetime hallucinogen use of 10 or more times, above 0.02% blood alcohol content on breath alcohol testing and/or positive for cocaine, methamphetamine, or opioids on urine drug testing. In some aspects, prior or concomitant use of compounds such as primary centrally-acting serotonergic agents, MAOIs, aldehyde dehydrogenase inhibitors, UGT1A9 and UGT1A10 inhibitors, alcohol or tobacco, can influence the effect of the psychedelic, such as psilocybin, as they can affect the metabolism and/or activity of the psychedelic.

In some embodiments, the subject is identified or selected for treatment if the subject is not using any of the following prior or concomitant medications: regular use of psychoactive prescription medication, opioids, tramadol, or benzodiazepines; concomitant use of antidepressants; regular use of medications having a primary centrally-acting serotonergic effect or monoamine oxidase inhibitors (MAOIs); use of hallucinogens in the past 6 months; concomitant use of sildenafil or tadalafil; concomitant use of alcohol with greater than 0.02% blood alcohol content; antihypertensive medications; UGT1A9 inhibitors, 1A10 inhibitors, regorafenib, rimampicin, phenytoin, eltrombopag, mefenamic acid, diflunisal, niflumic acid, sorafenib, isavuconazole, deferasiroxor, ginseng; aldehyde dehydrogenase inhibitors, alcohol dehydrogenase inhibitors, or disulfiram; amphetamines, buprenorphine, benzodiazepines, cocaine, methamphetamines, Ecstasy (MDMA), morphine, methadone, oxycodone, marijuana, ethyl glucuronide, fentanyl, tramadol, synthetic cannabinoids (K2) regular use of psychoactive prescription medication, opioids, tramadol, or benzodiazepines; concomitant use of antidepressants; regular use of medications having a primary centrally-acting serotonergic effect, monoamine oxidase inhibitors (MAOIs), or selective serotonin reuptake inhibitors (SSRIs), or use of serotonin-acting dietary supplements, 5-hydroxy-tryptophan, or St. John's wort, Accordingly, in some aspects, subjects that have had prior or concomitant use of the compounds can be excluded for treatment.

In some aspects, the subject is excluded for treatment or is not identified for treatment, or is not administered the one or more doses of the psychedelic, if the subject does not refrain from using antidepressant medication. In some cases, if a subject initiates a medication taper with consent and support of the physician, the subject can receive administration of the psychedelic and/or psychotherapy. In some aspects, if the subject has intermittently taken agents having a primary centrally-acting serotonergic effect or MAOIs, the subject can receive administration of the psychedelic and/or psychotherapy after at least 5 half-lives of the agent have elapsed after the last administration of the agent. In some aspects, the administration of the psychedelic and/or psychotherapy can be discontinued if the subject takes nonprescription medication, nutritional supplement, or herbal supplement except when approved by a physician.

In some aspects, the subject is an adult, such as an adult between the ages of 25 and 64. In some aspects, the subject has consented to the treatment regimen.

### E. Exemplary Treatment Regimen

An exemplary treatment regimen, in accordance with the provided embodiments, is described. In an exemplary treatment regimen, the clinical benefit of psychedelics, such as oral psilocybin, in conjunction with psychotherapy to treat chronic pain symptoms in patients with FM. In some aspects, the treatment regimen includes baseline deep phenotyping, preparation, psychedelic administration, integration, and EOT deep phenotyping. Individuals, such as human subjects who are candidates for the treatment regimen, undergo an initial pain phenotyping assessment and complete a daily electronic pain diary until the end of active intervention, psychedelic-psychotherapy intervention, the structure of which has been used in previous psychedelic clinical trials begins. During the two psychedelic administration sessions, individuals receive psychedelic in a comfortable setting under the guidance of two therapists. Individuals undergo an in-person visit the day after each psychedelic administration visit to ensure their safety and comfort. Psychotherapy with the same two therapists is held, between and after psychedelic sessions with a focus on integration, helping individuals gain insight and understanding from the experience. After the final dosing session, individuals undergo 3 weekly integration sessions, and then complete a post-treatment deep phenotyping session. Individuals complete subject-reported outcome questionnaires at or about 84 and at or about 168 days after the final psychedelic dose to examine persistence of changes.

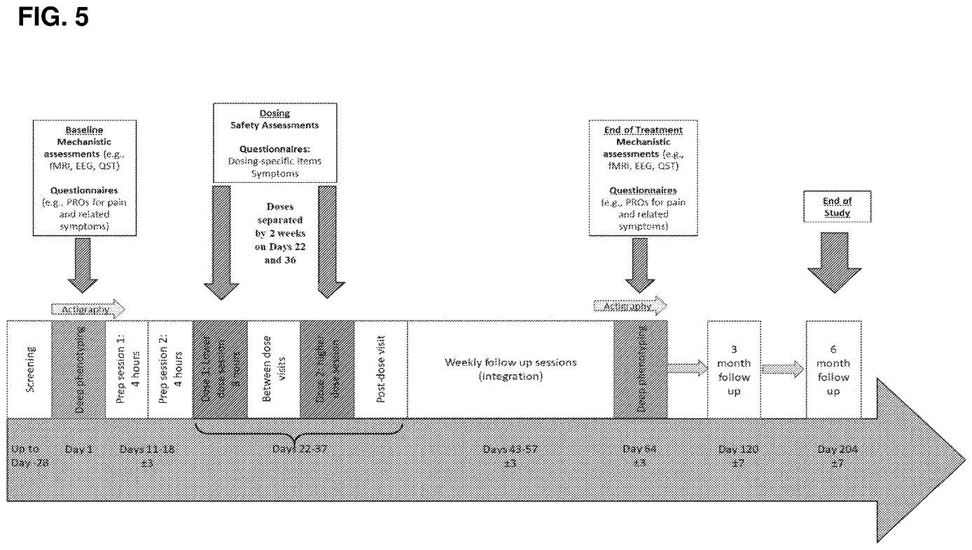

Individuals with FM and with no exclusionary co-morbid psychiatric disorders are treated. An exemplary treatment regimen comprises approximately a 28-day screening period (approximately Days −28 to −1), approximately 3 weeks for baseline and preparation (approximately Days 1 to 18), a 2-dose administration period (approximately Days 22 to 37), integration (approximately Days 43 to 57), the End of Therapy (EOT) visit (approximately Day 64; with last aggregate worst pain score for the 7 days immediately prior to EOT visit assessed for change from baseline) and 1-, 3- and/or 6-month follow-up visits (approximately Days 120 and 204). In an exemplary treatment regimen, the total duration can be approximately 8 months, including screening, preparation, administration, integration, EOT, and follow-up. An exemplary treatment and assessment schedule in accordance with the provided embodiments is depicted in FIG. 5.

### II. Monitoring of Treatment: Subject-Reported Outcomes, Observer-Rated Outcomes, and Clinical Indicators

In some embodiments, one or more indicators are measured, evaluated or assessed to monitor the effect of the treatment. In some aspects, the one or more indicators are associated with the one or more symptoms of fibromyalgia, related to the subject, and/or associated with treatment. In some aspects, the one or more indicators include indicators that are associated with or relate to subject-reported outcomes, observer-rated outcomes, and/or clinical indicators. In some embodiments, the evaluation of the individual receiving treatment comprises subject-reported outcomes, quantitative clinical indicators, and combinations thereof.

In some aspects, the one or more indicators are measured, evaluated or assessed before, during or after the administration of the one or more doses of the psychedelic, such as psilocybin or a metabolite thereof, such as psilocin, and/or the psychotherapy. In some aspects, an individual receiving treatment according to the provided embodiments is subject to one or more evaluation prior to, during, and after treatment. In some aspects, the methods or uses involve assessing in the subject one or more indicators prior to and/or after administration of the one or more doses of the psychedelic. In some aspects, the one or more indicators are assessed prior to the administration as a baseline measurement, and after the administration as an outcome measurement. In some aspects, the changes between the baseline measurement and the outcome measurement for the one or more measured or assessed indicators, are determined. In some aspects, such changes, e.g., changes from baseline, are associated with, or indicate, with a therapeutic outcome of treatment, such as an improvement or amelioration of the one or more symptoms, an effective therapy or an effective treatment of the disease or condition, such as FM or one or more symptoms thereof.

In some aspects, an individual receiving treatment in accordance with the provided embodiments is subject to one or more evaluation prior to, during, and after treatment. In some embodiments, the one or more evaluation of the individual receiving treatment comprises one or more subject-reported evaluation, one or more observer-rated evaluation, one or more quantified clinical indicator, and combinations thereof. In some embodiments, the one or more evaluation of the individual receiving treatment comprises evaluation of primary and/or secondary endpoints.

### A. Subject-Reported and Observer-Rated Indicators

In some embodiments, the evaluation of the individual receiving treatment includes one or more subject-reported or observer-rated evaluation. In some embodiments, the one or more indicators include those related to one or more symptoms of fibromyalgia or those related to diagnosis of fibromyalgia. In some embodiments, the evaluation of the individual receiving treatment includes a qualitative written assessment of treatment experiences, opinions, and insights.

In some aspects, exemplary indicators include an assessment of pain and pain-related symptoms, a sleep assessment, a response to treatment assessment, a psychological evaluation, a psychedelic experience assessment, and/or a clinical activity assessment. In some aspects, one or more indicators measured, evaluated or assessed in accordance with any of the provided embodiments, such as before, during and/or after the administration of the one or more doses of the psychedelic, such as psilocybin, and/or the psychotherapy include, a fibromyalgia survey score, a pain interference assessment, a sleep assessment, a chronic pain acceptance assessment, or a clinical indicator assessment. In some aspects, one or more indicators include observer-rated and subject-reported parameters, for example, related to one or more symptoms or behaviors associated with FM, treatment outcomes and/or the experience related to administration of the psychedelic, such as the psychedelic experience of the subject. In some aspects, one or more of the observer-rated and subject-reported parameters are indicative of treatment effects of the provided embodiments, e.g., resulting in improvement of one or more symptoms associated with FM, in accordance with the provided methods and uses.

In some embodiments, the evaluation comprises one or more assessments selected from among: an aggregate pain intensity numerical rating scale (PI-NRS) assessment, a PROMIS pain interference assessment, a chronic pain acceptance questionnaire (CPAQ-8) assessment, a 2016 fibromyalgia survey questionnaire (FSQ) assessment, a coping strategies questionnaire (CSQ) assessment, a PainDETECT assessment, a PROMIS sleep disturbance 8b assessment, a PROMIS sleep-related impairment 4a assessment, a patient global impression of change (PGI-C) scale assessment, a positive and negative affect score (PANAS) assessment, a PROMIS-29+2 Profile v2.1 multi-domain assessment, a patient acceptable symptom state (PASS) questionnaire, a mystical experience questionnaire (MEQ30), a challenging experiences questionnaire (CEQ), a psychological insight questionnaire (PIQ), and/or a monitor rating scale (MRS) assessment.

In some embodiments, the one or more indicators are evaluated or measured at various time points during an exemplary treatment regimen, for example, at one or more of: baseline deep profiling, first dose, second dose, end of therapy deep profiling, 1 month follow-up, 3 month follow-up, and 6 month follow-up.

### 1. Pain Assessment and Characterization

In some embodiments, the one or more indicators include those related to one or more symptoms of fibromyalgia (FM), such as pain, or those related to the diagnosis of FM. Exemplary indicators related to one or more symptoms of FM or pain include a fibromyalgia survey score, such as a 2016 fibromyalgia survey questionnaire (FSQ) assessment. In some embodiments, exemplary indicators related to one or more symptoms of FM, such as pain, include one or more of: a fibromyalgia survey score, such as a 2016 fibromyalgia survey questionnaire (FSQ) assessment, a pain intensity numerical rating scale (PI-NRS) assessment, such as an 11-item PI-NRS scale; a PROMIS pain interference assessment; a PainDETECT Questionnaire (PD-Q) assessment; a chronic pain acceptance assessment, such as measured by chronic pain acceptance questionnaire (CPAQ-8) assessment; a pain interference assessment, such as measured by 4-item PROMIS pain interference scale; a complex medical symptom inventory (CMSI), and a Brief Pain Inventory, such as measured by an interference sub-scale (BPI-IS).

a. FM Survey Score

In some embodiments, the one or more indicators include a FM survey score. Exemplary FM survey score includes a 2016 FM survey questionnaire (FSQ) assessment. The 2016 FM survey criteria, also referred to as the FSQ, is a revised diagnostic tool that combines aspects of other established pain evaluations to provide reliable FM diagnoses. The FSQ includes the measure of widespread pain index (WPI) at 19 sites, which is derived from the Michigan Body Map (Brummett et al., Pain 2016; 157(6):1205-12), wherein each painful site is scored as 1 point and the painful sites are summed. The FSQ also includes a symptom severity scale (SSS) assessment (e.g., fatigue, cognitive issues, headache, poor mood) (Neville et al., Clin J Pain 2018 34(10):909-17; Brummett et al., Arthritis Rheumatol 2015; 67(5):1386-94; Janda et al., Anesthesiology 2015; 122(5):1103-11; Brummett et al., Anesthesiology 2013; 119(6):1434-43), wherein 3 items are scored 0-3 with 3 more items scored 0 or 1, and summed (0-12). Together, the WPI and SSS scores are used to form a continuous proxy index of nociplastic pain characteristics (range 0 to 31) (Wolfe et al., Semin Arthritis Rheum 2016; 46(3):319-29; Wolfe et al., J Rheumatol 2011; 38(6):1113-22; Wolfe F, Arthritis Rheum 2009; 61(6):715-16). Based on the individual WPI and SSS scores, as well as the total score, an accurate FM diagnosis is possible.

In some embodiments, the FSQ is repeated one or more times, for example, before, during and/or after administration of the psychedelic, such as psilocybin, and/or psychedelic support, such as an integration session. In some aspects, the embodiments provided herein include assessment of a change from baseline in FSQ. In some aspects, the FSQ evaluation is completed at one or more of the time points following administration of one or more doses of the psychedelic, such as between at or about 2 weeks and 20 weeks after administration of one or more doses of the psychedelic, such as at or about 2 weeks, 4 weeks, 6 weeks, 8 weeks, 10 weeks, 12 weeks, 14 weeks and/or 16 weeks after the administration. In some embodiments, the FSQ score for an individual receiving treatment decreases during and/or after treatment completion, wherein the FSQ score decreases 1 or more points, 2 or more points, 3 or more points, 4 or more points, 5 or more points, 6 or more points, 7 or more points, 8 or more points, 9 or more points, 10 or more points, 11 or more points or 12 or more points.

b. Pain Intensity Numeric Rating Scale (PI-NRS) Score

In some embodiments, the one or more indicators include a pain intensity assessment, such as the pain intensity numeric rating scale (PI-NRS), including an aggregate PI-NRS. In some aspects, the PI-NRS (also called numerical pain rating scale or NPRS) is an self-reported 11-point rating scale used to assess the severity of pain experienced by an individual (Farrar et al., Pain 2001; 94(2):149-58). An individual rates their pain symptoms using a numerical scaled from 0-10, wherein, for example, 0 represents “no pain” and 10 represents “pain as bad as it could be” or “the worst pain imaginable.” Individuals are often instructed to complete this rating daily to help monitor the change in pain intensity over time, for example, during treatment. In some aspects, scores at baseline (Days 1 to 7) are aggregated and compared to the terminal 7 day aggregate (Day 57 to 63) immediately prior to the end of therapy (Day 64) to determine a change in PI-NRS score. A reduction of approximately 2 points on the PI-NRS, or roughly a 30% decrease, corresponds with a clinically important difference (Farrar et al., Pain 2001; 94(2):149-58).

In some embodiments, the methods or uses involve an assessment of the maximum change from baseline in aggregate worst pain after treatment completion, wherein the aggregate worst pain is measured by a pain intensity numeric rating scale (PI-NRS) assessment. In some embodiments, the assessment includes one or more aggregate worst pain evaluation before, during, and/or after treatment completion. In some embodiments, the assessment concludes immediately prior to the end of therapy, wherein terminal aggregate pain is compared to baseline aggregate pain to determine the maximum change from baseline in aggregate worst pain after treatment completion.

In some embodiments, the PI-NRS is utilized to evaluate an effect of the psychedelic for improving one or more symptoms of FM. In some embodiments, the PI-NRS is repeated one or more times. In some embodiments, the individual receiving treatment completes 4 or more PI-NRS evaluations a week, 5 or more PI-NRS evaluations a week, 6 or more PI-NRS evaluations a week, of 7 or more PI-NRS evaluations a week, wherein this routine in continued until the therapy is completed. In some embodiments, two or more PI-NRS evaluation scores from adjacent or nearby days are aggregated together. In some embodiments, a change from baseline in the aggregated PI-NRS score is utilized to evaluate an effect of the psychedelic for improving one or more symptoms of FM. In some embodiments, the PI-NRS score for an individual receiving treatment decreases during and/or after treatment completion, wherein the PI-NRS score decreases 1 or more points, 2 or more points, 3 or more points, 4 or more points, 5 or more points, or 6 or more points. In some embodiments, the aggregated PI-NRS score for an individual receiving treatment decreases during and/or after treatment completion, wherein the PI-NRS score decreases 1 or more points, 2 or more points, 3 or more points, 4 or more points, 5 or more points, or 6 or more points compared to baseline.

c. Pain Interference—PROMIS

In some embodiments, the one or more indicators include a pain interference assessment, such as a PROMIS pain interference assessment, for example, the PROMIS-29+2 Profile. In some aspects, pain interference is the degree to which pain affects important aspects of an individual's daily life, such as social interactions, cognitive tasks, and physical activities (Adams et al., J Pain 2018; 19(9):1074-81). Pain interference is assessed using the patient-reported outcomes measurement information system (PROMIS) pain interference (PI) scale from the PROMIS-29+2 Profile v2.1 (PROPr) (see, e.g., HealthMeasures.net). In some aspects, the PI evaluation can contain 4 items and individuals respond to each item using a 5-point Likert scale, before converting the raw score to a standardized T-score using conversion tables (see, e.g., nihpromis.org), wherein higher scores correspond to more significant pain interference.

In some embodiments, the PROMIS-29+2 PI is utilized to evaluate an effect of the psychedelic for improving one or more symptoms of FM. In some embodiments, the PROMIS-29+2 PI evaluation is repeated for the individual receiving treatment at various time points during an exemplary treatment regimen, for example, at one or more of: baseline deep profiling, first dose, second dose, end of therapy deep profiling, 1-month follow-up, 3-month follow-up and/or 6-month follow-up. In some embodiments, the PROMIS-29+2 PI score for an individual receiving treatment decreases during and/or after treatment completion. In some cases, the PROMIS-29+2 PI score decreases 1 or more points, 2 or more points, 3 or more points, 4 or more points, 5 or more points, or 10 or more points compared to baseline.

d. Chronic Pain Acceptance Questionnaire (CPAQ-8)

In some embodiments, the one or more indicators include a pain avoidance or acceptance assessment, such as a chronic pain acceptance questionnaire 8 (CPAQ-8) assessment. In some aspects, individuals living with chronic severe pain often exhibit pain avoidance, wherein the individual seeks to control or avoid their pain, despite these behaviors being maladaptive and/or counterproductive (Volders et al., Behav Res Ther 2015; 64:31-7). In some aspects, the CPAQ-8 is a validated evaluation for assessing this behavior by focusing on activity engagement and pain willingness (Fish et al., Pain 2010; 149(3):435-43). In some cases, items 1, 3, 5, and 6 address activity engagement and items 2, 4, 7, and 8 address pain willingness. Individuals rate 8 items on a 7-point scale from 0 to 6, wherein 0 is “never true,” 1 is “very rarely true,” 2 is “seldom true,” 3 is “sometimes true,” 4 is “often true,” 5 is “almost always true,” and 6 is “always true.” All responses are summed (0 to 48 range) and evaluated, wherein a higher total score corresponds to higher levels of chronic pain acceptance.

In some embodiments, the CPAQ-8 is utilized to evaluate an effect of the psychedelic for improving one or more symptoms of FM. In some embodiments, the CPAQ-8 evaluation is repeated for the individual receiving treatment at various time points during an exemplary treatment regimen, for example, at one or more of: baseline deep profiling, end of therapy deep profiling, 1-month follow-up, 3-month follow-up and/or 6-month follow-up. In some embodiments, the total CPAQ-8 score for an individual receiving treatment increases during and/or after treatment completion. In some aspects, the CPAQ-8 score increase 1 or more points, 2 or more points, 3 or more points, 4 or more points, 5 or more points, or 10 or more points. In some embodiments, the activity engagement CPAQ-8 score for an individual receiving treatment increases during and/or after treatment completion. In some aspects, the CPAQ-8 score increase 1 or more points, 2 or more points, 3 or more points, 4 or more points, 5 or more points, or 10 or more points. In some embodiments, the pain willingness CPAQ-8 score for an individual receiving treatment decreases during and/or after treatment completion. In some aspects, the CPAQ-8 score increase 1 or more points, 2 or more points, 3 or more points, 4 or more points, 5 or more points, or 10 or more points compared to baseline.

e. Coping Strategies Questionnaire (CSQ) for Pain Catastrophizing

In some embodiments, the one or more indicators include a pain catastrophizing assessment, such as a coping strategies questionnaire (CSQ) assessment. In some aspects, pain catastrophizing is characterized by the constant and uncontrollable fixation on a painful encounter, before, during, or after the painful encounter, frequent magnification of the threat of pain, and generally feeling helpless or paralyzed in the context of pain (Quartana et al., 2009; 9(5):745-58). The coping strategies questionnaire (CSQ) is a self-reported 6-item evaluation that assesses pain catastrophizing, addressing pain-related helplessness and pessimism (Rosenstiel and Keefe Pain 1983; 17(1):33-44), which is strongly associated with the acute pain to chronic pain transition, as well as poorer outcomes (Kratz et al., J Pain 2015; 16(6):527-36). Individuals respond to each item using a 7-point scale from 0 to 6, wherein 0 is “never do,” 3 is “sometimes do that,” and 6 is “always do that.” Responses are summed and evaluated, with a higher total score corresponding to higher levels of coping and pain catastrophizing.

In some embodiments, the CSQ is utilized to evaluate an effect of the psychedelic for improving one or more symptoms of FM. In some aspects, the indicator assessed includes a modified CSQ, termed Pain Castrophizing Scale (PCS) (see, e.g., Sullivan et al., Psychological assessment 7.4 (1995): 524). In some embodiments, the CSQ evaluation is repeated for the individual receiving treatment at various time points during an exemplary treatment regimen, for example, at one or more of: baseline deep profiling, end of therapy deep profiling, 1-month follow-up, 3-month follow-up and/or 6-month follow-up. In some embodiments, the total CSQ score for an individual receiving treatment decreases during and/or after treatment completion. In some embodiments, the CSQ score decreases 1 or more points, 2 or more points, 3 or more points, 4 or more points, 5 or more points, or 10 or more points compared to baseline.

f. Neuropathic Pain—PainDETECT Questionnaire (PD-Q)

In some embodiments, the one or more indicators include an assessment of neuropathic pain, such as a PainDETECT Questionnaire (PD-Q) assessment. Neuropathic pain is associated with a lesion or disease of the somatosensory system (Bouhassira D, Rev Neurol (Paris) 2019; 175(1-2):16-25) and it contribute to the overall pain experience, along with nociceptive pain (i.e., caused by a tissue injury or bodily damage). In some aspects, the PainDETECT questionnaire (PD-Q) is a self-reported 9-item evaluation used to determine the prevalence of neuropathic pain (Freynhagen et al., Curr Med Res Opin 2006; 22(10):1911-20). Among these 9 items there are seven sensory symptoms items for pain, one temporal item on pain-course pattern, and one spatial item on pain radiation, wherein each item category has a separate scoring system (Cappelleri et al., Clinicoecon Outcome Res 2014; 6:497-504). Individuals respond to sensory symptoms items using a 6-point scale from 0 to 5, wherein 0 is never and 5 is strongly. Individuals respond to the temporal item using a 3-point scale from −1 to 1 and the one spatial item using a 4-point scale from 0 to 2, wherein 0 is no radiation and 2 is radiating pain. Responses are summed and evaluated, with a higher total score corresponding to higher levels of neuropathic pain.

In some embodiments, the PD-Q evaluation is utilized to evaluate an effect of the psychedelic for improving one or more symptoms of FM. In some embodiments, the PD-Q evaluation is repeated for the individual receiving treatment at various time points during an exemplary treatment regimen, for example, at one or more of: baseline deep profiling, end of therapy deep profiling, 1-month follow-up, 3-month follow-up and/or 6-month follow-up. In some embodiments, one or more PD-Q score for an individual receiving treatment decreases during and/or after treatment completion. In some aspects, one or more PD-Q score decreases by 1 or more points, 2 or more points, 3 or more points, 4 or more points, 5 or more points, or 10 or more points compared to baseline.

g. Complex Medical Symptom Inventory (CMSI)

In some embodiments, the one or more indicators include a complex medical symptom inventory (CMSI). The CMSI is a 52-item inventory that measures presence or absence of persistent physical symptoms (lasting at least 3 months) over the past year and during one's lifetime (see, e.g., Geisser et al., J. Pain 9(4, S2):75; Williams et al., Rheum Dis Clin North Am. 2016 May; 42(2): 317-332). In some aspects, CSMI is used to screen for chronic overlapping pain conditions, as other pain conditions commonly co-occur in FM. In some embodiments, the methods or uses involve a CMSI evaluation. In some embodiments, the CMSI is completed at various time points during the treatment regimen, for example, at deep phenotyping.

In some embodiments, the CMSI is utilized to evaluate an effect of the psychedelic for improving one or more symptoms of FM. In some embodiments, the CMSI evaluation is repeated for the individual receiving treatment at various time points during an exemplary treatment regimen, for example, at one or more of: baseline deep profiling, end of therapy deep profiling, 1-month follow-up, 3-month follow-up and/or 6-month follow-up. In some embodiments, the CMSI score for an individual receiving treatment decreases during and/or after treatment completion. In some aspects, the CMSI score is decreased by 1% or more, 2% or more, 3% or more, 4% or more, 5% or more, 10% or more, 15% or more, 20% or more, or at least 25% compared to baseline.

h. Brief Pain Inventory, Interference sub-scale (BPI-IS)

In some embodiments, the one or more indicators include a Brief Pain Inventory (BPI) (also called Wisconsin Brief Pain Questionaire). The BPI is a validated self-report measure of pain that can be assessed on two sub-scales: (1) pain intensity (i.e. worse, least, average, current) measured by a “Pain Severity Score”; and (2) functional interference from pain (i.e. activity, mood, walking ability, normal work, relations with others, sleep, and life enjoyment), measured by a “Pain Interference Scale.” See e.g., Williams and Arnold, Arthritis Care Res, 2011, 63:S86-S97. For both the “severity” and “interference” scales, the scores range from 0 to 10 with higher scores indicating greater pain. In some embodiments, the one or more indicators is based on the interference sub-scale. In some embodiments, the methods or uses involve a BPI evaluation, such as using the interference sub-scale.

In some embodiments, the BPI evaluation is utilized to evaluate an effect of the psychedelic for improving one or more symptoms of FM. In some embodiments, the BPI evaluation is repeated for the individual receiving treatment at various time points during an exemplary treatment regimen, for example, at one or more of: baseline deep profiling, end of therapy deep profiling, 1-month follow-up, 3-month follow-up and/or 6-month follow-up. In some embodiments, one or more BPI score for an individual receiving treatment decreases during and/or after treatment completion. In some aspects, one or more BPI score decreases by 1 or more points, 2 or more points, 3 or more points, 4 or more points, 5 or more points, or 10 or more points compared to baseline.

### 2. Sleep Assessment and Characterization

In some embodiments, the one or more indicators include those related to one or more symptoms of FM, such as disturbance in sleep or assessing sleep patterns. Exemplary indicators related to one or more symptoms of FM such as sleep disturbance, include a sleep impairment assessment, optionally measured by a PROMIS sleep-related impairment 4a assessment; a PROMIS sleep disturbance 8b assessment; and a STOP-Bang assessment. In some embodiments, the one or more indicators include a PROMIS-29+2 Profile, optionally a v2.1 multi-domain assessment.

In some aspects, individuals with FM and pain-related disorders often experience a sleep disturbance, problems sleeping, a decline in physical, and depression. In this context, sleep disturbance and problems impacting the quality of sleep can come in many forms, including but not limited to difficulty falling asleep, insomnia, frequent nightmare awakenings, hyperarousal, sleep apnea, restless leg syndrome, and increased restless sleep, leading to increased daytime sleeping and increased nighttime movement and interruptions (Bigatti et al., Arthritis Rheum 2008; 59(7):961-7). Sleep disturbance, sleep problems and sleep deprivation are likely to exacerbate pain sensitivity, tolerance, intensity, and interference, while also impacting an individual's mood, behavior, activity level, and physical state (Choy E, Nat Rev Rheumatol 2015; 11(9):513-20). In some aspects, sleep-related problems can be closely monitored and evaluated during the treatment of an individual with FM or a pain-related disorder, such as in accordance with the methods and uses provided herein. In some embodiments, a sleep assessment is utilized to evaluate an effect of the psychedelic for improving one or more symptoms of FM. In some embodiments, one or more sleep assessment is utilized to evaluate an effect of the psychedelic for improving one or more symptoms of FM and related pain disorders. In some embodiments, exemplary indicators related to sleep include the PROMIS sleep-related disturbance 8b assessment, the PROMIS sleep-related impairment 4a assessment, or a STOP-Bang assessment. In some embodiments, one or more evaluation is completed at various time points during an exemplary treatment regimen, for example, at one or more of: baseline deep profiling, end of therapy deep profiling, 1-month follow-up, 3-month follow-up and/or 6-month follow-up.

a. PROMIS Sleep Disturbance 8b

In some embodiments, the one or more indicators include a PROMIS sleep disturbance assessment. In some aspects, assessment of sleep disturbance includes assessment of sleep quality, perceived ability to fall and stay asleep, satisfaction of sleep, and depth of sleep (Cella et al., J Clin Epidemiol 2011; 63(11):1179-94). In some aspects, sleep disturbance is measured using the PROMIS Sleep Disturbance Short Form 8b (see, e.g., HealthMeasures.net). In some aspects, this evaluation can contain 4 items and individuals respond to each item using a 5-point Likert scale, before converting the raw score to a standardized T-score using conversion tables (see, e.g., nihpromis.org), wherein higher scores correspond to more significant sleep disturbance.

In some embodiments, the PROMIS Sleep Disturbance 8b is utilized to evaluate an effect of the psychedelic for improving one or more symptoms of FM. In some embodiments, the PROMIS Sleep Disturbance 8b evaluation is repeated for the individual receiving treatment at various time points during an exemplary treatment regimen, for example, at one or more of: baseline deep profiling, end of therapy deep profiling, 1-month follow-up, 3-month follow-up and/or 6-month follow-up. In some embodiments, the total PROMIS Sleep Disturbance 8b score for an individual receiving treatment decreases during and/or after treatment completion. In some aspects, the PROMIS Sleep Disturbance 8b score is decreased by 1% or more, 2% or more, 3% or more, 4% or more, 5% or more, 10% or more, 15% or more, 20% or more, or at least 25% compared to baseline.

b. PROMIS Sleep-related Impairment 4a

In some embodiments, the one or more indicators include a PROMIS sleep impairment assessment. Similar to sleep disturbance, sleep impairment relates to the functional impairments during wakefulness that are associated with one or more sleep problems (Hanish et al., Nurs Res 2017; 66(3):246-51). The PROMIS Sleep-related Impairment 4a evaluation focuses on perceptions of alertness, sleepiness, tiredness, and functional impairment during normal waking hours. In some aspects, this evaluation can contain 4 items and individuals respond to each item using a 5-point Likert scale, before converting the raw score to a standardized T-score using conversion tables (see, e.g., nihpromis.org), wherein higher scores correspond to more significant sleep disturbance.

In some embodiments, the PROMIS Sleep-related Impairment 4a is utilized to evaluate an effect of the psychedelic for improving one or more symptoms of FM. In some embodiments, the PROMIS Sleep-related Impairment 4a evaluation is repeated for the individual receiving treatment at various time points during an exemplary treatment regimen, for example, at one or more of: baseline deep profiling, end of therapy deep profiling, 1-month follow-up, 3-month follow-up and/or 6-month follow-up. In some embodiments, the total PROMIS Sleep-related Impairment 4a score for an individual receiving treatment decreases during and/or after treatment completion. In some aspects, the PROMIS Sleep-related Impairment 4a score decreases by 1% or more, 2% or more, 3% or more, 4% or more, 5% or more, 10% or more, 15% or more, 20% or more, or at least 25% compared to baseline.

c. STOP-Bang

In some embodiments, the one or more indicators include a STOP-Bang assessment. In some aspects, STOP-Bang (Chung et al., Chest 2016; 149(3):631-38; Nagappa et al., PLoS One 2015; 10(12):e0143697) is used to screen for sleep apnea which can interfere with sleep. In some aspects, the STOP-Bang questionnaire consists of 8 items that assess risk of sleep apnea, including snoring, tiredness, whether anyone has observed the person stop breathing in their sleep, high blood pressure, body mass index, age, neck circumference>16 inches, and sex. In some embodiments, the methods or uses involve a STOP-Bang evaluation. In some embodiments, the STOP-Bang is completed at various time points during the treatment regimen, for example, at deep phenotyping.

In some embodiments, the STOP-Bang is utilized to evaluate an effect of the psychedelic for improving one or more symptoms of FM. In some embodiments, the STOP-Bang evaluation is repeated for the individual receiving treatment at various time points during an exemplary treatment regimen, for example, at one or more of: baseline deep profiling, end of therapy deep profiling, 1-month follow-up, 3-month follow-up and/or 6-month follow-up. In some embodiments, the total STOP-Bang evaluation for an individual receiving treatment decreases during and/or after treatment completion. In some aspects, the STOP-Bang evaluation is improved compared to baseline.

### 3. Response to Treatment Assessments and Psychological Evaluations

In some embodiments, the one or more indicators include those related to treatment and psychological status, such one or more subject-reported parameters. In some aspects, to evaluate an individual receiving treatment in accordance with the provided embodiments, the individual is subject to a one or more evaluation to determine the response (e.g., change from baseline) to treatment and psychological status (Davis et al., J Psychopharmacol 2021; 35(4):437-46). In some aspects, different approaches for quantification of treatment outcomes for administration of psychedelics have been employed (Davis et al., J Psychopharmacol 2021; 35(4):437-46; Haijen et al., Front Pharmacol 2018; 9:897). Exemplary approaches often include observer-rated and subject-reported outcomes, administered in the form of a time-sensitive survey.

In some embodiments, exemplary indicators for evaluation to determine the response to treatment and psychological status include one or more of: a PROMIS-29+2 Profile, optionally a v2.1 multi-domain assessment; a positive and negative affect score (PANAS) assessment; a patient global impression of change (PGI-C) scale assessment; and a patient acceptable symptom state (PASS) questionnaire.

In some aspects, exemplary indicators for evaluation to determine the response to treatment and psychological status include one or more of: a Patient Health Questionnaire depression scale-8 (PHQ-8); a Columbia Suicide Severity Rating Scale (C-SSRS); a Clinician Global Impression—Improvement (CGI-I) scale; an Emotional Breakthrough Inventory (EBI); a Acceptance and Action Questionnaire-II (AAQ-II); a Patient Global Impression-Improvement (PGI-I); a Hospital Anxiety and Depression Scale (HADS); a life events checklist (LEC); a Multidimensional Experiential Avoidance Questionnaire (MEAQ); and a Brief Experiential Avoidance Questionnaire (BEAQ).

In some embodiments, one or more evaluation is completed for the individual receiving treatment at various time points during an exemplary treatment regimen, for example, at one or more of: baseline deep profiling and first dose through 6-month follow-up. In some embodiments, an evaluation is completed at 1 month after the dose. In some embodiments, an evaluation is completed at 2 months after the dose. In some embodiments, an evaluation is completed at 3 months after the dose. In some embodiments, an evaluation is completed at 4 months after the dose. In some embodiments, an evaluation is completed at 5 months after the dose.

a. Patient Global Impression of Change (PGI-C)

In some embodiments, the one or more indicators include a patient global impression of change (PGI-C). In some aspects, the PGI-C is a patient reported measure that reflects a patient's belief regarding the effect of treatment (Guy W., Assessment Manual for Psychopharmacology (revised) 1976:217-21). Individuals receiving treatment then rate their current condition on a scale from 1-7, wherein 1 is very much improved since baseline (initiation of treatment), 4 is no change from baseline, and 7 is very much worse from baseline.

In some embodiments, the PGI-C scale is utilized to evaluate an effect of the psychedelic for improving one or more symptoms of FM. In some embodiments, the PGI-C evaluation is completed for the individual receiving treatment at various time points during an exemplary treatment regimen, for example, at one or more times between first dose through 6-month follow-up. In some embodiments, the PGI-C score for an individual receiving treatment decreases during and/or after treatment completion. In some aspects, the PGI-C score decreases 1 or more points, 2 or more points, 3 or more points, 4 or more points, or 5 or more points.

b. Positive and Negative Affect Score (PANAS)

In some embodiments, the one or more indicators include a positive and negative affect schedule (PANAS). Positive and negative affect (e.g., emotions and expressions) are two opposing states that describe contrasting perspectives for viewing life and its associated challenges. The PANAS is a 20-item evaluation that measures positive and negative affect (Watson et al., J Pers Soc Psychol 1988; 54(6):1063-70), including a metric of affect balance, such as greater negativity over positivity (McAllister et al., Stress Health 2015; 31(4):299-305; Toussaint et al., Scand J Pain 2014; 5(3):161-66; Hassett et al., Arthritis Rheum 2008; 59(6):833-40). Items 1, 3, 5, 9, 10, 12, 14, 16, 17, and 19 address positive affect and items 2, 4, 6-8, 11, 13, 15, 18, and 20 address negative affect. Further, the PANAS can be used to evaluate a trait or a state, depending on the time scale used. In some aspects, the state measure is used. Individuals respond to each item using a 5-point Likert scale from 1 to 5, wherein 1 is very slightly or not at all, 3 is moderately, and 5 is extremely. Responses are summed for each subscale separately, with higher scores corresponding to higher levels positive/negative affect (and vice versa).

In some embodiments, the PANAS evaluation is utilized to evaluate an effect of the psychedelic for improving one or more symptoms of FM. In some embodiments, the PANAS evaluation is repeated for the individual receiving treatment at various time points during an exemplary treatment regimen, for example, at one or more of: baseline deep profiling, end of therapy deep profiling, 1-month follow-up, 3-month follow-up and/or 6-month follow-up. In some embodiments, the PANAS positive affect score for an individual receiving treatment increases during and/or after treatment completion. In some aspects, the PANAS positive affect score increases 1 or more points, 2 or more points, 3 or more points, 4 or more points, 5 or more points, or 10 or more points compared to baseline. In some embodiments, the PANAS negative affect score for an individual receiving treatment decreases during and/or after treatment completion. In some aspects, the PANAS negative affect score decreases 1 or more points, 2 or more points, 3 or more points, 4 or more points, 5 or more points, or 10 or more points compared to baseline.

c. Multi-Domain Analysis-PROMIS-29+2 Profile v2.1

In some embodiments, the one or more indicators include a PROMIS-29+2 Profile v2.1 (PROPr) (see, e.g., HealthMeasures.net). In some aspects, the PROPr is described is used to assess multiple domains, including physical function, anxiety, depression, fatigue, sleep disturbance, ability to participate in social roles, pain interference, cognitive function, and pain intensity. Addition of the two cognitive functioning items differentiates this instrument from its parent the PROMIS Profile 29 (Hays et al., Qual Life Res 2018; 27(7):1885-1891). The PROMIS-29+2 is used to calculate a preference score (PROMIS Preference, PROPr) (Dewitt et al., Value Health 2020; 23(3):370-78; Dewitt et al., Med Decis Making 2018; 38(6):683-98; Hanmer et al., PLoS One 2018; 13(7):e0201093), which provides an overall summary of health-related quality of life on a common metric. For each domain, a preference-based scores is calculated ranging from 0 (as bad as dead) to 1 (perfect or ideal health). In some cases, responses are summed for each subscale separately, with higher scores corresponding to higher levels of domain character.

In some embodiments, the PROPr evaluation is utilized to evaluate an effect of the psychedelic for improving one or more symptoms of FM. In some embodiments, the PROPr evaluation is repeated for the individual receiving treatment at various time points during an exemplary treatment regimen, for example, at one or more of: baseline deep profiling, first dose, second dose, end of therapy deep profiling, 1-month follow-up, 3-month follow-up and/or 6-month follow-up. In some embodiments, one or more PROPr domain score for an individual receiving treatment increases during and/or after treatment completion. In some aspects, one or more PROPr domain score increases 1 or more points, 2 or more points, 3 or more points, 4 or more points, 5 or more points, or 10 or more points compared to baseline.

d. Patient Acceptable Symptom State (PASS) Questionnaire

In some embodiments, the one or more indicators include a patient acceptable symptom state (PASS) questionnaire. In some aspects, in addition to the PROPr, one or more items from the PASS questionnaire can be used. Addition of this elements to each domain allows for the assessment and collection of patient belief in regards to the adequacy of domain management (Salaffi et al., Biomed Res Int 2015:930756). Individual receiving treatment respond to the following question: “Considering all the different ways your disease is affecting you, if you were to stay in this state for the next few months, do you consider that your current state is satisfactory?” Individuals respond with using yes or no, and this response is accounted in overall individual status and improvement.

In some embodiments, the PASS evaluation is utilized to evaluate an effect of the psychedelic for improving one or more symptoms of FM. In some embodiments, the PASS evaluation is completed at various time points during an exemplary treatment regimen, for example, at one or more of: baseline deep profiling, first dose, second dose, end of therapy deep profiling, 1-month follow-up, 3-month follow-up and/or 6-month follow-up. In some embodiments, the PASS response for an individual receiving treatment improves compared to baseline.

e. Clinical Global Impression-Improvement Scale (CGI-I)

The clinical global impression-improvement (CGI-I) evaluation is a clinician-rated change in the medical condition of an individual receiving treatment, wherein the individual is compared to baseline conditions, sometimes within the past seven days (Guy W., Assessment Manual for Psychopharmacology (revised) 1976:217-21). Individuals receiving treatment are rated on a scale from 1-7 for baseline conditions, wherein 1 is not ill at all and 7 is among the most extremely ill patients. Individuals receiving treatment are then rated on a scale from 1-7 for current condition, wherein 1 is “very much improved” since baseline (initiation of treatment), 4 is no change from baseline, and 7 is “very much worse” from baseline. In some embodiments, the change from baseline in CGI-I scale is utilized to evaluate the treatment of FM.

In some embodiments, the method provided herein includes a change from baseline in clinical global impression-improvement (CGI-I) evaluation. In some aspects, the CGI-I evaluation is completed at one or more of the time points following administration of one or more doses of the psychedelic, such as at one or more of: baseline deep profiling, first dose, second dose, end of therapy deep profiling, 1-month follow-up, 3-month follow-up and/or 6-month follow-up.

In some embodiments, the CGI-I score for an individual receiving treatment improves during and/or after treatment. In some aspects, the CGI-I score decreases 1 or more points, 2 or more points, 3 or more points, 4 or more points, or 5-7 points. In some aspects, CGI-I related improvement involves decreasing the baseline conditions score and decreasing the current condition score.

f. Hospital Anxiety and Depression Scale (HADS)

The hospital anxiety and depression scale (HADS) evaluation aims to measure psychological distress, symptoms of anxiety and depression using a 14 item questionnaire, wherein seven items are designated for the depression subscale (HADS Depression) and seven items are designated for the anxiety subscale (HADS Anxiety) (Zigmond and Snaith, Acta Psychiatr Scand 1983; 67(6):361-70). The depression branch focuses specifically on anhedonia, or the ability to feel pleasure, while the anxiety branch addresses general symptoms common to many anxiety disorders. As a means of evaluation, each HADS item is scored on a four-option response-scale ranging between 0 and 3. Responses are then summed to obtain a total score for each two subscales, wherein an individual can be assessed as normal 0-7, borderline abnormal (case) 8-10, or abnormal (case) 11-21. Interpretation using a cut-off score of 8 or above for both HADS Anxiety and HADS Depression subscales is generally regarded as best practice.

In some aspects, the embodiments provided herein include assessment of a change from baseline in hospital anxiety and depression scale (HADS) evaluation. In some aspects, the HADS evaluation is completed at one or more of the time points following administration of one or more doses of the psychedelic, such as at one or more of: baseline deep profiling, first dose, second dose, end of therapy deep profiling, 1-month follow-up, 3-month follow-up and/or 6-month follow-up.

In some embodiments, the HADS is utilized to evaluate the treatment of FM. In some embodiments, the HADS score for an individual receiving treatment decreases during and/or after treatment. In some aspects, the HADS score decreases by at least at or about 1 point, 2 points, 3 points, 4 points, 5 points, 6 points, 7 points, or 8 points, or more.

g. Acceptance and Action Questionnaire (AAQ-II)

The acceptance and action questionnaire (AAQ-II) evaluation is a measure of psychosocial flexibility, defined broadly as the ability to fully contact the present moment and the thoughts and feelings it contains without needless defense (Hayes et al., The Psychological Record 2004; 54:553-78; Bond et al., Behav Ther 2011; 42(4):676-88). There are seven items included in the AAQ-II evaluation, and each response carries a numerical value, ranging from 1-7, wherein 1 is never true, 4 is sometimes true, and 7 is always true. Responses for each item are summed and the total score can be interpreted to determine the psychological flexibility of an individual, noting that a range of 24-28 is generally associated with depression, anxiety and/or related symptoms. In some embodiments, the AAQ-II is utilized to evaluate the treatment of one or more symptoms or signs associated with FM, such as depression, anxiety and/or related symptoms associated with FM, such as pain or sleep disturbance.

In some aspects, the embodiments provided herein include assessment of a change from baseline in acceptance and action questionnaire (AAQ-II) evaluation. In some aspects, the AAQ-II evaluation is completed at one or more of the time points following administration of one or more doses of the psychedelic, such as at one or more of: baseline deep profiling, first dose, second dose, end of therapy deep profiling, 1-month follow-up, 3-month follow-up and/or 6-month follow-up.

In some embodiments, the AAQ-II score for an individual receiving treatment decreases during and/or after treatment. In some aspects, the AAQ-II score decreases by at least at or about 1 point, 2 points, 3 points, 4 points, 5 points, 6 points, 7 points, 8 points, 9 points, 10 points, 15 points, 20 points, 25 points, 30 points, 35 points, 40 points, or 45 points.

h. Life Events Checklist (LEC)

In some aspects, one or more indicators include a life events checklist (LEC). IN some aspects, the life events checklist for DSM-5 (LEC-5) (Weathers et al., 2013; National Center for PTSD, https://www ptsd.va.gov) is used to assess an individual's history and exposure to trauma. Individuals indicate how many of 17 potentially traumatic events (e.g., natural disaster, physical assault) have been experienced personally or witnessed, as well as an additional item assessing any other very stressful event or experience. In some embodiments, the methods or uses involve an LEC evaluation. In some embodiments, the LEC is completed at various time points during the treatment regimen, for example, at deep phenotyping.

In some embodiments, the LEC evaluation is utilized to evaluate an effect of the psychedelic for improving one or more symptoms of FM. In some embodiments, the LEC evaluation is repeated for the individual receiving treatment at various time points during an exemplary treatment regimen, for example, at one or more of: baseline deep profiling, first dose, second dose, end of therapy deep profiling, 1-month follow-up, 3-month follow-up and/or 6-month follow-up. In some embodiments, one or more LEC score for an individual receiving treatment increases during and/or after treatment completion. In some embodiments, the LEC for an individual receiving treatment improves compared to baseline.

i. Avoidance Questionnaire

In some embodiments, an indicator is for evaluation of experiential avoidance (EA). EA is an unwillingness to remain in contact with distressing emotions, thoughts, memories, and physical sensations, and can be associated with the development and maintenance of emotional problems. In some cases, EA can actually exacerbate problems (S. Edwards (2020) Value Living with Fibromyalgia. Doctoral thesis, Florida Institute of Technology). In one study, juvenile subjects with fibromyalgia that were able to think rationally about their pain exhibited lower levels of pain and distress (Libby & Glenwick, 2010, Rehabilitation psychology, 55(2):151; see also S. Edwards (2020) Value Living with Fibromyalgia. Doctoral thesis, Florida Institute of Technology). Thus, reduction in EA in a subject with FM, e.g., reduced from baseline, is associated with, or indicate, with a therapeutic outcome of treatment, such as an improvement or amelioration of the one or more symptoms, an effective therapy or an effective treatment of the disease or condition, such as FM or one or more symptoms thereof. In some embodiments, EA can be measured by any of a variety of known methods (see e.g., Kirk et al. 2021 Behavior Therapy, 52:208-220). In some embodiments, EA is measured by a Multidimensional Experiential Avoidance Questionnaire (MEAQ), which is a 62-item questionnaire. In some embodiments, EA is measured by a Brief Experiential Avoidance Questionnaire (BEAQ), which is a shortened version of MEAQ that is a 15-item short form of the MEAQ.

### 4. Psychedelic Experience and Clinical Activity Assessments

In some aspects, the psychedelic experience and the associated clinical impacts are evaluated, before, during and/or after the administration of the one or more doses of the psychedelic and/or psychotherapy. In some aspects, to evaluate the psychedelic experience and the associated clinical impacts for an individual receiving treatment in accordance with the provided embodiments, the individual is subject to a one or more evaluation to assess the psychedelic experience and clinical activity in response to treatment (Davis et al., J Psychopharmacol 2021; 35(4):437-46). In some embodiments, the evaluation to assess the psychedelic experience and clinical activity in response to treatment includes one or more of: the mystical experience questionnaire (MEQ30) evaluation, the challenging experience questionnaire (CEQ) evaluation, the psychological insight questionnaire (PIQ) evaluation, a qualitative written assessment, and/or the monitor rating scale (MRS) questionnaire. In some embodiments, the psychedelic experience and clinical activity is utilized to evaluate the psychedelic experience of an individual receiving the psychedelic. In some embodiments, one or more evaluation is completed at various time points during an exemplary treatment regimen, for example, at one or more of: first dose, second dose, 1-month follow-up, 3-month follow-up and/or 6-month follow-up.

a. Mystical Experience Questionnaire (MEQ30)

In some aspects, one or more indicators include a mystical experience questionnaire (MEQ). In some embodiments, MEQ can include MEQ30 (derived and abbreviated from the MEQ43), an evaluation completed by the patient, comprising four factors: mystical (e.g., internal unity, external unity, noetic quality, and sacredness scales), positive mood, transcendence of time and space, and ineffability (Barrett et al., J Psychopharmacol 2015; 29(11):1182-90). The MEQ30 predicts persisting therapeutic benefits (e.g., change in attitudes, behavior, and well-being) attributed to psychedelic experiences, serving as a key measure of an individual's mystical experience. There are 30 items included in the MEQ30 evaluation, distributed across the four factors, and each items is rated on a 0-5 point scale, wherein 0 is “none; not at all,” 1 is “so slight cannot decide,” 2 is “slight,” 3 is “moderate,” 4 is “strong (equivalent in degree to any previous strong experience or expectation of this description),” and 5 is “extreme (more than ever before in my life and stronger than 4).” Across these 30 items, the four factors, or sub-categories, comprise different items, wherein mystical consists of items 4-6, 9 14-16, 18, 20, 21, 23-26, and 28; positive mood consists of items 2, 8, 12, 17, 27, and 30; space/time consists of items 1, 7, 11, 13, 19, and 22; and ineffability consists of items 3, 10, and 29. Scores for each item within a sub-category, or across the full MEQ30 set, are summed together and converted to a percentage by dividing by the corresponding point total. A “complete experience” is defined by a MEQ30 score equal to or exceeding 60% (of the total) across all four sub-categories, wherein the complete experience correlates with more significant and/or extended therapeutic benefits.

In some embodiments, the MEQ30 is utilized to evaluate the psychedelic experience of an individual receiving the psychedelic. In some embodiments, the MEQ30 evaluation is completed at various time points during an exemplary treatment regimen, for example, at one or more of: first dose and second dose. In some embodiments, the MEQ30 score for an individual receiving treatment improves during and/or after treatment completion. In some aspects, one or more MEQ30 score improves by at least 1 point, at least 5 points, at least 10 points, at least 15 points, at least 20 points, or at least 30 points compared to baseline.

b. Challenging Experiences Questionnaire (CEQ)

In some aspects, one or more indicators include a challenging experience questionnaire (CEQ). Due to the powerful experience induced by classical hallucinogens (e.g., psilocybin), a patient receiving treatment may endure one or more acute adverse psychological reactions (i.e., a bad trip, or a challenging experience). In some aspects, evidence support that symptoms including affective (panic, depressed mood), cognitive (confusion, feelings of losing sanity), and somatic (nausea, heart palpitation) are possible (Barrett et al., J Psychopharmacol 2016; 30(12):1279-95). Understanding the range and severity of symptoms is useful in tailoring patient care and anticipating patient needs during future treatment sessions. The CEQ addresses seven prominent factors (grief, fear, death, insanity, isolation, physical distress, and paranoia) and provides a phenomenological profile of challenging aspects that a patient has or may experience during treatment (Barrett et al., J Psychopharmacol 2016; 30(12):1279-95). There are 26 items included in the CEQ evaluation, and each response carries a numerical value, ranging from 0-5, wherein 0 is ‘none; not at all’ and 5 is ‘extreme (more than ever before in my life).’ Across these 26 items, the seven factors, or sub-categories, comprise different items, wherein grief consists of items 2, 6, 9, 11, 23, and 25; fear consists of items 4, 7, 14, 21, and 26; death consists of items 16 and 20; insanity consists of items 8, 13, and 19; isolation consists of items 1, 10, and 24; physical distress consists of items 3, 5, 15, 17, and 18; and paranoia consists of items 12 and 22. Responses are summed within each sub-category and converted to a percentage of the total possible points. Further, responses for all items are summed and converted to a percentage of the total possible points.

In some embodiments, the CEQ is utilized to evaluate the psychedelic experience of an individual receiving the psychedelic. In some embodiments, the CEQ evaluation is completed at various time points during an exemplary treatment regimen, for example, at one or more of: first dose and second dose. In some embodiments, the CEQ score for an individual receiving treatment decreases during and/or after treatment completion. In some aspects, one or more CEQ score decreases by at least 0.1%, by at least 0.2%, by at least 0.3%, by at least 0.4%, by at least 0.5%, or more than 0.5% compared to baseline.

c. Psychological Insight Questionnaire (PIQ)

In some aspects, one or more indicators include a psychological insight questionnaire (PIQ). PIQ is a tool for assessing the intensity of a broad range of acute insights (Davis et al., J Psychopharm 2021; 35(4):437-46). The PIQ is a 23-item evaluation comprising two subscales, wherein one assesses insights into avoidance and maladaptive patterns (AMP), while the other assesses goals and adaptive patterns (GAP) (Davis et al., J Psychopharm 2021; 35(4):437-46). Items 1, 3, 5, 7, 9, 10, 12, 14, 15, 17-19, 21, and 23 relate to the AMP subscale, while items 2, 4, 6, 8, 11, 13, 16, 20, and 20 relate to the GAP subscale. Individuals respond to each item using a 6-point scale from 0 to 5, wherein 0 is “no, not at all,” 3 is “moderately,” and 5 is “extremely (more than ever before in my life).” Responses are summed and averaged, for each subscale and in total, with a higher score for each corresponding to higher levels of acute insight experience. PIQ, AMP, and GAP scores correlate well with increases in psychological flexibility, well-being, and long-term positive outcomes.

In some embodiments, the PIQ evaluation is utilized to evaluate the psychedelic experience of an individual receiving the psychedelic. In some embodiments, the PIQ evaluation is repeated for the individual receiving treatment at various time points during an exemplary treatment regimen, for example, at one or more of: first dose, second dose, end of therapy deep profiling, 1-month follow-up, 3-month follow-up and/or 6-month follow-up. In some embodiments, the total PIQ score for an individual receiving treatment decreases during and/or after treatment completion. In some aspects, the PIQ score decreases 1 or more points, 2 or more points, 3 or more points, 4 or more points, 5 or more points, or 10 or more points compared to baseline. In some embodiments, one or more PIQ subscale score for an individual receiving treatment decreases during and/or after treatment completion. In some aspects, one or more PIQ subscale score decreases 1 or more points, 2 or more points, 3 or more points, 4 or more points, 5 or more points, or 10 or more points compared to baseline.

d. Monitor Rating Scale (MRS)

In some aspects, one or more indicators include a monitor rating scale (MRS) questionnaire. In some aspects, the MRS is completed by a clinician and involves rating and scoring dimensions of subject's behavior and/or mood during the treatment session (Griffiths et al., Psychopharmacology Berl 2006; 187(3):268-83). There are 20 items (or dimensions) included in the MRS evaluation, and some dimensions are assigned a numerical value, ranging from 0-4, wherein 0 is ‘none; no effect’ and 4 is ‘peak effect; happening often.’ For other dimensions, scoring is assigned as the total duration (in minutes, across a defined window of time) observed for a patient behavior (e.g., talking with the clinician or total speech). Dimension scores are summed within each sub-category or across all dimensions.

In some embodiments, the MRS is utilized to evaluate the clinical activity of the psychedelic. In some embodiments, the MRS evaluation is completed at various time points during an exemplary treatment regimen, for example, at one or more of: first dose and second dose. In some embodiments, the MRS score for an individual receiving treatment improves during and/or after treatment completion. In some aspects, one or more MRS score improves by at least 1 points, at least 5 points, at least 10 points, at least 15 points, at least 20 points, or at least 30 points compared to baseline.

e. Qualitative (Written) Assessment

A subjective narrative description of the psychedelic experience can provide insight into unique aspects, needs and concerns for specific individuals during and/or after treatment completion. Narrative descriptions are written after each treatment dose session (e.g., first or second dose). After the final dosing session, individuals participate in therapy integration sessions (e.g., one hour each week), wherein discussion of the one or more narrative description helps the individual to gain insight and understanding from their experience. The narrative description is also used to investigate common themes associated with this therapy. Finally, individuals are asked open-ended questions during the 3-month follow-up to gauge persistent changes in behavior, thought patterns, and emotions in response to receiving treatment.

In some embodiments, the qualitative written assessment is utilized to evaluate the psychedelic experience and clinical activity of an individual receiving the psychedelic. In some embodiments, the qualitative written assessment is completed at various time points during an exemplary treatment regimen, for example, at one or more of: after the first dose and after the second dose. In some embodiments, the qualitative written assessment is discussed after assessment. In some embodiments, the qualitative written assessment for an individual receiving treatment improves during and/or after treatment completion. In some aspects, one or more characteristic aspect comprising general spirit, tone, mood, and/or demeanor of the written assessment or reflections thereon is improved.

### B. Clinical Indicators

In some aspects, an individual receiving treatment in accordance with the provided embodiments is subject to one or more evaluation prior to, during, and after treatment, of one or more clinical indicators, such as indicators that are measurable in the clinic and/or using particular instruments. In some aspects, the clinical indicators relate to one or more symptoms of FM, such as sensory measurements, pain or sleep disturbance. In some embodiments, the evaluation of the individual receiving treatment includes a quantitative measure of clinical indicators. In some embodiments, one or more indicators, such as clinical indicators, that are assessed include sensory sensitivity, neuroimaging measurements, and sleep metrics. In some embodiments, the change from baseline of the one or more clinical indicators is evaluated. In some embodiments, the evaluation of the individual receiving treatment comprises evaluation of endpoint analyses.

In some aspects, the clinical indicators relate to one or more symptoms of FM, such as sensory sensitivity and pain assessment. In some aspects, the clinical indicators relate to one or more symptoms of FM, related to sleep. In some aspects, the clinical indicators relate to the changes in the brain in view of the psychedelic experience.

In some embodiments, the evaluation of the individual receiving treatment includes a quantitative measure of clinical indicators, wherein the clinical indicator is measured with one or more methods such as quantitative sensory testing, electroencephalogram (EEG) analysis, functional magnetic resonance imaging (fMRI) analysis, proton magnetic resonance spectroscopy (¹H-MRS), diffusion tensor imaging (DTI), voxel-based morphometry (VBM), and/or wrist actigraphy.

In some embodiments, change from baseline evaluation in sensory sensitivity, neuroimaging measurements, and sleep metrics are measured with one or more method and/or instrument including QST, EEG, fMRI, ¹H-MRS, DTI, VBM, and/or wrist actigraphy. In some embodiments, the indicator evaluation in sensory sensitivity includes a cuff pressure pain assessment and a visual hypersensitivity assessment. In some embodiments, the indicator evaluation in neuroimaging measurements includes an explosive synchronization assessment, a power spectrum analysis, a default mode network analysis, a functional connectivity and activation analysis, a glutamine plus glutamate measurement, a GABA measurement, and a grey matter volume assessment. In some embodiments, the indicator evaluation in sleep metrics includes a sleep onset latency assessment, a wake after sleep onset assessment, a total sleep assessment, and a sleep efficiency assessment. In some embodiments, the indicator evaluation in sensory sensitivity and neuroimaging measurements are completed at various time points during an exemplary treatment regimen, for example, at one or more of: baseline deep profiling and end of therapy deep profiling. In some embodiments, the indicator evaluation in sleep metrics is completed at various time points during an exemplary treatment regimen, for example, at one or more of: baseline deep profiling to preparative therapy and integration to end of therapy deep profiling.

In some aspects, an individual receiving treatment in accordance with the provided embodiments is subject to one or more clinical indicator assessment, wherein the assessment comprises quantitative sensory testing (QST), electroencephalogram (EEG) analysis, functional magnetic resonance imaging (fMRI) analysis, proton magnetic resonance spectroscopy (¹H-MRS), diffusion tensor imaging (DTI), voxel-based morphometry (VBM), wrist actigraphy, and combinations thereof. In some embodiments, the evaluation of the individual receiving treatment includes a quantitative measure of clinical indicators. In some embodiments, the quantified clinical indicator is related to the sensory sensitivity of an individual receiving treatment, including but not limited to a cuff pressure pain assessment and a visual hypersensitivity assessment. In some embodiments, the quantified clinical indicator is related to the neurobiology of an individual receiving treatment, including but not limited to brain activity, functional connectivity, functional activation, and brain morphology. In some embodiments, the quantified clinical indicator is related to aspects of sleep of an individual receiving treatment, including but not limited to sleep onset latency, wake after sleep onset, total sleep time, and sleep efficiency. In some embodiments, the evaluation of the individual receiving treatment includes a quantitative measure of clinical indicators, wherein the clinical indicator is measured with one or more methods including but not limited to quantitative sensory testing (QST), electroencephalogram (EEG) analysis, in functional magnetic resonance imaging (fMRI) analysis, proton magnetic resonance spectroscopy (¹H-MRS), diffusion tensor imaging (DTI), voxel-based morphometry (VBM), wrist actigraphy, and combinations thereof.

### 1. Sensory Sensitivity

In some embodiments, the quantified clinical indicator is related to the sensory sensitivity of an individual receiving treatment. Exemplary clinical indicators include, for example, a cuff pressure pain assessment and a visual hypersensitivity assessment.

In some aspects, an exemplary indicator includes measure of sensory sensitivity. Exploring the sensory sensitivity of an individual experiencing chronic pain (e.g., an individual having FM) can be beneficial for quantifying and better understanding the individual's symptoms and sensitivities (Uddin and MacDermid, Pain Med 2016; 17(9):1694-703). Quantitative sensory testing (QST) is psychophysical testing approach defined as “the determination of thresholds or stimulus response curves for sensory processing under normal and pathological conditions (Arendt-Nielsen and Yarnitsky, J Pain 2009; 10(6):556-72), wherein the stimulus is quantified and used to measure perception (Yarnitsky and Granot, Handb Clin Neurol 2006; 81:397-409). QST is a semi-subjective (e.g., assessing subjective responses) technique, but responses are derived using known stimulus inputs, providing an objective framework for assessing the individual (e.g., before, during, or after therapeutic intervention). Examples of stimulus modalities include electrical, thermal, immersion thermal, mechanical, chemical, and light touch, wherein the pain measurements might include pain threshold/tolerance, temporal summation, conditioned pain modulation, pain rating, pain area mapping, cerebral responses (e.g., EEG, fMRI, PET), or muscle reflexes (Uddin and MacDermid, Pain Med 2016; 17(9):1694-703). All QST procedures have been evaluated for safety and are well tolerated by chronic pain patients (Petersen et al., Pain 2015; 156(1):55-61; Goodin et al., J Pain 2009; 10(2):180-90; Weissman-Fogel et al., J Pain 2009; 10(6):628-36; Price et al., Pain 2002; 99(1-2):49-59). In some embodiments, the evaluation of the individual receiving treatment includes a quantitative measure of clinical indicators. In some embodiments, the quantified clinical indicator is related to the sensory sensitivity of an individual receiving treatment, including but not limited to, a cuff pressure pain assessment and a visual hypersensitivity assessment. In some embodiments, the evaluation is measured with one or more methods including but not limited to quantitative sensory testing, electroencephalogram (EEG) analysis, in functional magnetic resonance imaging and (fMRI) analysis, and combinations thereof. In some embodiments, the QST assessment is performed at various time points during an exemplary treatment regimen, for example, at one or more of: baseline deep phenotyping and end of therapy deep phenotyping.

a. Cuff Pressure Pain

In some cases, large volume, deep muscle pressure sensitivity (Graven-Nielsen et al., Pain 2015; 156(11):2193-2202; Izumi et al., 2014 Pain 155(4):792-800) is assessed using an MRI-compatible rapid cuff inflator (Hokanson, Bellevue, WA) (Loggia et al., Pain 2012; 153(10):2140-51; Polianskis et al., Eur J Pain 2002; 6(6):475-84; Polianskis et al., Eur J Pain 2001; 5(3):267-77). In some aspects, such evaluation includes a cuff pressure pain assessment. An individual receiving treatment is subjected to an ascending series of cuff pressures, starting between 20 mm Hg and 40 mm Hg, and then increasing in 20 mm Hg steps (10 second pressures, 20 second intervals) to tolerance or a maximum of 400 mm Hg. Each pressure is rated after deflation on a 0 to 100 numeric rating scale. Individual-reported pain ratings are used to determine a series of eight tolerable cuff pressures that are delivered in pseudo-randomized order and rated individually on pain intensity and unpleasantness, using the same 0 to 100 scale. Stimulus response curves are constructed for each individual and used for analysis, along with several derived variables (e.g., cuff pain threshold (cPTT), cuff-Pain50, and cuff-tolerance). In addition, tonic pain induced by continuous cuff pressure will be assessed (tonic-cuff). Using a calibrated Pain40-60 pressure for each individual (i.e., pressure that evokes a 40 to 60/100 pain rating) will be applied for 6 to 8 minutes to one gastrocnemius muscle. Pain intensity and unpleasantness ratings will be obtained every 60 seconds. This tonic cuff procedure is also performed during fMRI analysis and EEG analysis.

In some embodiments, a cuff pressure pain is assessed to evaluate an effect of the psychedelic for improving one or more symptoms of FM. In some embodiments, the change from baseline in the cuff pressure pain is utilized to evaluate an effect of the psychedelic for improving one or more symptoms of FM. In some embodiments, the cuff pressure pain assessment is performed prior to and after treatment completion. In some embodiments, the cuff pressure pain assessment is performed at various time points during an exemplary treatment regimen, for example, at one or more of: baseline deep phenotyping and end of therapy deep phenotyping. In some embodiments, a cuff pressure pain for an individual receiving treatment decreases during and/or after treatment completion. In some aspects, the cuff pressure pain decreases by at least 1%, at least 2%, at least 3%, at least 4%, at least 5%, or at least 10% compared to baseline.

b. Visual Hypersensitivity

In addition to painful stimuli hypersensitivity, many people with chronic pain report hypersensitivity to non-painful sensory stimuli, including to auditory, olfactory, and visual stimulation (Lopez-Sola et al., Arthritis Rheumatol 2014; 66(11):3200-9; Wilbarger and Cook, Arch Phys Med Rehabil 2011; 92(4):653-56; Geisser et al., J Pain 2008; 9(5):417-22), indicating a global state of multisensory amplification in centralized pain. To measure non-somatic sensitivity, an individual is presented a series of non-painful yet aversive visual stimuli (Harte et al., Pain 2016; 157(9):1933-45). This task persists for 10 minutes and consists of different visual stimuli presented in an alternating block design. The control stimulus is a fixed crosshair centered in the middle of a solid color background or a black screen, whereas experimental stimulus is a flashing annulus checkerboard. Subjects view the stimuli on a calibrated HD radiological LCD monitor with stimuli being delivered in ascending and random order, adjusted to varying degrees of frequency (i.e., checkerboard flashing only), color, shape, motion, and/or brightness level, and rated on a 0 to 100 numeric rating scale of sensory intensity and unpleasantness. To avoid possible AEs in vulnerable patients, the visual stimulation task is not to be performed if the subject has a history of photo-induced migraines or seizures. These subjects are allowed to complete all other procedures. In some embodiments, a visual hypersensitivity is assessed to evaluate an effect of the psychedelic for improving one or more symptoms of FM.

In some embodiments, the change from baseline in the visual hypersensitivity is utilized to evaluate an effect of the psychedelic for improving one or more symptoms of FM. In some embodiments, the visual hypersensitivity assessment is performed prior to and after treatment completion. In some embodiments, the visual hypersensitivity assessment is performed at various time points during an exemplary treatment regimen, for example, at one or more of: baseline deep phenotyping and end of therapy deep phenotyping. In some embodiments, a visual hypersensitivity for an individual receiving treatment decreases during and/or after treatment completion. In some aspects, the visual hypersensitivity decreases by at least 1%, at least 2%, at least 3%, at least 4%, at least 5%, or at least 10% compared to baseline.

### 2. Sleep Metrics

In some embodiments, the quantified clinical indicator is related to aspects of sleep of an individual receiving treatment, such as sleep onset latency, wake after sleep onset, total sleep time, and sleep efficiency. In some aspects, sleep metrics can be measured using methods such as wrist actigraphy.

a. Wrist Actigraphy

Wrist actigraphy is a method of tracking sleep and sleep-related movement in the ambulatory environment or at home, wherein an actigraph devices is worn on the wrist and recorded information is used to computationally estimate sleep parameters (Martin and Hakim, Chest 2011; 139(6):1514-27). An individual receiving treatment is provided a wrist actigraphy device. In some aspects, the device is selected from among one or more of: an Actiwatch Spectrum (Philips Respironics), a PROdiary (CamNtech) and the GT9X-BT actiwatch (Actigraph). The wrist actigraphy device is used to collect objective measures for the individual including but not limited to total sleep time (i.e., sleep duration), wake after sleep onset (i.e., sleep continuity), and sleep timing (e.g., sleep onset and wake time). Individuals also complete two sleep logs (0 to 10 numeric rating scale for daily sleep quality, time in bed, number of wake times, final wake time, estimated sleep onset), via the actigraphy device and in a diary every morning (Burgess H, J Biol Rhythms 2010; 25(6):460-8; Burgess et al., J Clin Endocrinol Metab; 2010 95(7):3325-31). Data extraction, cleaning, and analysis is performed using the proprietary algorithm provided by the manufacturer.

In some embodiments, a wrist actigraphy is assessed to evaluate an effect of the psychedelic for improving one or more symptoms of FM and sleep-related symptoms. In some embodiments, the change from baseline in the wrist actigraphy is assessed. In some embodiments, the wrist actigraphy assessment is performed prior to and after treatment completion. In some embodiments, the wrist actigraphy assessment is performed at various time points during an exemplary treatment regimen, for example, at one or more of: baseline deep phenotyping, preparative therapy, integration sessions, and end of therapy deep phenotyping. In some embodiments, one or more aspect of the wrist actigraphy assessment for an individual receiving treatment improves during and/or after treatment completion. In some aspects, the one or more aspect improves by at least 1%, at least 2%, at least 3%, at least 4%, at least 5%, or at least 10% compared to baseline.

### (i) Sleep Onset Latency

Sleep onset latency (SOL) is an important parameter in a sleep analysis, wherein sleep latency is defined as the duration of time between “lights out” as an individual attempts to sleep, until the time individual actually falls asleep, as determined by changes in EEG signal and behavioral parameters changes consistent with sleep (Shrivastava et al., J Community Hosp Intern Med Perspect 2014; 4(5):24983).

In some embodiments, a SOL assessment is performed prior to and after treatment completion. In some embodiments, the SOL assessment is performed at various time points during an exemplary treatment regimen, for example, at one or more of: baseline deep phenotyping, preparative therapy, integration sessions, and end of therapy deep phenotyping. In some embodiments, a SOL measurement for an individual receiving treatment improves during and/or after treatment completion. In some aspects, the SOL assessment improves by at least 1%, at least 2%, at least 3%, at least 4%, at least 5%, or at least 10% compared to baseline.

### (ii) Wake After Sleep Onset

Wake after sleep onset (WASO) refers to periods of wakefulness occurring after defined sleep onset. WASO measures wakefulness occurring after sleep onset and is a reliable reflection of sleep fragmentation (Shrivastava et al., J Community Hosp Intern Med Perspect 2014; 4(5):24983).

In some embodiments, a WASO assessment is performed prior to and after treatment completion. In some embodiments, the WASO assessment is performed at various time points during an exemplary treatment regimen, for example, at one or more of: baseline deep phenotyping, preparative therapy, integration sessions, and end of therapy deep phenotyping. In some embodiments, a WASO measurement for an individual receiving treatment improves during and/or after treatment completion. In some aspects, the WASO measurement decreases by at least 1%, at least 2%, at least 3%, at least 4%, at least 5%, or at least 10% compared to baseline.

(iii) Total Sleep

Total sleep time refers to the total sleep time from sleep onset to sleep offset. In some aspects, the total sleep time is a summation of non-rapid eye movement stages 1, 2, and 3 sleep, as well as rapid eye movement (REM) sleep (Shrivastava et al., J Community Hosp Intern Med Perspect 2014; 4(5):24983). Total sleep time is collected in combination with other parameters to assess sleep quality and sleep-related disorders. Low total sleep time can be indicative of a medical or sleep disorder, whereas long total sleep time may indicate prior sleep deprivation, a medical conditions, or effects of current medications. In some embodiments, a total sleep assessment is performed prior to and after treatment completion. In some embodiments, the total sleep assessment is performed at various time points during an exemplary treatment regimen, for example, at one or more of: baseline deep phenotyping, preparative therapy, integration sessions, and end of therapy deep phenotyping.

In some embodiments, the total sleep measurement for an individual receiving treatment improves during and/or after treatment completion. In some aspects, the total sleep measurement improves by at least 1%, at least 2%, at least 3%, at least 4%, at least 5%, or at least 10% compared to baseline.

### (iv) Sleep Efficiency

Sleep efficiency refers to percentage of total time in bed actually spent in sleep, wherein total sleep is divided by the total time in bed and multiplied by 100. Sleep efficiency provides a general assessment of sleep quality, but it does not account for high levels of sleep fragmentation (Shrivastava et al., J Community Hosp Intern Med Perspect 2014; 4(5):24983).

In some embodiments, a sleep efficiency assessment is performed prior to and after treatment completion. In some embodiments, the sleep efficiency assessment is performed at various time points during an exemplary treatment regimen, for example, at one or more of: baseline deep phenotyping, preparative therapy, integration sessions, and end of therapy deep phenotyping. In some embodiments, a sleep efficiency measurement for an individual receiving treatment increases during and/or after treatment completion. In some aspects, the sleep efficiency measurement increases by at least 1%, at least 2%, at least 3%, at least 4%, at least 5%, or at least 10% compared to baseline.

### 3. Neuroimaging Measurements

In some embodiments, the quantified clinical indicator include those related to the neurobiology of an individual receiving treatment, such as brain activity, functional connectivity, functional activation, and brain morphology.

Research aimed at defining and leveraging neurophysiologic clinical indicators, including but not limited to brain activity, functional connectivity, functional activation, and brain morphology, has intensified with the advent of new methodologies, technical advances, and the rapidly evolving field of neuroinformatic, a key factor in the subsequent analysis of large datasets from population size cohorts (Roalf and Gur, Neuropsychology 2017; 31(8):954-71; Woo et al., Nat Neurosci 2017; 20(3):365-77; Horien et al., Nat Hum Behav 2021; 5(2):185-93. Advances like these have provided new neurological tools to measure brain-specific changes more quantitatively during development, aging, learning, disease, and in response to acute stimuli, including small-molecule compounds and/or biomolecules (Borsook et al., Nat Rev Drug Discov 2006; 5(5):411-24; Matthews and Hampshire, Neuron 2016; 91(3):511-28; Carmichael et al., Drug Discov Today 2018; 23(2):333-48). Although the benefits of psychedelic intervention has been observed anecdotally and qualitatively for centuries, the recent push for clinical acceptance has motivated the development and implementation of more rigorous and quantitative approaches.

a. Brain Activity

Brain activity can be routinely monitored using non-invasive methods such as EEG and fMRI (Abreu et al., Front Hum Neurosci 2018; 12:29). An EEG records electrical activity via the scalp on the millisecond timescale. In some aspects, the recorded signal represents the macroscopic activity of the brain. An fMRI detects changes in blood flow within the brain. In some aspects, these changes correspond to regional and site-specific changes in activity (Carhart-Harris et al., Sci Rep 2017; 7(1):13187). 1H-MRS is another non-invasive neuroimaging technique that enables the detection, identification, and quantification of metabolites and biochemical compounds in the brain tissue (Castillo et al., AJNR Am J Neuroradiol 1996; 17(1):1-15; Sidek et al., Eur Radiol 2016; 26(12):4404-12). Finally, diffusion tensor imagining (DTI) is newer method for anatomical mapping and characterization of microstructures in the brain (Alexander et al., Neurotherapeutics 2007; 4(3):316-29), making it useful for bridging the gap between EEG data and fMRI data, collectively enhancing the value of the neurological data set. In some aspects, an individual receiving treatment in accordance with the provided embodiments is subject to one or more evaluation prior to, during, and after treatment. In some embodiments, the evaluation of the individual receiving treatment comprises quantification of clinical indicators. In some embodiments, the methods or uses involve a change from baseline evaluation in neuroimaging measurements.

In some embodiments, change from baseline evaluation in neuroimaging measurements are measured with one or more method and/or instrument including QST, EEG, fMRI, ¹H-MRS, DTI, VBM, and combinations thereof. In some embodiments, the change in baseline evaluation in neuroimaging measurements includes an explosive synchronization assessment, a power spectrum analysis, a default mode network analysis, a functional connectivity and activation analysis, a glutamine plus glutamate measurement, a GABA measurement, and a grey matter volume assessment. In some embodiments, the change in baseline evaluation in neuroimaging measurements are completed at various time points during an exemplary treatment regimen, for example, at one or more of: baseline deep profiling and end of therapy deep profiling.

### (i) Explosive Synchronization and QST

In some aspects, reports indicate chronic pain originates from disruption or dysfunction of complex neural networks, specifically an imbalance in excitatory and inhibitory brain activity causing an unstable neural network sensitized to external pain stimuli (Crofford L, Trans Am Clin Climatol Assoc 2015; 126:167-83). Explosive synchronization (ES) is a phenomenon wherein small increases in stimulation strength applied to a network can lead to an abrupt state transition through global network synchronization (Arenas et al., Phys Rep 2008; 469:93; Wang et al., Sci Rep 2017; 7(1):561). Using an electroencephalogram (EEG) analysis, various aspects of the brain activity can be collected to investigate ES events (Kim et al., Front Comput Neurosci 2016; 10:1).

In some aspects, the EEG analysis records high-resolution temporal brain activity (Michel and Brunet, Front Neurol 2019; 10:325), measuring direct electrical signals via the scalp and providing millisecond timescale information, compared seconds and minutes needed for other preferred methodologies. EEG data can be collected from multiple different states (e.g., during rest, during tonic cuff pressure, during visual stimulus) to capture objective response signals and changes in brain activity.

In some embodiments, an EEG is assessed to evaluate an effect of the psychedelic for improving one or more symptoms of FM. In some embodiments, the EEG assessment additionally comprising one or more sensory stimulus is utilized. In some embodiments, the change from baseline in an ES measurement is utilized. In some embodiments, the change from baseline in a resting EEG measurement is utilized. In some embodiments, the change from baseline in the resting EEG measurement during and/or after a sensory stimulus is utilized. In some embodiments, the EEG assessment is performed prior to and after treatment completion. In some embodiments, the EEG assessment additionally comprising one or more sensory stimulus is performed prior to and after treatment completion. In some aspects, the sensory stimulus comprises cuff pressure pain and visual paradigms. In some embodiments, EEG assessment is performed at various time points during an exemplary treatment regimen, for example, at one or more of: baseline deep phenotyping and end of therapy deep phenotyping.

In some embodiments, EEG assessment additionally comprising one or more sensory stimulus is performed at various time points during an exemplary treatment regimen, for example, at one or more of: baseline deep phenotyping and end of therapy deep phenotyping. In some embodiments, the ES measurement for an individual receiving treatment improves during and/or after treatment completion. In some aspects, the ES measurement improves by at least 1%, at least 2%, at least 3%, at least 4%, at least 5%, or at least 10% compared to baseline. In some embodiments, the resting EEG measurement for an individual receiving treatment improves during and/or after treatment completion. In some aspects, the resting EEG measurement improves by at least 1%, at least 2%, at least 3%, at least 4%, at least 5%, or at least 10% compared to baseline. In some embodiments, the EEG measurement additionally comprising one or more sensory stimuli for an individual receiving treatment improves during and/or after treatment completion. In some aspects, the EEG measurement additionally comprising one or more sensory stimuli improves by at least 1%, at least 2%, at least 3%, at least 4%, at least 5%, or at least 10% compared to baseline.

### (ii) Default Mode Network, Functional Connectivity, and Sensory Testing

In some aspects, functional connectivity MRI (fMRI) focuses on the default mode network (DMN) and salience network (SLN). The DMN is a constellation of brain regions engaged in self-referential cognition, which are “deactivated” during externally focused tasks (Buckner and Vincent, Neuroimage 2007; 37(4):1091-99; Fox and Raichle Nat Rev Neurosci 2007; 8(9):700-11). Patients with more centralized pain display increased connectivity between the DMN and the insula (an SLN region) which is diminished with successful treatment (Napadow et al., Arthritis Rheum 2012; 64(7):2398-2403). Whole-brain BOLD functional images are acquired while subjects rest comfortably in the scanner with eyes closed for about 8 minutes at the beginning of the scan session to obtain baseline measures of functional brain connectivity. Immediately following, subjects undergo an fMRI scan with sensory stimuli (e.g., tonic cuff pressure pain) to evaluate functional brain connectivity response to deep pain. Pressure intensity is set to each subject's Pain 30 to 50 level, as determined during the behavioral visit, and applied for about 8 minutes. The pressure level is verified prior to the scan initiation and re-calibrated in the event that it evokes more or less pain than anticipated. Pain intensity and unpleasantness ratings are obtained at certain intervals during the test, up to three times and following stimulus presentation.

In some embodiments, an fMRI is assessed to evaluate an effect of the psychedelic for improving one or more symptoms of FM. In some embodiments, the fMRI assessment additionally comprising one or more sensory stimulus is utilized. In some embodiments, the fMRI assessment additionally comprising one or more sensory stimulus is utilized. In some aspects, the sensory stimulus is applied at one or more intensity. In some embodiments, the change from baseline in the default mode network is utilized. In some embodiments, the change from baseline in the connectivity of the default mode network is utilized. In some embodiments, the change from baseline in the default mode connectivity is utilized.

In some embodiments, the functional brain connectivity evaluation is completed in the resting state to obtain a baseline measurement. In some embodiments, the functional brain connectivity evaluation is completed during one or more sensory stimulus. In some embodiments, the functional brain connectivity evaluation is completed during one or more sensory stimulus. In some aspects, the sensory stimulus is applied at one or more intensity. In some embodiments, the sensory stimulus comprises cuff pressure pain and visual stimuli. In some embodiments, the functional brain connectivity evaluation is completed during tonic cuff pressure pain, wherein a response to pain is recorded. In some embodiments, the functional brain connectivity evaluation is completed during visual stimuli, wherein a response to visual cues is recorded.

In some embodiments, the resting state functional brain connectivity assessment is performed prior to and after treatment completion. In some embodiments, the tonic state functional brain connectivity assessment is performed prior to and after treatment completion. In some embodiments, the resting state functional brain connectivity assessment is performed at various time points during an exemplary treatment regimen, for example, at one or more of: baseline deep phenotyping and end of therapy deep phenotyping. In some embodiments, the tonic state functional brain connectivity assessment is performed at various time points during an exemplary treatment regimen, for example, at one or more of: baseline deep phenotyping and end of therapy deep phenotyping. In some embodiments, the resting state functional brain connectivity for an individual receiving treatment improves during and/or after treatment completion. In some aspects, the resting state functional brain connectivity improves by at least 1%, at least 2%, at least 3%, at least 4%, at least 5%, or at least 10% compared to baseline. In some embodiments, the tonic state functional brain connectivity for an individual receiving treatment improves during and/or after treatment completion. In some aspects, the tonic state functional brain connectivity improves by at least 1%, at least 2%, at least 3%, at least 4%, at least 5%, or at least 10% compared to baseline.

(iii) Glutamine plus Glutamate (Glx), Gamma Aminobutyric Acid GABA) Levels

In some cases, using proton magnetic resonance spectroscopy (¹H-MRS), reports indicate that changes in levels of glutamine plus glutamate (Glx) and gamma aminobutyric acid (GABA) within specific regions of the brain (e.g., the posterior and anterior insula) are strongly correlated with improvements in pain, wherein reductions in clinical pain is associated with reduced Glx levels and elevated GABA levels (Mawla et al., Arthritis Rheumatol 2021; 73(7):1318-28; Harris et al., Arthritis Rheum 2009; 60(10):3146-52; Harris et al., Arthritis Rheum 2008; 58(3):903-7). ¹H-MRS provides metrics amenable to longitudinal studies, wherein high-resolution anatomical scans isolate identical brain structures within individuals over time thus minimizing error that otherwise would occur because of slight differences in voxel location from one evaluation to the next. ¹H-MRS is directed to the right posterior insula cortex both prior to and after evoked cuff pressure pain stimulus. Since right and left sides of the brain have been shown to be involved in chronic FM pain (Harris et al., Arthritis Rheum 2009; 60(10):3146-52; Harris et al., Arthritis Rheum 2008; 58(3):903-7), both sides will be explored using this approach. Individuals receiving treatment are at rest during the ¹H-MRS session, wherein one or more ¹H-MRS assessment is integrated into the fMRI neuroimaging workflow.

In some embodiments, a Glx is assessed to evaluate an effect of the psychedelic for improving one or more symptoms of FM, wherein Glx is glutamine plus glutamate. In some embodiments, a GABA is assessed. In some embodiments, a ¹H-MRS is assessed. In some embodiments, the ¹H-MRS is assessed. In some aspects, the ¹H-MRS assessment is completed before one or more sensory stimulus is evoked. In some embodiments, the ¹H-MRS is assessed. In some aspects, the ¹H-MRS assessment is completed after one or more sensory stimulus is evoked. In some embodiments, the change from baseline in the level of Glx is utilized.

In some embodiments, the change from baseline in the level of GABA is utilized to evaluate an effect of the psychedelic for improving one or more symptoms of FM. In some embodiments, the ¹H-MRS assessment is done for one or more region of brain comprising the right posterior insula cortex. In some embodiments, the Glx assessment is performed at various time points during an exemplary treatment regimen, for example, at one or more of: baseline deep phenotyping and end of therapy deep phenotyping. In some embodiments, the GABA assessment is performed at various time points during an exemplary treatment regimen, for example, at one or more of: baseline deep phenotyping and end of therapy deep phenotyping. In some embodiments, the ¹H-MRS assessment is performed at various time points during an exemplary treatment regimen, for example, at one or more of: baseline deep phenotyping and end of therapy deep phenotyping. In some embodiments, the level of Glx for an individual receiving treatment decreases during and/or after treatment completion. In some aspects, the level of Glx decreases by at least 1%, at least 2%, at least 3%, at least 4%, at least 5%, or at least 10% compared to baseline. In some embodiments, the level of GABA for an individual receiving treatment decreases during and/or after treatment completion. In some aspects, the level of GABA decreases by at least 1%, at least 2%, at least 3%, at least 4%, at least 5%, or at least 10% compared to baseline. In some embodiments, a ¹H-MRS signal for an individual receiving treatment improves during and/or after treatment completion. In some aspects, the ¹H-MRS signal improves by at least 1%, at least 2%, at least 3%, at least 4%, at least 5%, or at least 10% compared to baseline.

### (iv) Diffusion Tensor Imaging

Diffusion tensor imaging (DTI) is another neuroimaging technique, leveraging the fMRI platform and used for characterizing small changes in brain structure during neuropathology and treatment (Alexander et al., Neurotherapeutics 2007; 4(3):316-29; Soares et al., Fron Neurosci 2013; 7:31). Here, DTI data is collected during the fMRI neuroimaging procedures to aid with spatial mapping of EEG data. Unique structural brain networks are generated for each individual receiving treatment to better inform prior and subsequent EEG analyses.

In some embodiments, a DTI is assessed to evaluate an effect of the psychedelic for improving one or more symptoms of FM. In some embodiments, the DTI is assessed to supplement or enhance other neuroimaging data for evaluating the effect of the psychedelic. In some embodiments, the DTI is assessed to supplement or enhance EEG data for evaluating the effect of the psychedelic. In some embodiments, the DTI assessment is performed at various time points during an exemplary treatment regimen, for example, at one or more of: baseline deep phenotyping and end of therapy deep phenotyping.

b. Brain Morphology

### (i) Grey Matter Volume

Grey matter is the area of the brain comprising most of its neuronal cell bodies (Lee et al., Sci Total Environ 2020; 707:135603; Chiao et al., Methods Mol Biol 2020; 2092:65-75), and gray matter volume is associated with proper cognitive performance, wherein decreased grey matter volume correlates with cognitive impairment (Spulber et al., Curr Alzheimer Res 2012; 9(4):516-24; Valdes et al., Neuroimage Clin 2020; 25:102158; Dicks et al., Neuroimage Clin 2019; 22:101786). In some aspects, voxel-based morphometry (VBM) is a neuroimaging technique that quantifies focal differences in brain anatomy, wherein images of core components within the brain (e.g., grey matter, white matter, and cerebrospinal fluid) are anatomically normalized and processed according to various computational algorithms and statistics (Wright et al., Schizophr Res 1999; 35(1):1-14; Ashburner and Friston Neuroimage 2000; 11(6 Pt 1):805-21). VBM thus enables global and regional volumetric comparisons between different groups (Andujar et al., Brain Sci 2021; 11(8):999).

In some embodiments, a grey matter volume is utilized to evaluate an effect of the psychedelic for improving one or more symptoms of FM. In some embodiments, a VBM is assessed. In some embodiments, the change from baseline in the grey matter volume is utilized. In some embodiments, the VBM assessment is performed at various time points during an exemplary treatment regimen, for example, at one or more of: baseline deep phenotyping and end of therapy deep phenotyping. In some embodiments, the grey matter volume for an individual receiving treatment increases during and/or after treatment completion. In some aspects, the grey matter volume increases by at least 1%, at least 2%, at least 3%, at least 4%, at least 5%, or at least 10% compared to baseline.

### C. Exemplary Monitoring and Evaluation

In some aspects, an individual receiving treatment in accordance with the provided embodiments is subject to one or more evaluation prior to, during, and after treatment, such as administering one or more doses of the psychedelic, such as psilocybin or a metabolite thereof. In some embodiments, the one or more evaluation of the individual receiving treatment comprises one or more subject-reported evaluation, one or more observer-rated evaluation, one or more quantified clinical indicator, and combinations thereof. In some embodiments, the one or more evaluation of the individual receiving treatment comprises evaluation of primary and/or secondary endpoints. In some embodiments, the evaluation of the individual receiving treatment comprises evaluation of secondary effect endpoints.

In some embodiments, the methods or uses involve an evaluation of change from baseline in a 2016 FM survey questionnaire (FSQ) assessment, a coping strategies questionnaire (CSQ) assessment, a PainDETECT assessment, a positive and negative affect score (PANAS) assessment, a PROMIS-29+2 Profile v2.1 multi-domain assessment, a patient acceptable symptom state (PASS) questionnaire, a mystical experience questionnaire (MEQ30), a challenging experiences questionnaire (CEQ), a psychological insight questionnaire (PIQ), and/or a monitor rating scale (MRS) assessment. In some embodiments, the evaluation of the individual receiving treatment includes a qualitative written assessment of treatment experiences, opinions, and insights. In some embodiments, the FSQ assessment is completed at various time points during an exemplary treatment regimen, for example, at one or more of: screening, baseline deep profiling, end of therapy deep profiling, 1 month follow-up, 3 month follow-up, 6 month follow-up.

In some embodiments, the CSQ assessment, the PainDETECT assessment, and the positive and negative affect score (PANAS) assessments are completed at various time points during an exemplary treatment regimen, for example, at one or more of: baseline deep profiling, end of therapy deep profiling, 1 month follow-up, 3 month follow-up, 6 month follow-up. In some embodiments, the PROMIS-29+2 Profile v2.1 multi-domain assessment are completed at various time points during an exemplary treatment regimen, for example, at one or more of: baseline deep profiling, first dose, second dose, end of therapy deep profiling, 1 month follow-up, 3 month follow-up, 6 month follow-up. In some embodiments, the PROMIS-29+2 Profile v2.1 multi-domain assessment and the PASS questionnaire are completed at various time points during an exemplary treatment regimen, for example, at one or more of: baseline deep profiling, first dose, second dose, end of therapy deep profiling, 1 month follow-up, 3 month follow-up, 6 month follow-up. In some embodiments, the MEQ30, the CEQ, the PIQ, and the MRS assessments are completed at various time points during an exemplary treatment regimen, for example, at one or more of: first dose and second dose. In some embodiments, the qualitative written assessment is completed at various time points during an exemplary treatment regimen, for example, at one or more of: first dose, second dose, 1 month follow-up, 3 month follow-up, 6 month follow-up.

An exemplary scheme of monitoring and evaluation in accordance with the provided embodiments is listed in Table 1. In some aspects, the monitoring and evaluation includes subject-reported and observer-rated outcomes, quantification of clinical indicators, and combinations thereof.

TABLE 1
Exemplary Monitoring and Evaluation Scheme

| | Procedures | | | | | | | | | |
|---|---|---|---|---|---|---|---|---|---|---|
| | | | | | | | | | End of | |
| | | Baseline | | Administration | | | | | Therapy^{c} | |
| | | Deep | | | Between | | After | | Deep | Follow- |
| | | Pheno- | Preparation | Dose 1 | Dose 1 & 2 | Dose 2 | Dose 2 | Integration^{b} | Pheno- | up3 and |
| | Screening ^{a} | typing | Therapy | (8 h) | (2 h) | (8 h) | (2 h) | (2 h) | typing | 6 months |
| Visit Number | 1 | 2 | 3, 4 | 5 | 6, 7 | 8 | 9 | 10, 11, | 13 | 14 |
| | | | | | | | | and 12 | | and 15 |
| Day Number | Up to −28 | 1 to 7 | 11 and | 22 ± 3 | 23 and | 36 ± 3 | 37 ± 3 | 43, 50, | 64 ± 3 | 120 and |
| | | | 18 ± 3 | | 29 ± 3 | | | and 57 ± 3 | | 204 ± 7 |
| Informed consent | X | | | | | | | | | |
| Inclusion/exclusion | X | | | | | | | | | |
| criteria | | | | | | | | | | |
| Demographics | X | | | | | | | | | |
| Medical history | | | | | | | | | | |
| SCID-5 and | | | | | | | | | | |
| SCID-5-PD | | | | | | | | | | |
| Prior/concomitant | X | X | X | X | X | X | X | X | X | X |
| medications | | | | | | | | | | |
| 12-lead | X | | | | | | | | | |
| electrocardiogram^{d} | | | | | | | | | | |
| Pregnancy test for | X | X | | X | | X | | | X | |
| WOCBP^{e} | | | | | | | | | | |
| Urine drug/breath | X | X | | X | | X | | | X | |
| alcohol test^{f} | | | | | | | | | | |
| PHQ-8^{g} | X | | | | | | | | | |
| Vital signs^{h} | X | X | | X | | X | | | X | |
| C-SSRS | X | X | X | X | X | X | X | X | X | X |
| 2016 FM Survey | X | X | | | | | | | X | X |
| Criteria | | | | | | | | | | |
| STOP-Bang | | X | | | | | | | | |
| Life Events | | | | | | | | | | |
| Checklist | | | | | | | | | | |
| CMSI | | | | | | | | | | |
| Sleep | | X | | | | | | | X | X |
| Disturbance (8b) | | | | | | | | | | |
| Sleep-related | | | | | | | | | | |
| Impairment (4a) | | | | | | | | | | |
| CPAQ-8 | | | | | | | | | | |
| painDETECT | | | | | | | | | | |
| PANAS short | | | | | | | | | | |
| form (state) | | | | | | | | | | |
| Catastrophizing | | | | | | | | | | |
| PI-NRS daily diaryi | | X | X | X | X | X | X | X | X | |
| Adverse event | | X^{k} | X | X | X | X | X | X | X | |
| reportingj | | | | | | | | | | |
| Quantitative | | X | | | | | | | X | |
| sensory test | | | | | | | | | | |
| fMRI | | | | | | | | | | |
| Electroencephalogram | | | | | | | | | | |
| Wrist actigraphy | | X | X | | | | | X | X | |
| PROMIS-29 + 2 | | X | | X | | X | | | X | X |
| psilocybin | | | | X | | X | | | | |
| administration | | | | | | | | | | |
| Audio/video record of | | | | X | | X | | | | |
| dose | | | | | | | | | | |
| Monitor rating formb | | | | X | | X | | | | |
| Mystical | | | | X^{l} | | X^{l} | | | | |
| Experiences Q30 | | | | | | | | | | |
| Challenging | | | | | | | | | | |
| Experiences Q27 | | | | | | | | | | |
| Psychological Insight | | | | X^{l} | | X^{l} | | | X | X |
| Q | | | | | | | | | | |
| PGI-C | | | | X | X | X | X | X | X | X |
| Qualitative (written) | | | | X | | X | | | | X^{m} |
| assessment | | | | | | | | | | |

AE = adverse event; CMSI = Complex Multi-symptom Inventory; CPAQ-8 = Chronic Pain Acceptance Questionnaire 8; C-SSRS = Columbia Suicide Severity Rating Scale; FM = fibromyalgia; fMRI = functional connectivity magnetic resonance imaging; PANAS = Positive and Negative Affect Schedule; PGI-C = Patient Global Impression of Change; PI-NRS = pain intensity numeric rating scale; Q = questionnaire; PHQ-8 = Patient Health Questionnaire depression scale; PROMIS-29 + 2 = Patient Reported Outcomes Measurement Information System 29-item profile measure plus 2 cognitive functioning items; SAE = serious adverse event; SCID-5 = Structured Clinical Interview for Diagnostic and Statistical Manual for Mental Disorders 5^{th} Edition; SCID-5-PD = Structured Clinical Interview for Diagnostic and Statistical Manual for Mental Disorders-5for Personality Disorders; WOCBP = women of childbearing potential.
Note:
Assessments scheduled on a day of study drug administration should be performed prior to study drug dosing unless otherwise specified.
^{a} Informed consent must be obtained before any study-specific screening assessments are performed. Screening assessments are to be performed within 28 days preceding Dose 1.
^{b}The Monitor Rating Form will be used by the same 2 therapists to provide ratings from Dose 1 up to 3 weeks post-dose that focus on integration.
^{c}Participants who do not complete both doses of psilocybin will return to the clinic within 28 days after their last dose of study intervention for an End of Therapy Visit.
^{d}Participants should be supine and rested for at least 5 minutes before the ECG is recorded.
^{e}A urine pregnancy test will be performed and results determined locally to exclude pregnant patients.
^{f}Urine drug and breath alcohol testing will be performed and results determined locally
^{g}A result of “severe depression” on PHQ-8 will exclude the participant from the study.
^{h}Includes heart rate, systolic and diastolic blood pressure preferably with an automated device. Participants should be supine and rested for at least 5 minutes in a quietsetting before testing.
iPI-NRS pain diary is to be completed daily, by the participant and checked for completeness by study site staff at each on-site visit indicated.
jAll AEs and SAEs are to be recorded at the times indicated regardless off attribution.
^{k}Medical occurrences that begin before Baseline but after obtaining informed consent will be recorded as medical history, not as AEs.
^{l}To be performed approximately 7 hours after dosing.
^{m}Qualitative written assessment is to be completed at Visit 14 only.

### D. Monitoring of Treatment: Adverse Events

In some embodiments, the methods or uses involve monitoring for adverse events (AEs). In some embodiments, adverse events (AEs) or one or more parameters or indicators associated with potential adverse events are monitored or assessed, before, during or after administration of the psychedelic and/or psychotherapy.

In some embodiments, psychological support providers, observers or session monitors are trained to reassure the subjects if they experience psychological distress, such as one or more of acute anxiety, agitation, paranoia, and panic. In some embodiments, in the unlikely event that psychological distress does not respond to reassurance, or if a subject experiences an adverse event, one of the session monitors contact a physician. In some embodiments, the physician uses their clinical judgement to determine the most appropriate medical intervention.

In some embodiments, AEs are monitored throughout the treatment regimen, for example, at every visit. In some embodiments, observer(s) are available throughout the course of the treatment regimen for emergent AEs. In some embodiments, throughout the administration period, a psychological support provider, e.g., a therapist, assesses the presence and intensity of subject behaviors, symptoms, and non-verbal cues, which includes questions about the presence/intensity of behaviors, signs, and reported symptoms, such as peacefulness, yawning, nausea/vomiting, quantity of speech, anxiety, sleepiness, crying, restlessness, visual changes, euphoria, and feelings of unreality. Individuals complete assessments of mood and safety at the end of the administration sessions. In some embodiments, any of the parameters or indicators described herein, can be used to monitor or assess potential AEs, and the treatment can be adjusted, paused or halted accordingly.

In some aspects, if an individual exhibits an increased risk of harm to self or others, or any other sign that continuing the treatment regimen would present undue risks, the second dose is not administered. In some cases, individuals do not receive a second dose if they have serious adverse events such as psychosis or hypertension. In some aspects, female subject who becomes pregnant during the treatment regimen discontinues the treatment regimen.

In some aspects, clinical studies involving the administration of psychedelics in some cases report similar safety profiles, with both psychological and physical adverse events reported. Oral psilocybin is well tolerated in healthy individuals. No drug-related serious adverse events (SAEs) have been reported. Overall, the most commonly adverse events associated with psilocybin administration were psychological in nature and include anxiety, the induction of negative emotional states and paranoid/delusional thinking during psilocybin sessions, as well as less frequent reports of Hallucinogen Persisting Perception Disorder (HPPD) (Johnson et al., J Psychopharmacol 2008; 22(6):603-20; Tyls et al., European Neuropsychopharmacology 2014; 24(3), 342-56). Rates of prolonged psychiatric symptoms of any kind following psilocybin exposure in healthy study subjects are estimated to be 0.08-0.09%.

In some embodiments, an oral benzodiazepine (e.g., lorazepam) is administered to treat panic or anxiety. In some embodiments, an oral antipsychotic (e.g., risperidone) is available for on-site treatment of psychosis or severe agitation. Common physical effects of psilocybin administration include increased blood pressure (BP) and heart rate (HR), gastrointestinal (nausea, vomiting, and diarrhea), and mild headache.

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

In some aspects, the term “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

In some aspects, integers, steps, or elements of the technology recited herein as singular integers, steps or elements clearly encompass both singular and plural forms of the recited integers, steps or elements.

In some contexts, the terms ‘a’ and ‘an’ are used to refer to one or more than one (i.e., at least one) of the grammatical object of the article. By way of example, reference to ‘an element’ means one element, or more than one element.

In some contexts, the term ‘about’ means that reference to a figure or value is not to be taken as an absolute figure or value, but includes margins of variation above or below the figure or value in line with what a skilled person would understand according to the art, including within typical margins of error or instrument limitation. In some cases words, ‘about’ is understood to refer to a range or approximation that a person or skilled in the art would consider to be equivalent to a recited value in the context of achieving the same function or result.

In some contexts, the terms “treating”, “treatment” and “therapy” refer to curative therapy, prophylactic therapy, palliative therapy and preventative therapy. Thus, in the context of the present disclosure the term “treating” encompasses curing, ameliorating, or tempering the severity of a psychological condition or one or more of its associated symptoms. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. The terms do not imply complete curing of a disease or complete elimination of any symptom or effect(s) on all symptoms or outcomes.

In some contexts, the terms “therapeutically effective amount” or “pharmacologically effective amount” or “effective amount” refer to an amount of an agent sufficient to produce a desired therapeutic or pharmacological effect in the subject being treated. The terms are synonymous and are intended to qualify the amount of each agent that will achieve the goal of improvement in disease severity and/or the frequency of incidence over treatment of each agent by itself while preferably avoiding or minimizing adverse side effects, including side effects typically associated with other therapies.

In some contexts, a “pharmaceutical carrier,” “diluent” or “excipient” includes, but is not limited to, any physiological buffered (i.e., about pH 7.0 to 7.4) medium comprising a suitable water soluble organic carrier, conventional solvents, dispersion media, fillers, carriers, coatings, antibacterial and antifungal agents, isotonic and absorption delaying agents.

In some contexts, the term “administering” and variations of that term including “administer” and “administration”, includes contacting, applying, delivering, or providing a compound or composition of the invention to a subject by any appropriate means.

### IV. Exemplary Embodiments

Among the provided embodiments are:

1. A method of treating fibromyalgia (FM), the method comprising administering to a subject suffering from one or more symptoms of FM, one or more doses of psilocybin or an active metabolite thereof effective to induce a dissociative state.

2. A method of treating fibromyalgia (FM), the method comprising orally administering to a subject suffering from one or more symptoms of FM, one or more doses of psilocybin or an active metabolite thereof effective to induce a dissociative state.

3. The method of embodiment 1 or 2, further comprising providing one or more integration sessions to the subject after emergence from the dissociative state.

4. The method of any of embodiments 1-3, further comprising, assessing in the subject one or more indicators associated with the one or more symptoms of FM, dissociative state, treatment outcome, safety and/or is related to the subject, prior to and/or after administration of the one or more doses of psilocybin.

5. A method of treating fibromyalgia (FM), the method comprising:
administering to a subject suffering from one or more symptoms of FM, one or more doses of psilocybin or an active metabolite thereof effective to induce a dissociative state;
providing one or more integration sessions to the subject after emergence from the dissociative state; and assessing in the subject one or more indicators associated with the one or more symptoms of FM, dissociative state, treatment outcome, safety and/or is related to the subject, prior to and/or after administration of the one or more doses of psilocybin or an active metabolite thereof.

6. A method of treating fibromyalgia (FM), the method comprising assessing in a subject suffering from one or more symptoms of FM, one or more indicators associated with the one or more symptoms of FM, dissociative state, treatment outcome, safety and/or is related to the subject, prior to and/or after administration of one or more doses of psilocybin or an active metabolite thereof effective to induce a dissociative state and one or more integration sessions after emergence from a dissociative state induced by the one or more doses of psilocybin or an active metabolite thereof.

7. A method of treating fibromyalgia (FM), the method comprising:
administering to a subject suffering from one or more symptoms of FM, one or more doses of psilocybin or an active metabolite thereof effective to induce a dissociative state; providing one or more integration sessions to the subject after emergence from the dissociative state; and
assessing in the subject one or more indicators prior to and/or after administration of the one or more doses of psilocybin or an active metabolite thereof; wherein the one or more indicators comprises one or more of: a fibromyalgia survey score, a pain interference assessment, a sleep assessment, a chronic pain acceptance assessment, or a clinical indicator assessment.

8. The method of any of embodiments 1-7, further comprising, prior to administering the one or more doses of psilocybin, assessing one or more of the following exclusion parameters in the subject:
(1) use of prior or concomitant medications;
(2) hypertension and/or tachycardia;
(3) incidence of epilepsy;
(4) incidence of schizophrenia spectrum or other psychotic disorders;
(5) a moderate or severe alcohol or drug use disorder; and
(6) prior adverse effects from psilocybin.

9. The method of embodiment 8, further comprising identifying the subject for treatment with psilocybin if the subject does not meet any of the exclusion parameters.

10. A method of identifying a subject for treating fibromyalgia (FM) with psilocybin or an active metabolite thereof, the method comprising:
(a) assessing in a subject suffering from one or more symptoms of FM, one or more of the following exclusion parameters:
   (1) use of prior or concomitant medications;
   (2) hypertension and/or tachycardia;
   (3) incidence of epilepsy;
   (4) incidence of schizophrenia spectrum or other psychotic disorders;
   (5) a moderate or severe alcohol or drug use disorder; and
   (6) prior adverse effects from psilocybin or an active metabolite thereof.
(b) identifying the subject for treatment with one or more doses of psilocybin or an active metabolite thereof if the subject does not meet any of the exclusion parameters.

11. A method of identifying a subject for treating fibromyalgia (FM) with one or more doses of psilocybin or an active metabolite thereof, the method comprising identifying a subject suffering from one or more symptoms of FM for treatment if the subject does not meet any of the following exclusion parameters that are assessed in the subject:
(1) use of prior or concomitant medications;
(2) hypertension and/or tachycardia;
(3) incidence of epilepsy;
(4) incidence of schizophrenia spectrum or other psychotic disorders;
(5) a moderate or severe alcohol or drug use disorder; and
(6) prior adverse effects from psilocybin or an active metabolite thereof.

12. The method of embodiment 10 or 11, further comprising:
administering to the identified subject, one or more doses of psilocybin or an active metabolite thereof effective to induce a dissociative state; and
providing one or more integration sessions to the subject after emergence from the dissociative state.

13. The method of any of embodiments 10-12, further comprising assessing in the subject one or more indicators associated with the one or more symptoms of FM, dissociative state, treatment outcome, safety and/or is related to the subject, prior to and/or after administration of the one or more doses of psilocybin or an active metabolite thereof.

14. The method of any of embodiments 1-13, wherein the administering is by oral administration or intravenous administration.

15. The method of any of embodiments 1-14, wherein the administering is by oral administration.

16. The method of any of embodiments 1-14, wherein the psilocybin or an active metabolite thereof is psilocybin.

17. The method of any of embodiments 4-9 and 13-16, wherein the one or more indicators comprise observer-rated parameters, subject-reported parameters and/or clinical indicators.

18. The method of any of embodiments 4-9 and 13-17, wherein the one or more indicators are assessed prior to the administration as a baseline measurement, and after the administration as an outcome measurement, and the changes between the baseline measurement and the outcome measurement are determined.

19. The method of any of embodiments 4-9 and 13-18, wherein the one or more indicators comprises one or more of: a fibromyalgia survey score, a pain interference assessment, a sleep assessment, a chronic pain acceptance assessment, or a clinical indicator assessment.

20. The method of any of embodiments 4-9 and 13-19, wherein the one or more indicators comprises one or more of: a fibromyalgia survey score, optionally a 2016 fibromyalgia survey questionnaire (FSQ) assessment; a pain intensity numerical rating scale (PI-NRS) assessment, optionally an 11-item PI-NRS scale; a PROMIS pain interference assessment; a PainDETECT Questionnaire (PD-Q) assessment; a chronic pain acceptance assessment, optionally measured by chronic pain acceptance questionnaire (CPAQ-8) assessment; a pain interference assessment, optionally measured by 4-item PROMIS pain interference scale; and a Complex Medical Symptom Inventory (CMSI).

21. The method of any of embodiments 4-9 and 13-20, wherein the one or more indicators comprises a fibromyalgia survey score.

22. The method of any of embodiments 7 and 19-21, wherein the fibromyalgia survey score comprises a 2016 fibromyalgia survey questionnaire (FSQ) assessment.

23. The method of any of embodiments 4-9 and 13-22, wherein the one or more indicators comprises a pain intensity numeric rating scale (PI-NRS) 11-item pain scale.

24. The method of any of embodiments 4-9 and 13-23, wherein the one or more indicators comprises one or more of: a sleep impairment assessment, optionally measured by a PROMIS sleep-related impairment 4a assessment; a PROMIS sleep disturbance 8b assessment; and a STOP-Bang questionnaire.

25. The method of any of embodiments 4-9 and 13-24, wherein the one or more indicators comprises one or more of: a PROMIS-29+2 Profile, optionally a v2.1 multi-domain assessment; a positive and negative affect score (PANAS) assessment; a patient global impression of change (PGI-C) scale assessment; and a patient acceptable symptom state (PASS) questionnaire.

26. The method of any of embodiments 4-9 and 13-25, wherein the one or more indicators comprises a PROMIS-29+2 Profile v2.1 multi-domain assessment.

27. The method of any of embodiments 4-9 and 13-26, wherein the one or more indicators comprises one or more of: a Patient Health Questionnaire depression scale-8 (PHQ-8); a Columbia Suicide Severity Rating Scale (C-SSRS); a Clinician Global Impression—Improvement (CGI-I) scale; an Emotional Breakthrough Inventory (EBI); a Acceptance and Action Questionnaire-II (AAQ-II); a Patient Global Impression-Improvement (PGI-I); a Hospital Anxiety and Depression Scale (HADS); and a Life Events Checklist (LEC).

28. The method of any of embodiments 4-9 and 13-27, wherein the one or more indicators comprises a Patient Health Questionnaire depression scale-8 (PHQ-8).

29. The method of any of embodiments 4-9 and 13-28, wherein the one or more indicators comprises one or more of: subject-reported mystical experiences assessment, optionally measured by a Mystical Experience Questionnaire (MEQ30); a subject-reported challenging experiences assessment, optionally measured by a Challenging Experience Questionnaire (CEQ27); a subject-reported psychological insight assessment, optionally measured by the Psychological Insight Questionnaire (PIQ); and a subject-reported qualitative written assessment.

30. The method of any of embodiments 4-9 and 13-29, wherein the one or more indicators comprises therapist-reported monitor rating scale, optionally measured using a Monitor Rating Scale (MRS) form.

31. The method of any of embodiments 4-9 and 13-30, wherein the one or more indicators comprises a Quantitative Sensory Testing (QST).

32. The method of embodiment 31, wherein a QST is measured using Cuff Pressure Pain assessment, optionally cuff-PPT, cuff-Pain40, cuff-Pain50, and/or cuff-tolerance.

33. The method of any of embodiments 4-9 and 13-31, wherein the one or more indicators comprises a Visual Hypersensitivity Assessment.

34. The method of any of embodiments 4-9 and 13-33, wherein the one or more indicators comprises an electroencephalogram (EEG).

35. The method of any of embodiments 4-9 and 13-33, wherein the one or more indicators comprises QST and EEG, and the QST and the EEG are performed concurrently.

36. The method of any of embodiments 4-9 and 13-35, wherein the one or more indicators comprises functional magnetic resonance imaging (fMRI).

37. The method of embodiment 36, wherein the fMRI comprises one or more of: structural MRI; resting fMRI; proton magnetic resonance spectroscopy (1H-MRS) in the right anterior insula; fMRI with tonic cuff pressure evoked experimental pain; fMRI with block design cuff pressure pain; and diffusion tensor imaging (DTI).

38. The method of any of embodiments 4-9 and 13-37, wherein the one or more indicators comprises one or more of: wrist actigraphy, optionally wherein wrist actigraphy is used to measure sleep onset latency; wake after sleep onset; total sleep time; and sleep efficiency (%).

39. The method of any of embodiments 4-9 and 13-38, wherein the one or more indicators comprises an electrocardiograms (ECG).

40. The method of any of embodiments 4-9 and 13-38, wherein the one or more indicators are measured during one or more of the integration sessions.

41. The method of any of embodiments 8-40, wherein the exclusion parameter is the use of prior or concomitant medications, and the subject is identified for treatment if the subject is not using any of the following prior or concomitant medications:
regular use of psychoactive prescription medication, opioids, tramadol, or benzodiazepines; concomitant use of antidepressants; regular use of medications having a primary centrally-acting serotonergic effect or monoamine oxidase inhibitors (MAOIs); use of hallucinogens in the past 6 months; concomitant use of sildenafil or tadalafil; concomitant use of alcohol with greater than 0.02% blood alcohol content; antihypertensive medications; UGT1A9 inhibitors, 1A10 inhibitors, regorafenib, rimampicin, phenytoin, eltrombopag, mefenamic acid, diflunisal, niflumic acid, sorafenib, isavuconazole, deferasiroxor, ginseng; aldehyde dehydrogenase inhibitors, alcohol dehydrogenase inhibitors, or disulfiram; amphetamines, buprenorphine, benzodiazepines, cocaine, methamphetamines, Ecstasy (MDMA), morphine, methadone, oxycodone, marijuana, ethyl glucuronide, fentanyl, tramadol, synthetic cannabinoids (K2) regular use of psychoactive prescription medication, opioids, tramadol, or benzodiazepines; concomitant use of antidepressants; regular use of medications having a primary centrally-acting serotonergic effect, monoamine oxidase inhibitors (MAOIs), or selective serotonin reuptake inhibitors (SSRIs), or use of serotonin-acting dietary supplements, 5-hydroxy-tryptophan, or St. John's wort,

42. The method of any of embodiments 8-41, wherein the exclusion parameter is hypertension, and the subject is identified for treatment if the subject does not exhibit a systolic blood pressure of greater than at or about 139 mm Hg, a diastolic blood pressure of greater than at or about 89 mm Hg; and/or wherein the exclusion parameter is tachycardia and the subject is identified for treatment if the subject does not exhibit a heart rate of greater than at or about 90 beats per minute (bpm); optionally when measured as an average of three readings.

43. The method of any of embodiments 6-42, wherein the exclusion parameter is incidence of epilepsy and the subject is identified for treatment if the subject does not have epilepsy.

44. The method of any of embodiments 6-43, wherein the exclusion parameter is incidence of psychotic disorders and the subject is identified for treatment if the subject does not have a schizophrenia spectrum or other psychotic disorder that meets the DSM-5 criteria, major depressive disorder with psychotic features, or Bipolar I or Bipolar II Disorder.

45. The method of any of embodiments 6-44, wherein the exclusion parameter is a moderate or severe alcohol or drug use disorder, and the subject is identified for treatment if the subject does not have a moderate or severe alcohol or drug use disorder that meets the DSM-5 criteria.

46. The method of any of embodiments 6-45, wherein the exclusion parameter is prior adverse effects from psilocybin or an active metabolite thereof, and the subject is identified for treatment if the subject has not had a prior adverse effects from psilocybin or an active metabolite thereof.

47. The method of any of embodiments 4-9 and 13-46, wherein the subject exhibits amelioration of the one or more indicators between the baseline measurement and the outcome measurement.

48. The method of any one of the embodiments 1-47, wherein the subject receiving treatment shows improvement in one or more symptoms of FM or one or more symptoms of FM are ameliorated.

49. The method of any one of the embodiments 1-48, wherein the subject receiving treatment shows improvements selected from among one or more of: a fibromyalgia survey score, a pain interference assessment, a sleep assessment, a chronic pain acceptance assessment, or a clinical indicator assessment.

50. The method of any one of the embodiments 1-49, wherein the subject receiving treatment shows improvements selected from among one or more of: a fibromyalgia survey score, optionally a 2016 fibromyalgia survey questionnaire (FSQ) assessment; a pain intensity numerical rating scale (PI-NRS) assessment, optionally an 11-item PI-NRS scale; a PROMIS pain interference assessment; a PainDETECT Questionnaire (PD-Q) assessment; a chronic pain acceptance assessment, optionally measured by chronic pain acceptance questionnaire (CPAQ-8) assessment; and a pain interference assessment, optionally measured by 4-item PROMIS pain interference scale.

51. The method of any of embodiments 1-50, wherein the one or more doses of psilocybin or an active metabolite thereof comprises a first dose of psilocybin.

52. The method of embodiment 51, wherein the first dose is between at or about 10 mg to at or about 50 mg of psilocybin.

53. The method of embodiment 51 or 52, wherein the first dose is at or about 15 mg of psilocybin.

54. The method of any of embodiments 1-53, wherein the one or more doses of psilocybin comprises a two or more doses of psilocybin.

55. The method of any of embodiments 1-54, wherein the one or more doses of psilocybin comprises a second dose of psilocybin.

56. The method of embodiment 55, wherein the second dose is between at or about 20 mg to at or about 50 mg of psilocybin.

57. The method of embodiment 55 or 56, wherein the second dose is at or about 25 mg of psilocybin.

58. The method of embodiment 55, wherein the second dose is between at or about 10 mg to at or about 20 mg of psilocybin

59. The method of embodiment 58 or 59, wherein the second dose is at or about 15 mg of psilocybin.

60. The method of any of embodiments 55-59, wherein the second dose is administered between at or about 12 days and at or about 16 days after the first dose.

61. The method of any of embodiments 55-60, wherein the second dose is administered at or about 14 days after the first dose.

62. The method of any of embodiments 1-61, further comprising providing one or more integration sessions to the subject after the one or more doses of psilocybin.

63. The method of embodiment 62, wherein the one or more integration sessions is provided at or about 3, 4, and/or 5 weeks after administering the first dose of psilocybin.

64. The method of embodiment 62 or 63, wherein the one or more integration sessions is provided at or about 1, 2, and/or 3 weeks after administering the second dose of psilocybin.

### V. Examples

The following examples are included for illustrative purposes only and are not intended to limit the scope of the invention.

### Example 1: Effect of Intravenous Administration of Psychedelics on Diseases Associated with Pain

This example describes the effects of intravenous administration of psychedelics on pain perception and diseases associated with pain, such as nociplastic pain, in an animal model of pain. A rat model of formalin (FML)-induced chronic pain was used to investigate the effect of psilocybin administration on mechanical allodynia and thermal hyperalgesia.

Chronic pain in rats was induced by injection of formalin into the hind paw. The rats subsequently were intravenously administered psilocybin, and assessed for changes in pain perception over an extended period of time. The formalin pain model is a translational animal model for chronic nociplastic pain (Vanini G., Sleep 2016; 39(1):133-42), including fibromyalgia (FM). In the formalin pain model, in addition to experiencing extra pain in the formalin-injected paw, the animal experiences increased pain perception (i.e., hyperalgesia) in extremities not injected with formalin (e.g., the contralateral hind paw) compared to the one injected with formalin (e.g., the ipsilateral hind paw). The pain perception was measured in the contralateral and ipsilateral paws to assess the potential benefit of psychedelics (e.g., psilocybin) in nociplastic pain disorders. Reduction in pain perception and the duration of pain reduction were examined following intravenous bolus administration of 10 mg/kg psilocybin or saline, each administered over 3 minutes.

Adult male Sprague Dawley rats (300-350 g) were implanted with a catheter in the jugular vein for delivery of 10 mg/kg psilocybin or 0.9% saline. Mechanical allodynia was assessed using von Frey assay (VFA) and was expressed as the threshold in grams (g). Thermal hyperalgesia was measured as paw withdrawal latency in seconds (s) using the hot plate assay (HPA) (52.5° C.). After post-surgical recovery (7-10 days), baseline responses to mechanical and thermal stimuli were measured. 24 h after baseline measurements, rats received a subcutaneous formalin injection (5%, 50 μL) into one of the hind paws (Day 0). Two hours after the formalin injection, paw withdrawal thresholds in response to mechanical and thermal stimuli were measured. 24 h after FML injection (Day 1), rats received intravenous infusion of 10 mg/kg psilocybin (N=6) or 0.9% saline (N=6), and 3-4 h after the injections, paw withdrawal thresholds in VFA and HPA were measured. Rats were tested every other day during the first week, and then weekly for a total of 4 weeks. The data are reported as mean±standard deviation.

Two types of pain perception were measured. First, the Von Frey assay (VFA) was used to measure pain due to pressure exerted on the extremity (i.e., mechanical allodynia). Specifically, VFA employed seven filaments: 1, 2, 4, 6, 8, 10 and 15 g. Each filament was applied in ascending order to the plantar surface of the rat's hind paws (contralateral and ipsilateral) until bent. Upon bending the filament, the filament was held in place for 5 seconds or until the rat withdrew its paw. The stimulus was not applied if the rat was walking, grooming, or sniffing. The stimulus was applied to the injected, ipsilateral and contralateral hind paws for five trials each, with at least 30 second breaks between stimuli. The minimum amount of force required to evoke a paw withdrawal response in each trial was determined to be the minimum withdrawal threshold for that animal trial. After five trials for each paw, the minimum amount of force required to evoke a paw withdrawal response was averaged across all trials to calculate the average minimum withdrawal threshold for each paw, respectively.

The second pain measurement was based on the Hot Plate Assay (HPA), which measures the withdrawal latency at temperatures that register a painful response (i.e., thermal hyperalgesia). The HPA was initiated 15-20 minutes after completion of the VFA. A hot plate was preheated to a temperature of 52.5° C. Once heated, each animal was placed in cylindrical tube on the hot plate and the latency to lick a hind paw or observed overt escape behavior (e.g., attempt to jump out of cylinder) was recorded (Ingram et al., Neruopsychopharmacology 2007; 32(3):600-6; Gunn et al., J Pain 2011; 12(2):222-7; Hestehave et al., Lab Anim 2016; 51(3):264-72). For safety purposes, the maximum time for a rat to be on hot plate was limited to 30 seconds. After a 15 minute break, this was exercise was repeated once more.

The full examination of pain perception lasted for 28 days for each animal, as described Table E1 below.

TABLE E1
Testing Procedure and Schedule

| Day(s) | Procedures | |
|---|---|---|
| Day −1 | I. | Test VFA withdrawal threshold baseline at 2 PM |
| (baseline) | | (+/−1 hour) |
| | II. | If baseline VFA showed abnormal results (<10 g), |
| | | the animal was reevaluated. Rats that show |
| | | persistent, reduced baseline levels (<10 g) were |
| | | excluded from the study |
| | III. | Test HPA withdrawal latency baseline at 3 PM |
| | | (+/−1 hour) |
| Day 0 | I. | Administer subcutaneous 50 μl injection of 5% |
| (formalin | | formalin on the ventral surface of right hind paw |
| baseline) | | (around 12 PM). Right and left hind paw were |
| | | counterbalanced for each experimental cohort (i.e., |
| | | some rats received formalin injection in the left paw, |
| | | others in the right paw) |
| | II. | Measure VFA withdrawal threshold (1-3 PM) |
| | III. | Measure HPA withdrawal latency (2-4 PM) |
| Day 1 | I. | Administer 10 mg/kg intravenous psilocybin in a |
| | | 0.9% saline, or saline only control, over 3 minutes |
| | | (11 AM) |
| | II. | Measure VFA withdrawal threshold (1-3 PM) |
| | III. | Measure HPA withdrawal latency (2-4 PM) |
| Days 3, 5, 7, | I. | Measure VFA withdrawal threshold (1-3 PM) |
| 14, 21, and 28 | II. | Measure HPA withdrawal latency (2-4 PM) |

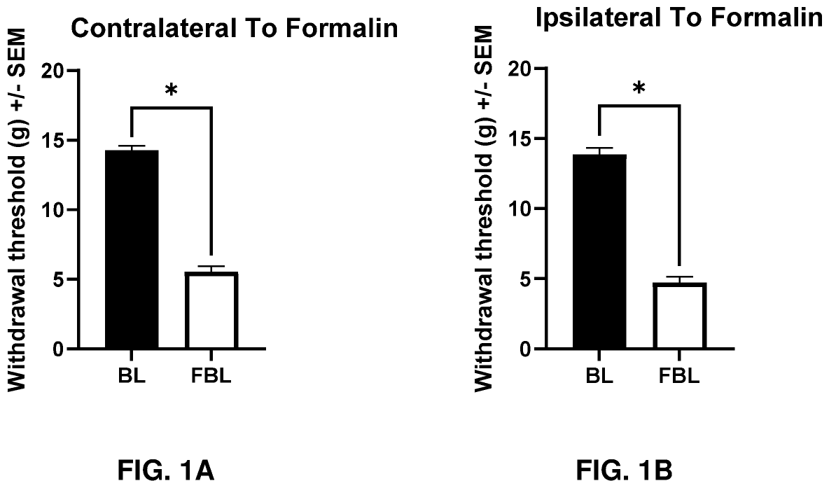

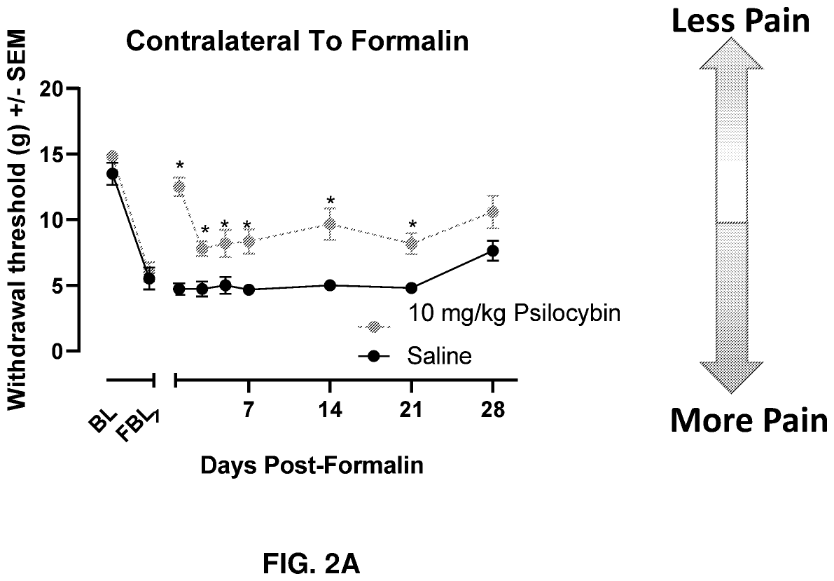

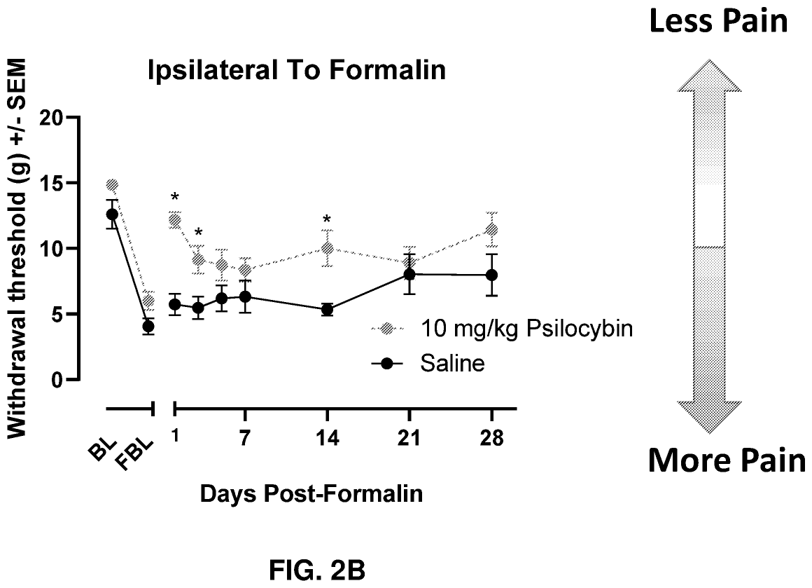

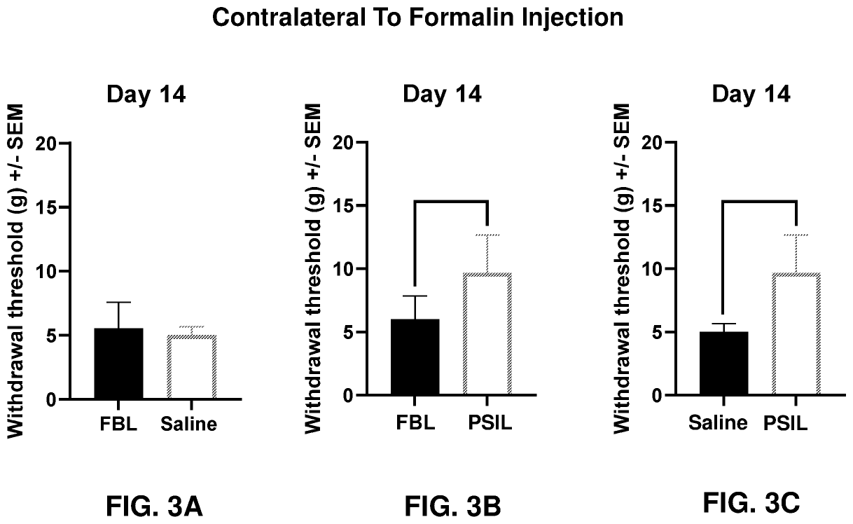

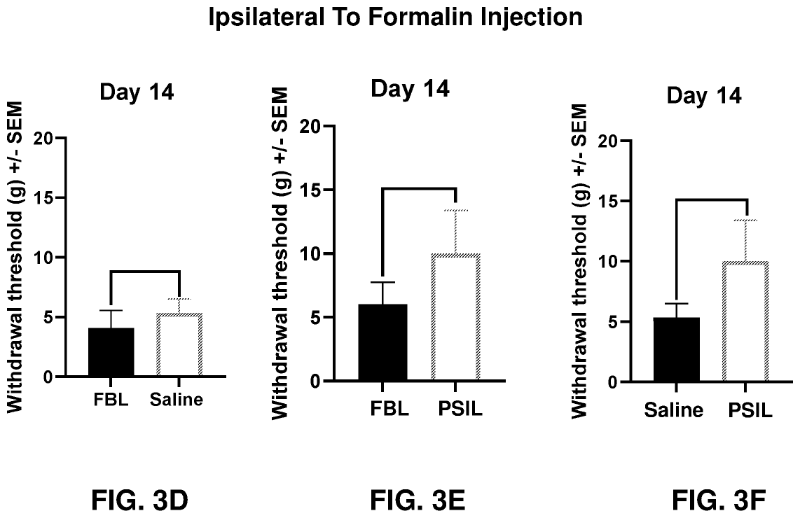

FIG. 1A and FIG. 1B show the results for the VFA, comparing the effects of formalin injection (day 0) on withdrawal threshold (i.e., mechanical allodynia) for the contralateral (FIG. 1A) and ipsilateral (FIG. 1B) paws. The withdrawal threshold for both groups was initially high at baseline (BL) and substantially reduced after formalin injection (FBL). After psilocybin or saline injection on day 1, a marked and acute difference in withdrawal threshold was observed for psilocybin-injected rats in both the contralateral paw (FIG. 2A) and the ipsilateral paw (FIG. 2B), indicative of analgesic properties of psilocybin administration. For other analgesic drugs (e.g., NSAID, opioids), this response rapidly disappears after drug clearance from the circulation. In contrast, rats administered intravenous psilocybin showed reduced pain sensitivity for at least two weeks after a single psilocybin administration, for both the contralateral (FIGS. 3A-3C) and the ipsilateral paw (FIGS. 3D-3F), demonstrating a sustained analgesic effect. In the contralateral paw, a statistically significant increase in withdrawal threshold was observed for 3 weeks after a single psilocybin administration (FIG. 2A).

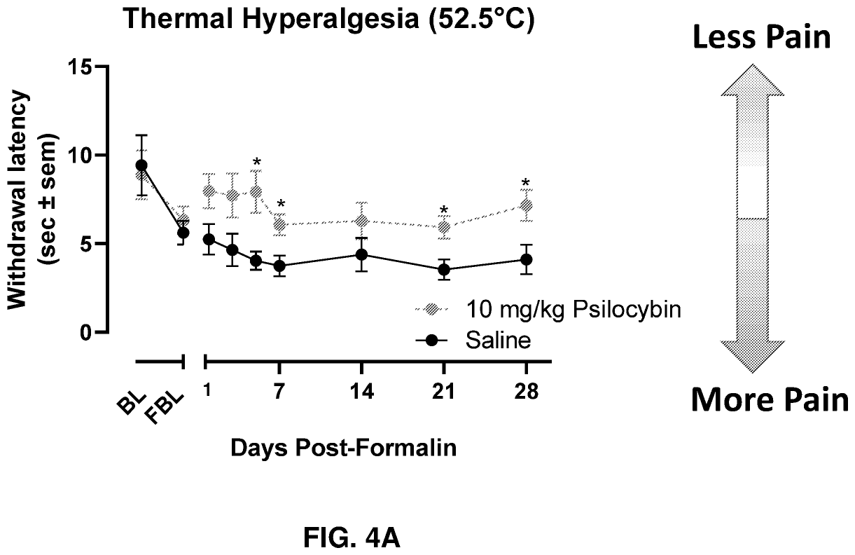

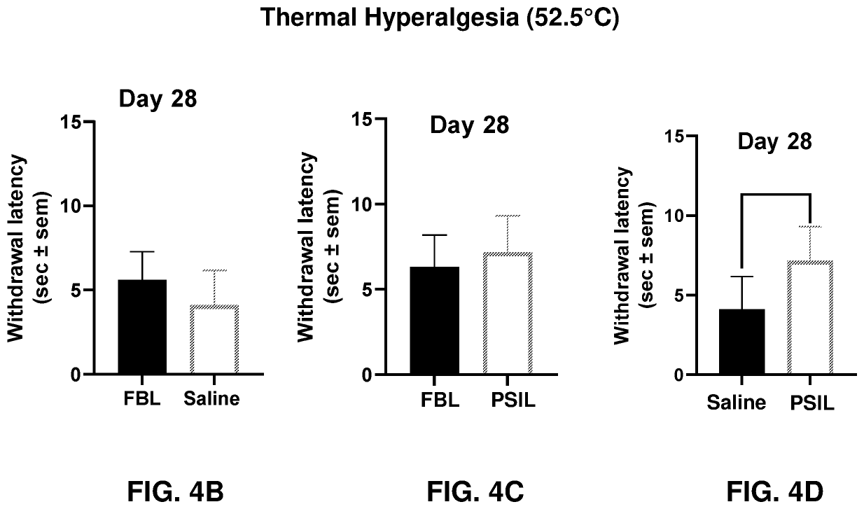

As shown in FIG. 4A, similar results were observed for thermal sensitivity, as shown by the HPA results. After an acute recovery response in withdrawal latency after psilocybin administration, the improved pain sensitivity response was sustained for at least 4 weeks compared to saline control (FIGS. 4B-4D), demonstrating a sustained analgesic effect.

The results demonstrated acute and lasting effects of intravenous administration of psychedelics, such as psilocybin, on mechanical and thermal pain sensitivity. The results showed long-lasting improvements on pain sensitivity, for at least up to 4 weeks in an animal model of nociplastic pain following a single administration of a psychedelic, such as psilocybin. The effects were observed for the non-injected contralateral paws, which is closely associated with nociplastic pain. The results support the utility of administering psychedelics for treating and improving diseases and disorders related to pain perception, including nociplastic pain disorders such as fibromyalgia (FM).

### Example 2: Administration of Psilocybin and Psychotherapy in Subjects with Fibromyalgia

Current fibromyalgia (FM) management strategies and medications provide only modest benefit and impose a substantial side effect burden, indicating a need for therapies with improved effect and safety. The therapeutic effect and safety of two separate oral doses of psilocybin in combination with psychotherapy is assessed in a clinical study of human subjects with FM.

### A. Inclusion Criteria

Inclusion criteria related to FM disease characteristics include but are not limited to a diagnosis of FM for at least or for longer than 3 months, having experienced FM symptoms for at least 1 year, or a score of ≥13 on the 2016 FM survey criteria for at least or over 1 year. Subjects are eligible to be included in the study only if all inclusion criteria are met.

### B. Exclusion Criteria

Prospective subjects are excluded from the study based on certain criteria, including but not limited to, physiological and psychological medical conditions, certain prior and/or concomitant therapies, and diagnostic assessments made during subject screening. A prospective subject is excluded if any of the exclusion criteria apply.

### C. Study Design

Subjects that have FM or have had FM symptoms for at least a year, and meet the 2016 FM Survey Criteria for FM are included in a clinical study.

The total duration of study participation for an individual is approximately 8 months (FIG. 5). The study comprises a screening period of up to 28 days prior to initiation of the study (Visit 1; Days −28 to −1), a baseline assessment (Visit 2; Days 1-7), a one-week preparation period comprising two therapy sessions (Visits 3 and 4; Day 11 and 18), and a dosing period comprising follow-up sessions (Visits 5 to 9; Days 22 to 37), wherein two doses of psilocybin, separated by two weeks, are administered to a subject. A two-week integration period begins after the final dose of psilocybin is administered (Visits 10 to 12; Days 43 to 57), and end of the therapy (EOT) (Visit 13; Day 64) assessments are made one week after the final integration session. Follow-up evaluations are made at three and six months after the second, final dose of psilocybin. Flexibility in scheduling of at least ±3 days is allotted for each visit, beginning at initiation of preparation therapy, Visit 3.

Patient reported outcomes are assessed from Day 0 to the final 6-month follow-up visit. The end of the study is defined as the date of the last visit of the last study subject. A subject is considered to have completed the study if all phases of the study, including the 6-month follow-up visit, are completed. Flexibility in scheduling study visits and sessions is allowed to account for unexpected absences, as there is no reason to believe that minor differences in the timing of the study visits and measures to have an adverse effect on subjects or on the validity of the study.

### 1. Screening (Visit 1; Days −28 to −1)

Candidates who express interest in participating in the study are pre-screened, either electronically or over the phone. Potential subjects are interviewed to determine whether inclusion/exclusion criteria are met. Potential subjects are invited for an in-person screening visit. The informed consent process may occur either electronically prior to the scheduled screening visit or during the screening visit prior to performance of any assessments.

Once candidates are pre-screened for interest and all eligibility pre-screening measures have been met, subjects complete a screening visit (Visit 1). Inclusion and exclusion criteria are confirmed. The Structured Clinical Interview for DSM-5 (SCID-5) and SCID-5-PD (for Personality Disorders) are administered to determine whether a subject meets the criteria for certain mental disorders, for example, schizophrenia, other psychotic disorders, bipolar I or II disorder, or severe alcohol, tobacco, or other drug use disorder, which would lead to exclusion from the study. Demographic information and medical history is also assessed.

Prior and concomitant medications are evaluated at this visit and at each subsequent visit. Physical and physiological characteristics are assessed at this initial visit and throughout the study, including, for example, 12-lead electrocardiogram (ECG), pregnancy test for women of childbearing potential or a follicle stimulating test (FSH) in postmenopausal women, urine drug and breath alcohol tests, and vital signs. Subject-reported outcomes are collected, including, for example, responses to the 2016 Fibromyalgia (FM) Survey Criteria, Patient Health Questionnaire depression scale (PHQ-8), and the Columbia Suicide Severity Rating Scale (C-SSRS). Responses to the C-SSRS are evaluated at each visit through the 6-month follow up, as new onset of suicidal ideation is considered an adverse event (AE).

### 2. Baseline Pain Phenotyping (Visit 2; Days 1 to 7)

During the second study visit, subjects undergo a rigorous pain phenotyping assessment, referred to herein as deep phenotyping which serves as a baseline assessment for comparison with subsequent evaluations. AEs are evaluated at each visit through the end of active treatment (End of Therapy; Visit 13), when a final deep phenotyping assessment is completed, and are reported through applicable channels. Deep phenotyping includes a validated battery of subject-reported outcomes related to pain and quality of life. Self-reporting surveys include, for example, the Numerical Rating Scale (NRS) Daily Pain Diary (0-10), Sleep-related disturbance 8b, Sleep-related Impairment (4a), painDETECT, the catastrophizing scale from the Coping Strategies Questionnaires Pain, Chronic Pain Acceptance Questionnaire 8 (CPAQ-8), C-SSRS, Snoring, Tiredness, Observed apnea, blood Pressure, Body mass index, Age, Neck circumference and Gender (STOP-Bang), 2016 FM Survey Criteria, Positive and Negative Affect Score (PANAS) short form (state), Life Events Checklist, Complex Multi-symptom Inventory (CMSI), and Patient Reported Outcomes Measurement Information System 29-item profile measure (PROMIS-29+2).

From this initial deep phenotyping session until the End of Therapy (EOT) visit, subjects complete a daily electronic pain diary, recording their pain level on a scale of 0 to 10 on the Pain Intensity-Numeric Rating Scale (PI-NRS). Subjects receive a text or email every evening with the pain diary questions. If the subject does not respond within two hours, a reminder text or email is sent. Response options are closed by midnight. Subjects who miss a day are contacted the following day to remind them to complete their daily diary. Self-reported NRS in the Daily Pain Diary is evaluated at each visit through the EOT visit, during which a final deep phenotyping assessment is completed.

Neurological and other physiological assessments are also performed during deep phenotyping. Neurological assessments include, for example, functional MRI (fMRI) and electroencephalography (EEG). Quantitative sensory testing (QST) is performed, which includes, for example, tasks related to pain pressure thresholds and pain catastrophizing, the tendency to magnify the threat value of a pain stimulus and to feel helpless in the experience. A means for wrist actigraphy, a non-invasive method of monitoring cycles of rest and activity, is provided to the subject, and actigraphy measurements are evaluated at each visit through EOT. Vital signs and an inquiry into prior and concomitant medications are determined at baseline and throughout the study.

### 3. Preparatory Sessions (Visits 3 and 4; Days 11 to 18 (±3 Days)

Subjects begin the psilocybin-psychotherapy intervention by preparing for psilocybin administration. Subjects undergo eight hours of preparatory therapy in weeks 1 and 2 (four hours per preparatory session). During these preparatory visits, subjects develop rapport with two designated therapist facilitators, gain understanding of what will occur during dosing sessions, and build comfort and understanding around the physical space in which the psilocybin session takes place.

### 4. Psilocybin Administration Sessions (Visits 5 to 9; Days 22 to 37 (±3 Days)

Subjects receive two doses of psilocybin (15 mg and 25 mg) separated by the span of two weeks. Psilocybin sessions, which last approximately eight hours, are held under the supervision of two therapist facilitators. During the psilocybin administration sessions, subjects orally ingest psilocybin capsule(s), a fixed dose of 15 mg during the first session or a 25 mg dose during the second session. The fixed 25 mg dose of psilocybin approximates the circulating levels of psilocin that occur when psilocybin is administered at a 0.3 mg/kg dose (Brown et al., Clin Pharmacokinet. 2017; 56(12):1543-1554 and Dahmane et al. Clin Pharmacol Drug Dev. 2021; 10(1):78-85). If the subject or the study investigator determines that the higher dose may not provide further benefit, the 15 mg dose can be repeated during the second session.

Sessions are conducted in a room designed to be quiet, comfortable, and aesthetically pleasing. Subjects are encouraged to wear eyeshades and listen to a program of music through headphones during the drug exposure to aid them in focusing their attention inward. Audio and video of each session is recorded. After the effects of psilocybin have subsided, subjects complete the Mystical experiences Q-30, Challenging experiences Q, and Psychological Insight Q questionnaires. The PROMIS-29+2 is administered at each dosing session. Patients complete the Patient Global Impression of Change (PGI-C) questionnaire after the first dose of psilocybin and at each subsequent visit through the 6-month follow-up.

To ensure subject safety and comfort, two follow-up visits are held after the first dosing session (one day and one week after administration). An additional follow-up session is held one day after the second administration of psilocybin. Participants are asked to process the experience at home by writing a reflection about their experiences during the dosing session. This reflection is discussed during the follow-up meetings.

### 5. Integration Period (Visits 10 to 12; Days 43 to 57 (±3 Days)

One week after the second and final dosing session, subjects complete three one-hour long integration sessions over the course of three weeks. To promote integration, psychotherapy is conducted with a focus on helping subjects gain insight and understanding from their experiences. Sleep, by means of wrist actigraphy, and responses to the 2016 FM Survey Criteria and PGI-C are assessed during the integration period.

### 6. End of Therapy (Visit 13; Day 64 (±3 Days)

Subjects complete their final deep phenotyping session during the EOT visit, which occurs approximately 4 weeks after the final dose of psilocybin. Assessments include those made during the initial deep phenotyping session, which are important for evaluation of primary, secondary, and exploratory endpoints. Subject documented NRS are collected from the electronic diary, vital signs are taken, and concomitant medications are reported. Any AEs are documented and reported through the appropriate applicable channels.

### 7. Follow-Up Period (Visits 14 and 15; Day 120 and Day 204 (±7 Days)

Subjects complete electronic subject-reported outcomes questionnaires at three months and six months after the final psilocybin dose to examine persistence of changes in primary, secondary, and exploratory endpoints. Assessments include the C-SSRS, 2016 FM survey criteria, PANAS) short form, PGI-C, Sleep-related disturbance 8b, Sleep-related Impairment (4a), CPAQ-8, painDETECT, PANAS short form (state), Catastrophizing, Psychological Insight Q, and PROMIS-29+2. Neurological assessments are also performed during these visits, including fMRI, EEG, and QST. A qualitative written assessment is completed at the 3-month follow-up visit to determine subjective experiences.

### D. Study Endpoints

An endpoint of the study is to evaluate the safety of oral psilocybin in combination with psychotherapy to treat chronic pain symptoms in subjects with FM. Safety assessments are always performed before efficacy assessments at each visit. Safety is assessed during psilocybin dosing sessions and throughout the trial.

Safety is evaluated during the dosing sessions by measuring a subject's vital signs, for example blood pressure (BP), pulse, and heart rate (HR), as well as the dissociative state. Vital signs, for example, BP and HR, re measured before psilocybin administration and at 30, 60, 90, 120, 180, 240, 300, and 360 minutes after administration. BP and pulse measurements are preceded by at least 5 minutes of rest for the subject in a quiet setting without distractions. BP and pulse measurements are assessed with a completely automated device. Manual techniques are used only if an automated device is not available At the same time points at which the BP and HR are measured, the session monitors complete a Monitor Rating Form, which involves rating or scoring several dimensions of the subject's behavior and mood, e.g., presence/intensity of behaviors, signs, and reported symptoms, such as peacefulness, yawning, nausea/vomiting, quantity of speech, anxiety, sleepiness, crying, restlessness, visual changes, euphoria, and feelings of unreality.

Safety throughout the trial is assessed by monitoring AEs, ECG measurements, and suicidality. All AEs from the time of signing of the informed consent form until EOT (Day 64) are collected and reported. Open-ended and non-leading verbal questioning of the subject is the preferred method to inquire about AE occurrences. ECG and pregnancy testing at baseline are performed to determine eligibility. Physical examinations and laboratory testing, including urine drug and breath alcohol testing are performed throughout the study. Pregnancy testing (for any woman or adolescent who has begun menstruation) is performed throughout the study to determine continued eligibility.

The C-SSRS is used to assess the severity of suicidal ideation during every in-person visit. The C-SSRS is used to assess severity and intensity of suicidal ideation, types of suicidal behaviors, and lethality of suicide attempts at set time points and over time periods that are typical for randomized controlled trials. Developed by researchers at Columbia University, it is widely used in clinical and research settings.

Other endpoints include assessing the clinical utility or efficacy of oral psilocybin in combination with psychotherapy. Efficacy endpoints include change in aggregate worst pain score, pain interference, sleep, chronic pain acceptance (CPAQ responses), and Patient Global Impression of Change (PGI-C responses). Pain score, pain interference, sleep disturbance, chronic pain acceptance, and the Patient Global Impression of Change were chosen as secondary measures given their relevance to FM symptom management (Clauw, JAMA. 2014; 311(15):1547-1555; Williams, Arthritis Care Res. 2011; 63 Suppl 11:S86-S97. doi:10.1002/acr.20531; Kratz et al., Ann Behav Med. 2007; 33(3):291-301; Farrar et al., Pain. 20001; 94(2):149-158).

Change in Aggregate Worst Pain Score: As described herein, self-reported assessments of pain are recorded throughout the study into daily electronic pain diaries. Baseline aggregate worst pain scores are compared to aggregate worst pain scores determined during the week prior to the EOT visit (Days 57 to 63). Therapeutic benefit is based on a comparison of the baseline score to the final score (NRS of 0 to 10). A 30% or 2-point decrease in aggregated worst pain scores at either movement or rest is considered a clinically significant response (Farrar et al. Pain.

2001; 94(2):149-158).

Pain Interference: In addition to the potential therapeutic benefit on chronic pain, the effects of psilocybin and psychotherapy on pain interference and chronic pain acceptance are also assessed. Pain interference is the degree to which pain affects important aspects of an individual's life, such as social, cognitive, and physical activities. Pain interference is assessed using the 4-item PROMIS pain interference scale from the PROMIS-29+2 Profile v2.1 (PROPr), which is available on HealthMeasures.net.

Sleep: Sleep and improvement or decline in clinical status are also evaluated. Sleep disturbances are measured using the PROMIS sleep disturbance 8b short form. This measure includes assessments of sleep quality, perceived ability to fall and stay asleep, satisfaction of sleep, and depth of sleep. This measure has some overlap with the PROMIS 29+2 PROPr, and items are consolidated accordingly.

Chronic Pain Acceptance: The 8-item Chronic Pain Acceptance Questionnaire (CPAQ-8) is a validated measure that assesses activity engagement and pain willingness (e.g., recognizing that trying to avoid or control pain may be maladaptive for chronic pain) (Fish et al., Pain.

2010; 149(3):435-443). Subjects rate items on a 0 to 10 scale, with 0 being “never true” and 10 being “always true.”

Patient Global Impression of Change: The Patient Global Impression of Change (PGIC) is a questionnaire that gauges patient response to medical interventions using a 7-point Likert scale ranging from “very much worse” to “very much improved,” and has been used as a secondary outcome in many pain clinical trials (Dworkin et al., Pain 2005; 113(1-2):9-19 and Dworkin et al., Pain 2008; 9(2):105-21).

Additional endpoints of the study include subject-reported outcomes and observer-reported outcomes. Subject-reported outcomes are determined from responses to a variety of questionnaires, surveys, and other forms. Various aspects of pain, FM symptoms, fatigue, anxiety, depression, catastrophizing, affect, and cognitive function are assessed. Several dosing-specific patient-reported outcomes are evaluated, including mystical experiences, psychological insight, and challenging experiences.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-64. (canceled)

65. A method of treating fibromyalgia (FM), the method comprising:
administering to a subject suffering from one or more symptoms of FM, one or more doses of psilocybin or an active metabolite thereof effective to induce a dissociative state;
providing one or more integration sessions to the subject after emergence from the dissociative state; and
assessing in the subject one or more indicators associated with the one or more symptoms of FM, dissociative state, treatment outcome, safety and/or is related to the subject, prior to and/or after administration of the one or more doses of psilocybin or an active metabolite thereof.

66. The method of claim 65, wherein the administering is by oral administration or intravenous administration.

67. The method of claim 65, wherein the one or more indicators are assessed prior to the administration as a baseline measurement, and after the administration as an outcome measurement, and the changes between the baseline measurement and the outcome measurement are determined.

68. The method of claim 67, wherein the subject exhibits amelioration of the one or more indicators between the baseline measurement and the outcome measurement.

69. The method of claim 65, wherein the one or more indicators comprises one or more of: a fibromyalgia survey score, a pain interference assessment, a sleep assessment, a chronic pain acceptance assessment, or a clinical indicator assessment.

70. The method of claim 65, wherein the one or more indicators comprises one or more of:
a fibromyalgia survey score, optionally a 2016 fibromyalgia survey questionnaire (FSQ) assessment;
a pain intensity numerical rating scale (PI-NRS) assessment, optionally an 11-item PI-NRS scale;
a PROMIS pain interference assessment;
a PainDETECT Questionnaire (PD-Q) assessment;
a chronic pain acceptance assessment, optionally measured by chronic pain acceptance questionnaire (CPAQ-8) assessment;
a pain interference assessment, optionally measured by 4-item PROMIS pain interference scale;
a Complex Medical Symptom Inventory (CMSI); a Brief Pain Inventory (BPI), optionally measured by the interference subscale (BPI-IS);
a Patient Health Questionnaire depression scale-8 (PHQ-8);
a Columbia Suicide Severity Rating Scale (C-SSRS);
a Clinician Global Impression—Improvement (CGI-I) scale;
an Emotional Breakthrough Inventory (EBI); a Acceptance and Action Questionnaire-II (AAQ-II);
a Patient Global Impression-Improvement (PGI-I);
a Hospital Anxiety and Depression Scale (HADS);
a Life Events Checklist (LEC), a Multidimensional Experiential Avoidance Questionnaire (MEAQ) a Brief Experiential Avoidance Questionnaire (BEAQ);
subject-reported mystical experiences assessment, optionally measured by a Mystical Experience Questionnaire (MEQ30);
a subject-reported challenging experiences assessment, optionally measured by a Challenging Experience Questionnaire (CEQ27);
a subject-reported psychological insight assessment, optionally measured by the Psychological Insight Questionnaire (PIQ);
a subject-reported qualitative written assessment;
functional magnetic resonance imaging (fMRI);
wrist actigraphy, optionally wherein wrist actigraphy is used to measure sleep onset latency;
wake after sleep onset; total sleep time;
sleep efficiency (%); or
an electrocardiogram (ECG).

71. The method of claim 65, wherein the one or more indicators comprises a Quantitative Sensory Testing (QST), an electroencephalogram (EEG), or both.

72. The method of claim 71, wherein the QST and the EEG are performed concurrently.

73. The method of claim 65, wherein the one or more indicators are measured during one or more of the integration sessions.

74. The method of claim 65, wherein the subject receiving treatment shows improvement in one or more symptoms of FM or one or more symptoms of FM are ameliorated.

75. The method of claim 65, wherein the subject receiving treatment shows improvements selected from among one or more of: a fibromyalgia survey score, a pain interference assessment, a sleep assessment, a chronic pain acceptance assessment, or a clinical indicator assessment.

76. The method of claim 65, wherein the subject receiving treatment shows improvements selected from among one or more of:
a fibromyalgia survey score, optionally a 2016 fibromyalgia survey questionnaire (FSQ) assessment;
a pain intensity numerical rating scale (PI-NRS) assessment, optionally an 11-item PI-NRS scale;
a PROMIS pain interference assessment;
a PainDETECT Questionnaire (PD-Q) assessment;
a chronic pain acceptance assessment, optionally measured by chronic pain acceptance questionnaire (CPAQ-8) assessment; or
a pain interference assessment, optionally measured by 4-item PROMIS pain interference scale.

77. The method of claim 65, wherein the one or more doses of psilocybin or an active metabolite thereof comprises a first dose of psilocybin.

78. The method of claim 77, wherein the first dose is between at or about 10 mg to at or about 50 mg of psilocybin; or at or about 15 mg of psilocybin.

79. The method of claim 65, wherein the one or more doses of psilocybin comprises two or more doses of psilocybin.

80. The method of claim 65, wherein the one or more doses of psilocybin comprises a second dose of psilocybin is:
between at or about 20 mg to at or about 50 mg of psilocybin;
at or about 25 mg of psilocybin;
between at or about 10 mg to at or about 20 mg of psilocybin; or
at or about 15 mg of psilocybin.

81. The method of claim 80, wherein the second dose is administered between at or about 12 days and at or about 16 days after the first dose, or at or about 14 days after the first dose.

82. The method of claim 65, further comprising providing one or more integration sessions to the subject after the one or more doses of psilocybin.

83. The method of claim 82, wherein the one or more integration sessions is provided at or about 3, 4, and/or 5 weeks after administering the first dose of psilocybin.

84. The method of claim 82, wherein the one or more integration sessions is provided at or about 1, 2, and/or 3 weeks after administering the second dose of psilocybin.
